(19) United States
(12) Patent Application Publication
(10) Pub. No.: US 2026/0217700 A1
(43) Pub. Date: Jul. 30, 2026

(54) KINESIN KIF18A INHIBITOR AND USE THEREOF

(71) Applicant: SHANGHAI APEIRON THERAPEUTICS COMPANY LIMITED, Shanghai (CN)

(72) Inventors: Yisong XIAO, Shanghai (CN); Xiaohui GU, Shanghai (CN); Kunmin LAI, Shanghai (CN)

(73) Assignee: SHANGHAI APEIRON THERAPEUTICS COMPANY LIMITED, Shanghai (CN)

(21) Appl. No.: 18/865,359

(22) PCT Filed: May 11, 2023
(86) PCT No.: PCT/CN2023/093541

§ 371 Date: Nov. 13, 2024

(30) Foreign Application Priority Data
May 13, 2022 (CN) 202210520744.0
Jul. 25, 2022 (CN) 202210878192.0
Sep. 27, 2022 (CN) 202211183092.2
Dec. 2, 2022 (CN) 202211537992.2
Jan. 16, 2023 (CN) 202310071265.X
Mar. 22, 2023 (CN) 202310288871.7
Apr. 27, 2023 (CN) 202310475180.8

(51) Int. Cl. C07D 471/04; A61K 31/4709; A61K 31/506; C07D 401/14; C07D 405/14; C07D 417/14

(52) U.S. Cl. CPC C07D 471/04; A61K 31/4709; A61K 31/506; C07D 401/14; C07D 405/14; C07D 417/14

(57) ABSTRACT

A KIF18 inhibitor and its synthetic method are provided. Compounds provided are capable of regulating the KIF18A protein, thereby influencing cell cycle and proliferation processes for the treatment of cancers and cancer-related diseases. And pharmaceutical compositions containing the compounds and methods for treating conditions associated with KIF18A activity are also provided.

### CROSS REFERENCE TO THE RELATED APPLICATIONS

This application is the national phase entry of International Application No. PCT/CN2023/093541, filed on May 11, 2023, which is based upon and claims priority to Chinese Patent Application No. 202210520744.0, filed on May 13, 2022, Chinese Patent Application No. 202210878192.0, filed on Jul. 25, 2022, Chinese Patent Application No. 202211183092.2, filed on Sep. 27, 2022, Chinese Patent Application No. 202211537992.2, filed on Dec. 2, 2022, Chinese Patent Application No. 202310071265.X, filed on Jan. 16, 2023, Chinese Patent Application No. 202310288871.7, filed on Mar. 22, 2023, and Chinese Patent Application No. 202310475180.8, filed on Apr. 27, 2023, the entire contents of which are incorporated herein by reference.

### TECHNICAL FIELD

The present invention belongs to the field of medicine and relates to a class of kinesin KIF18A inhibitors and their use for inhibiting cancer cell proliferation and treating cancers.

### BACKGROUND

KIF18A is a member of the kinesin-8 family. It moves towards the plus ends of microtubules within cells using microtubules as tracks, relying on the energy released from ATP hydrolysis. Upon reaching the plus ends of microtubules, KIF18A regulates the dynamic instability of microtubules and exerts an activity similar to that of microtubule depolymerase. During mitosis, KIF18A regulates spindle microtubule dynamics and chromosome amplitude, playing a critical role in the timely completion of chromosome alignment at mitosis, maintenance of genomic stability and successful completion of mitosis.

The KIF18A gene belongs to the kinesin-8 subfamily and is a plus-end directed motor. KIF18A is believed to influence the dynamics of the plus ends of centromeric microtubules, thereby regulating correct chromosome orientation and spindle tension. Depletion of human KIF18A results in longer spindles, increased chromosome oscillation at metaphase and activated mitotic spindle assembly checkpoint in HeLa cervical cancer cells (MI Mayr et al., Current Biology 17, 488-98, 2007). KIF18A appears to be a viable target for cancer therapy. KIF18A is overexpressed in multiple types of cancers, including but not limited to colon, breast, lung, pancreatic, prostatic, bladder, head, neck, cervical and ovarian cancers. Furthermore, in cancer cell lines, gene deletion or knockout or KIF18A inhibition affects the mitotic spindle apparatus. In particular, inhibition of KIF18A has been found to induce mitotic cell arrest, a vulnerability known to promote mitotic cell death via apoptosis, mitotic catastrophe or death following polyphasic-driven lethality or interphase mitotic slippage. As a result, there is a strong interest in finding inhibitors of KIF18A protein. Therefore, inhibition of KIF18A ATPase activity is a promising approach to developing new anticancer agents.

### SUMMARY

The present invention belongs to the field of medicine and relates to a class of kinesin KIF18A inhibitors, specifically to the said compounds or their stereoisomers, tautomers, mesomers, racemates, enantiomers, diastereoisomers or their mixture forms or pharmaceutically acceptable salts, co-crystals, metabolites, solvates, prodrugs or isotopic labels, their preparation methods and pharmaceutical compositions containing such compounds and their use as therapeutic agents, especially the use to inhibit cancer cell proliferation and treat cancers.

The present invention provides a novel class of compounds that regulate KIF18A protein, alone or in microtubule-bound complexes, for the treatment of KIF18A-mediated disorders and/or diseases, including cancer, inflammation or ciliopathy.

The compounds of the present invention exhibit MT-based regulatory activity on KIF18A, specifically inhibitory activity on KIF18A. To this end, the present invention also provides the use of these compounds and their pharmaceutically acceptable salts in the preparation and manufacture of pharmaceutical compositions or medicines for therapeutic, prophylactic, acute or chronic treatment of KIF18A-mediated diseases and disorders (including but not limited to cancers).

The present invention provides an Example: a compound having the structure of formula (I), or its pharmaceutically acceptable salts, stereoisomers, isotope isomers, prodrugs, hydrates, or solvates:

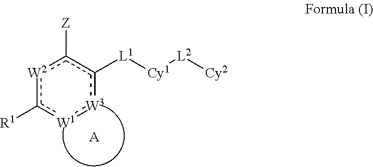

wherein, W¹ represents CR^{W1} or N;
wherein, W² represents CR^{W2} or N;
wherein, W³ represents CR^{W3} or N;
wherein, Z represents —CR^{T}R^{T′} or —NR^{S}R^{S′};
wherein, R^{T} and R^{T′}, together with the C atom they are attached to, form a ring having a structure selected from the following:

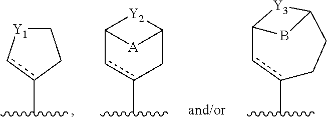

wherein, R^{S} and R^{S′}, together with the N atom they are attached to, form a ring having a structure selected from the following:

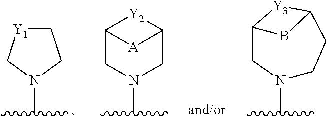

wherein, Y¹, Y² and Y³ each independently represent —(CR^{a}R^{b})ₒ—(NR^{a})ₚ—(CR^{a′}R^{b′})_{q}—; wherein, A and B each independently represent —(CR^{a}R^{b})ₘ—;

Wherein L^{f} represents —C(O)NH—, —HNC(O)—, 5-6 membered heteroaryl,

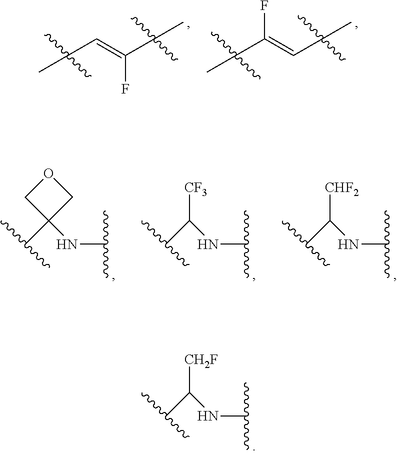

wherein, L² represents absence, —C₁-C₆ alkylene-, —NR^{a}—, —NR^{a}(C₁-C₆ alkylene)-, —C(O)NR^{a}(C₁-C₆ alkylene)-, —O—, —O—(C₁-C₆ alkylene), —S—, —S(O)—, —S(O)₂—, —S(O)₂NR^{a}— or —S(O)(NR^{a})—;
wherein, R¹ represents L³-R³;
wherein, L³ represents absence, C₁-C₆ alkylene, —NR^{a}—, —NR^{a}SO₂—, —SO₂NR^{a}_, —S(═O)(NR^{a})—, —P(O)(OR^{a})₂, —NR^{a}P(O)(OR^{a})₂ or —NR^{a}P(O)(R^{a})₂;
wherein, R³ represents absence, hydrogen, or C₁-C₆ alkyl or C₃-C₆ cycloalkyl, wherein the C₁-C₆ alkyl or C₃-C₆ cycloalkyl can be optionally independently substituted with 0-3 substituents selected from halogen, —OR^{a}, —NRaRb, cyano and —O—C₁-C₆ haloalkyl;
wherein, R^{W1}, R^{W2} and R^{W3} each independently represent hydrogen, halogen, cyano, nitro, hydroxy(C₁-C₆ alkyl), C₁-C₆ alkyl, C₃-C₈ cycloalkyl, C₁-C₆ haloalkyl, —OR^{a}, —SO₃R^{a}, —S(O)R^{a}, —O—C₁-C₆ haloalkyl, —SR, —SF₅ or —NR^{a}R^{b};
wherein, Cy^{f} represents 6-12-membered aryl or 5-12-membered heteroaryl; the Cy^{f} may be optionally substituted with 0-3 substituents selected from the following: halogen, C₁-C₆ alkyl, C₁-C₆ haloalkyl, hydroxy(C₁-C₆ alkyl), OR^{a}, —O—(C₁-C₆ haloalkyl), 5-6-membered heteroaryl, phenyl, —SR^{a}, —SF₅, cyano, nitro, —NR^{a}R^{b}, —NR^{a}C(O)R^{b}, —C(O)NR^{a}R^{b}, —OC(O)R^{a}, —C(O)R^{a}, —P(O)R^{a}R^{b}, —C(O)OR^{a}, —S(O)R^{a}, —S(O)₂R^{a} and —S(O)₂NR^{a}R^{b};
wherein, Cy² represents a 3-12-membered saturated or unsaturated monocyclic or bicyclic ring which may optionally contain 0-3 heteroatoms selected from O, N and S; the Cy² may be optionally substituted with 0-3 substituents selected from the following: halogen, C₁-C₆ haloalkyl, C₁-C₆ haloalkyl, hydroxy (C₁-C₆ alkyl), —OR^{a}, —O—(C₁-C₆ haloalkyl), —SR^{a}, —SF₅, cyano, nitro, —NR^{a}R^{b}, —NR^{a}C(O)R, —C(O)NR^{a}R, —OC(O)R^{a}, —C(O)OR^{a}, —S(O)R^{a}, —S(O)₂R^{a} and —S(O)₂NR^{a}R^{b};
wherein, ring A represents a saturated or unsaturated 4-12-membered ring, preferably a 5-12-membered ring; the ring may optionally contain 0-3 heteroatoms selected from O, N and S; the ring A may be optionally substituted with 0-3 substituents selected from the following: halogen, C₁-C₆ alkyl, C₁-C₆ haloalkyl, hydroxy C₁-C₆ alkyl, —OR^{a}, —O—C₁-C₆ haloalkyl, —SR^{a}, —SF₅, cyano, nitro, —NR^{a}R^{b}, —NR^{a}C(O)R^{b}, —C(O)NR^{a}R, —OC(O)R^{a}, —C(O)OR^{a}, —S(O)R^{a}, —S(O)₂R^{a} and —S(O)₂NR^{a}R^{b};
wherein, R^{a}, R^{b}, R^{a′} and R^{b′} each independently represent hydrogen, halogen, C₁-C₆ alkyl, C₃-C₆ cycloalkyl, C₁-C₆ haloalkyl or hydroxy (C₁-C₆ alkyl); Or R^{a} and R^{b}, together with the atom they are attached to, form a 3-6-membered saturated or unsaturated ring which may optionally contain 0-2 heteroatoms selected from O, S and N; OR R^{α′} and R^{b′}, together with the atom they are attached to, form a 3-6-membered saturated or unsaturated ring which may optionally contain 0-2 heteroatoms selected from O, S and N; wherein, m, o, p and q independently represent integers from 0 to 3.

In addition, the present invention provides a compound having the structure of formula (II) or its pharmaceutically acceptable salts, stereoisomers, isotope isomers, prodrugs, hydrates or solvates:

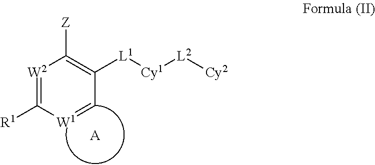

wherein, W² represents CR^{W1} or N;
wherein, W² represents CR^{W2} or N;
wherein, Z represents —CR^{T}R^{T′} or —NR^{S}R^{S′};
wherein, R^{T} and R^{T′}, together with the C atom they are attached to, form a ring having a structure selected from the following:

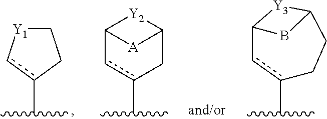

wherein, R^{S} and R^{S′}, together with the N atom they are attached to, form a ring having a structure selected from the following:

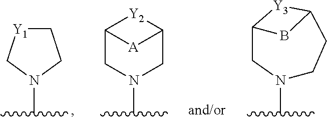

wherein, Y¹, Y² and Y³ each independently represent —(CR^{a}R^{b})ₒ—(NR^{a})ₚ—(CR^{a′}R^{b′})_{q}—; wherein, A and B each independently represent —(CR^{a}R^{b})ₘ—;

Wherein L¹ represents —C(O)NH—, —HNC(O)—, 5-6 membered heteroaryl,

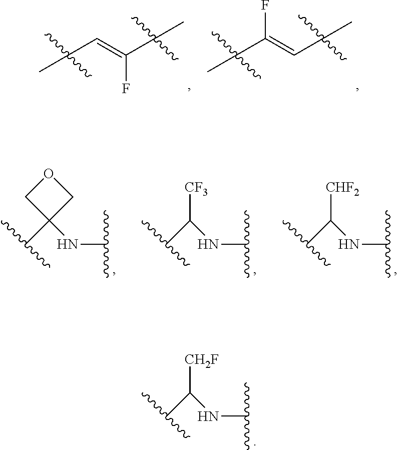

wherein, L² represents absence, —C₁-C₆ alkylene-, —NR^{a}—, —NR^{a}(C₁-C₆ alkylene)-, —C(O)NR^{a}(C₁-C₆ alkylene)-, —O—, —O—(C₁-C₆ alkylene), —S—, —S(O)—, —S(O)₂—, —S(O)₂NR^{a}— or —S(O)(NR^{a})_;
wherein, R¹ represents L³-R³;
wherein, L³ represents absence, C₁-C₆ alkylene, —NR^{a}—, —NR^{a}SO₂—, —SO₂NR^{a}—, —S(═O)(NR^{a})—, —P(O)(OR^{a})₂, —NR^{a}P(O)(OR^{a})₂ or —NR^{a}P(O)(R^{a})₂;
wherein, R³ represents absence, hydrogen, or C₁-C₆ alkyl or C₃-C₆ cycloalkyl, wherein the C₁-C₆ alkyl or C₃-C₆ cycloalkyl can be optionally independently substituted with 0-3 substituents selected from halogen, —OR^{a}, —NR^{a}R^{b}, cyano and —O—C₁-C₆ haloalkyl;
wherein, R^{W1} and R^{W2} each independently represent hydrogen, halogen, cyano, nitro, hydroxy C₁-C₆ alkyl, C₁-C₆ alkyl, C₃-C₅ cycloalkyl, C₁-C₆ haloalkyl, —OR, —SO₃R^{a}, —S(O)R^{a}, —O—C₁-C₆ haloalkyl, —SR^{a}, —SF₅ or —NR^{a}R^{b};
wherein, Cy¹ represents 6-12-membered aryl or 5-12-membered heteroaryl; the Cy¹ may be optionally substituted with 0-3 substituents selected from the following: halogen, C₁-C₆ alkyl, C₁-C₆ haloalkyl, hydroxy(C₁-C₆ alkyl), OR^{a}, —O—(C₁-C₆ haloalkyl), 5-6-membered heteroaryl, phenyl, —SR^{a}, —SF₅, cyano, nitro, —NR^{a}R^{b}, —NR^{a}C(O)R^{b}, —C(O)NR^{a}R^{b}, —OC(O)R^{a}, —C(O)R^{a}, —P(O)R^{a}R^{b}, —C(O)OR^{a}, —S(O)R^{a}, —S(O)₂R^{a} and —S(O)₂NR^{a}R^{b};
wherein, Cy² represents a 3-12-membered saturated or unsaturated monocyclic or bicyclic ring which may optionally contain 0-3 heteroatoms selected from O, N and S; the Cy² may be optionally substituted with 0-3 substituents selected from the following: halogen, C₁-C₆ haloalkyl, C₁-C₆ haloalkyl, hydroxy (C₁-C₆ alkyl), —OR^{a}, —O—(C₁-C₆ haloalkyl), —SR^{a}, —SF₅, cyano, nitro, —NR^{a}R^{b}, —NR^{a}C(O)R, —C(O)NR^{a}R^{b}, —OC(O)R^{a}, —C(O)OR^{a}, —S(O)R^{a}, —S(O)₂R^{a} and —S(O)₂NR^{a}R^{b};
wherein, ring A represents a saturated or unsaturated 4-12-membered ring, preferably a 5-12-membered ring; the ring may optionally contain 0-3 heteroatoms selected from O, N and S; the ring A may be optionally substituted with 0-3 substituents selected from the following: halogen, C₁-C₆ alkyl, C₁-C₆ haloalkyl, hydroxy C₁-C₆ alkyl, —OR^{a}, —O—C₁-C₆ haloalkyl, —SR^{a}, —SF₅, cyano, nitro, —NR^{a}R^{b}, —NR^{a}C(O)R^{b}, —C(O)NR^{a}R, —OC(O)R^{a}, —C(O)OR^{a}, —S(O)R^{a}, —S(O)₂R^{a} and —S(O)₂NR^{a}R^{b};
wherein, R^{a}, R^{b}, R^{a′} and R^{b′} each independently represent hydrogen, halogen, C₁-C₆ alkyl, C₃-C₆ cycloalkyl, C₁-C₆ haloalkyl or hydroxy (C₁-C₆ alkyl); Or R^{a} and R^{b}, together with the atom they are attached to, form a 3-6-membered saturated or unsaturated ring which may optionally contain 0-2 heteroatoms selected from O, S and N; OR R^{a′} and R^{b′}, together with the atom they are attached to, form a 3-6-membered saturated or unsaturated ring which may optionally contain 0-2 heteroatoms selected from O, S and N; wherein, m, o, p and q independently represent integers from 0 to 3.

In a preferred Example of the present invention, W¹ represents CH or N.

In a preferred Example of the present invention, W² represents CH or N.

In a preferred Example of the present invention, L³ represents —NR^{a}SO₂—, —SO₂NR^{a}— or —S(═O)(NR^{a}).

In a preferred Example of the present invention, L³ represents —NR^{a}SO₂—.

In a preferred Example of the present invention, R³ represents hydrogen, or C₁-C₆ alkyl or C₃-C₆ cycloalkyl substituted with 0-3 substituents selected from halogen, —OR^{a}, —NR^{a}R^{b} cyano, and —O—C₁-C₆ haloalkyl.

In a preferred Example of the present invention, R³ represents C₁-C₆ alkyl substituted with 0-3 substituents selected from halogen, —OR^{a}, —NR^{a}R^{b}, cyano and —O—C₁-C₆ haloalkyl.

In a preferred Example of the present invention, Z represents NR^{S}R^{S′}.

In a preferred Example of the present invention, Z represents:

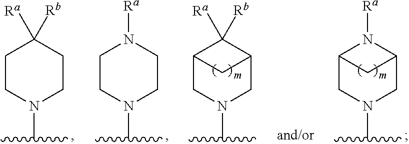

wherein, R^{a} and R^{b} each independently represent hydrogen, C₁-C₆ alkyl, C₃-C₆ cycloalkyl, C₁-C₆ haloalkyl or hydroxy (C₁-C₆ alkyl); Or R^{a} and R^{b}, together with the atom they are attached to, form a 3-6-membered saturated or unsaturated ring which may optionally contain 0-2 heteroatoms selected from O, S and N.

In a preferred Example of the present invention, Z represents:

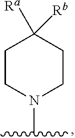

wherein, R^{a} and R^{b} each independently represent hydrogen, C₁-C₆ alkyl, C₃-C₆ cycloalkyl, C₁-C₆ haloalkyl or hydroxy (C₁-C₆ alkyl); Or R^{a} and R^{b}, together with the atom they are attached to, form a 3-6-membered saturated or unsaturated ring which may optionally contain 0-2 heteroatoms selected from O, S and N.

In a preferred Example of the present invention, Z represents.

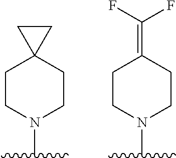 [SMILES: *N1CCC2(CC1)CC2 ; *N1CCC(=C(F)F)CC1]

In the preferred Example of the present invention, L¹ represents —C(O)NH—, —HNC(O)—, 5-6 membered heteroaryl,

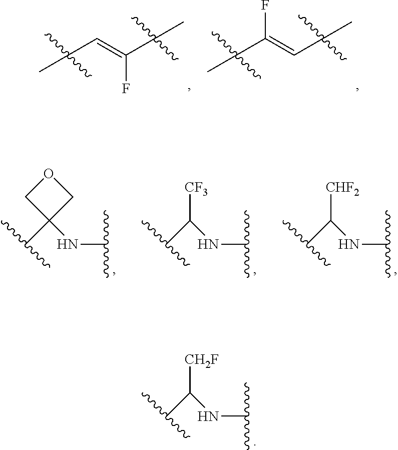

In a preferred Example of the present invention, L¹ represents —C(O)NH—.

In the preferred example of the resent invention, L¹ represents —C(O)NH—, —HNC(O)—, 5-6 membered heteroaryl,

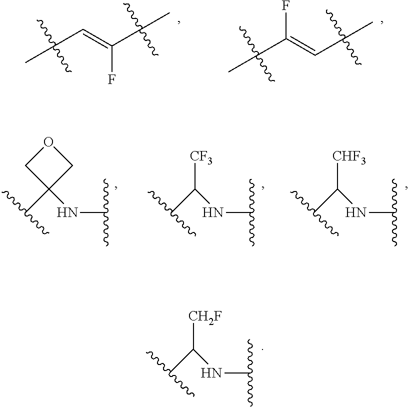

In a preferred Example of the present invention, L¹ represents any one of the following groups:

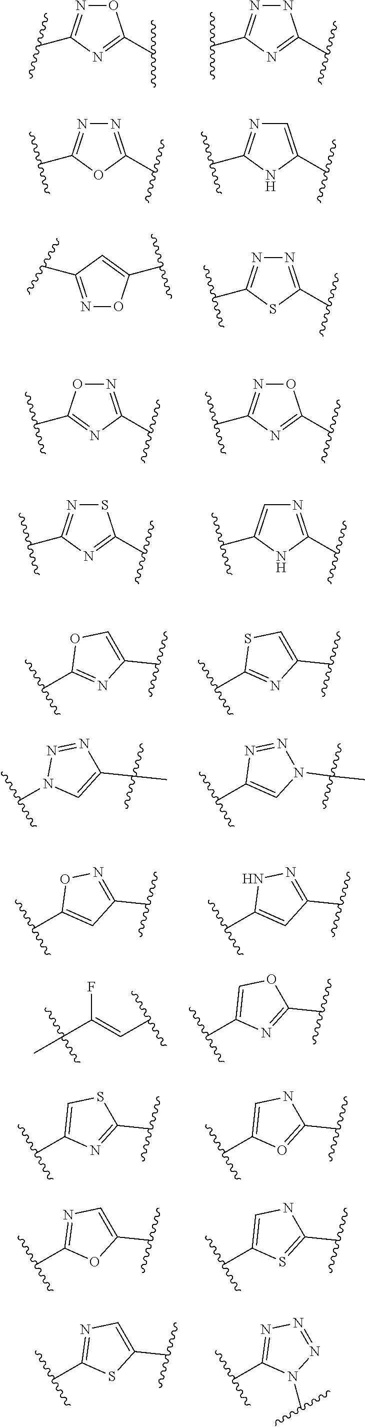

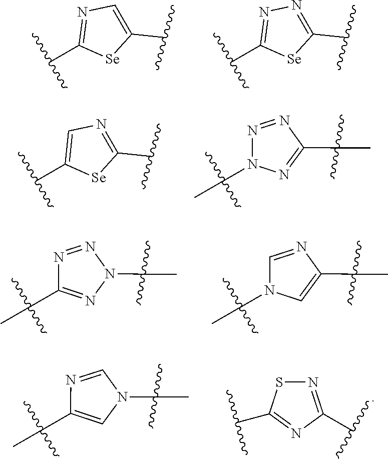

In a preferred Example of the present invention, Cy¹ represents any one of the following groups substituted with 0-3 substituents selected from halogen, C₁-C₆ alkyl, C₁-C₆ haloalkyl, hydroxy (C₁-C₆ alkyl), OR^{a}, —O—(C₁-C₆ haloalkyl), 5-6 membered heteroaryl, phenyl, —SR^{a}, —SF₅, cyano, nitro, —NR^{a}R^{b}, —NR^{a}C(O)R^{b}, —C(O)NR^{a}R^{b}, —OC(O)R^{a}, —C(O)R^{a}, —P(O)R^{a}R^{b}, —C(O)OR^{a}, —S(O)R^{a}, —S(O)₂R^{a} and —S(O)₂NR^{a}R:

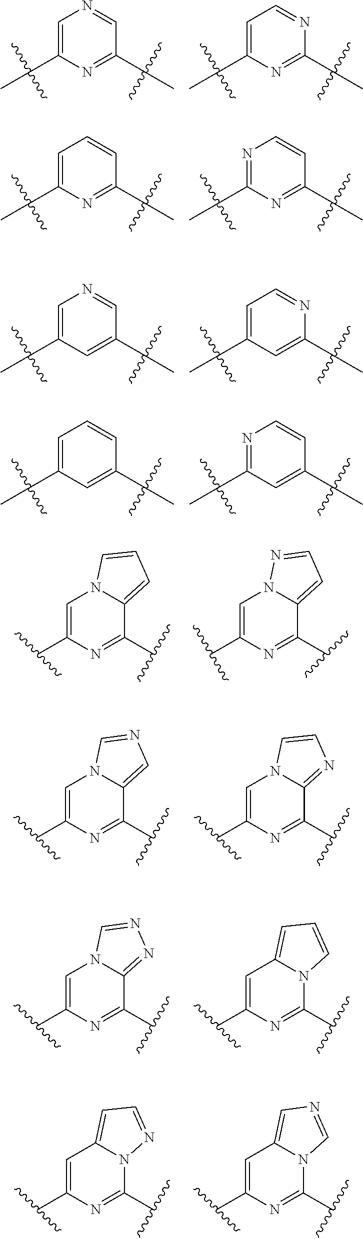

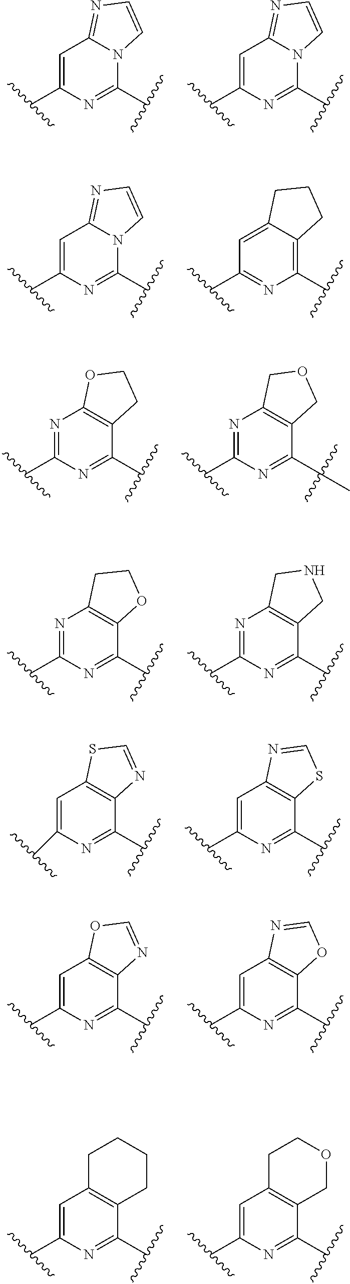

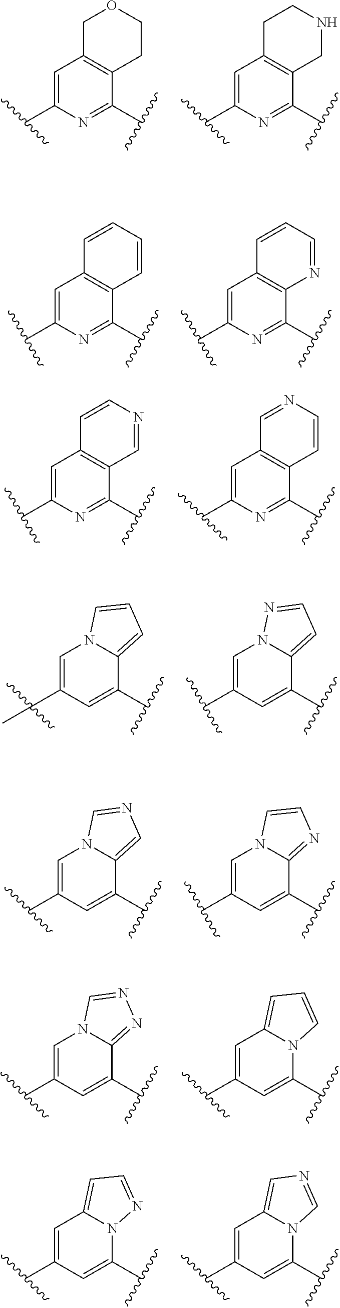

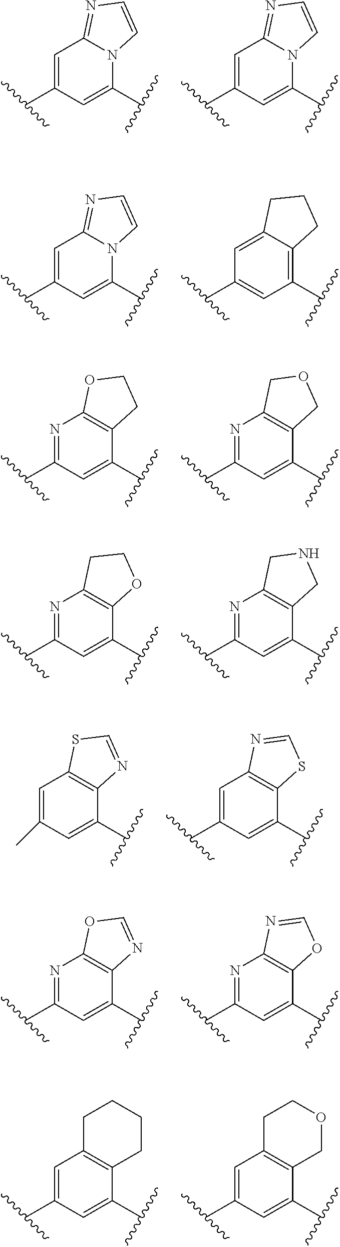

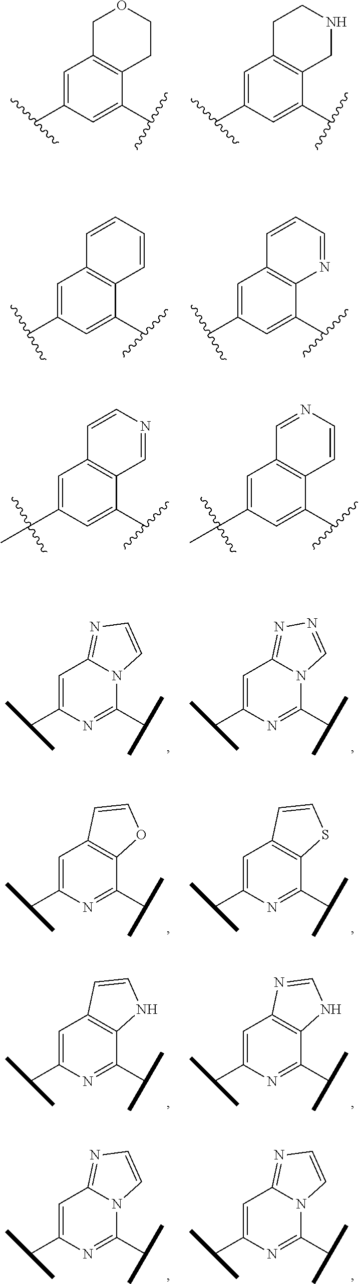

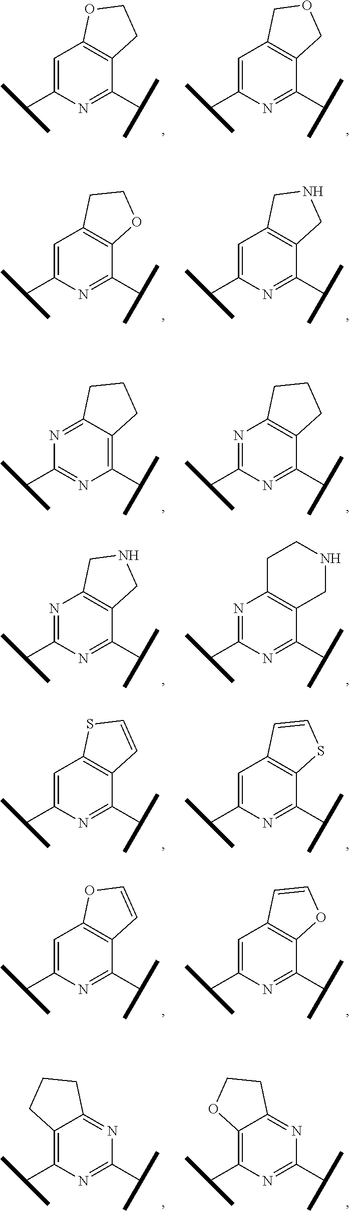

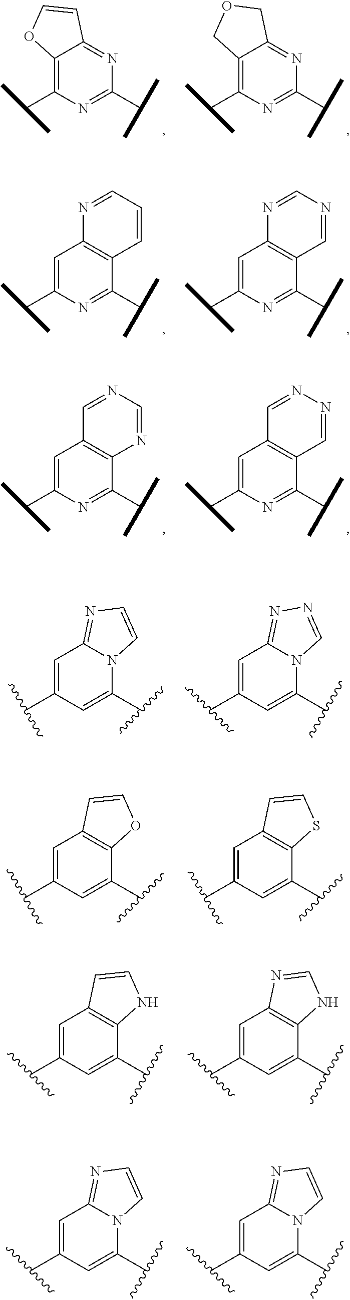

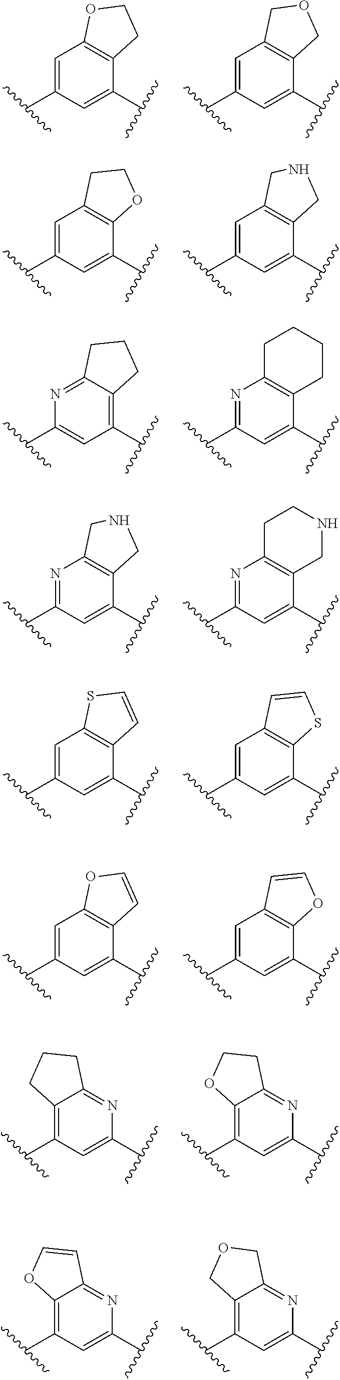

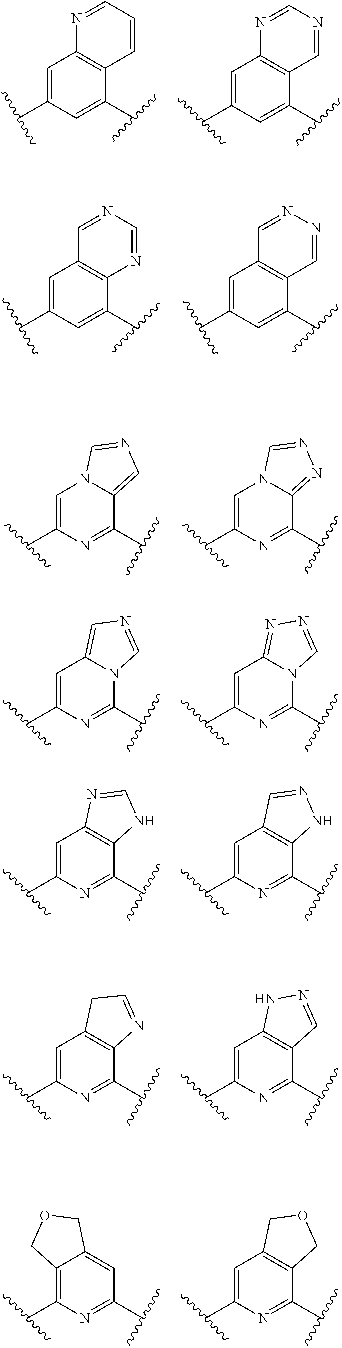

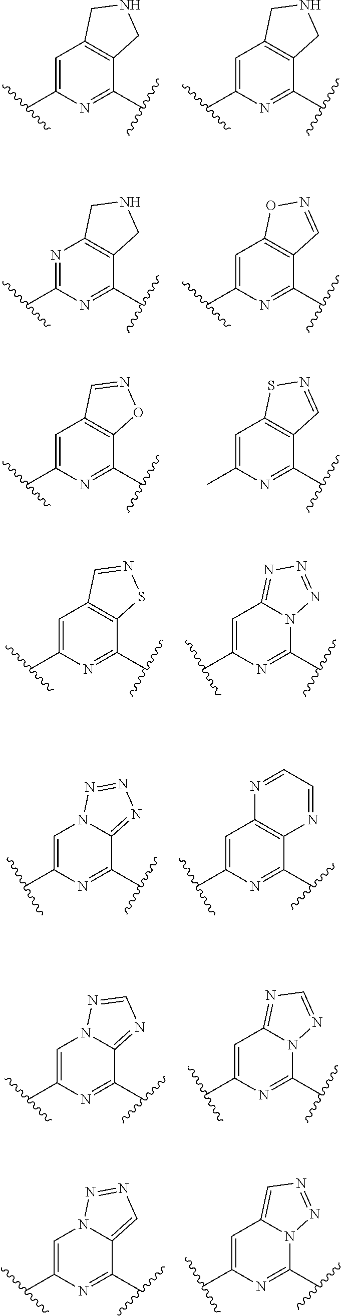

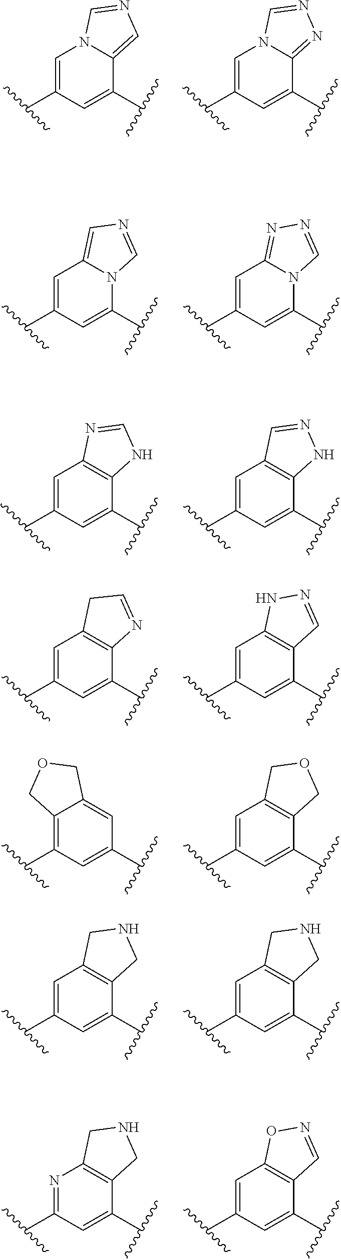

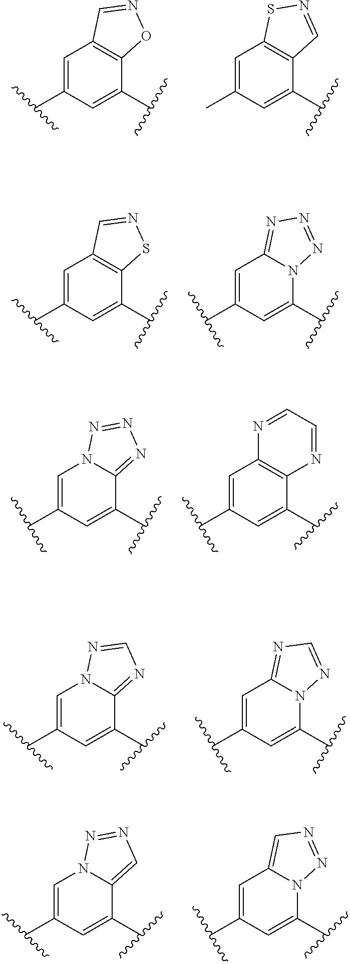

For example:

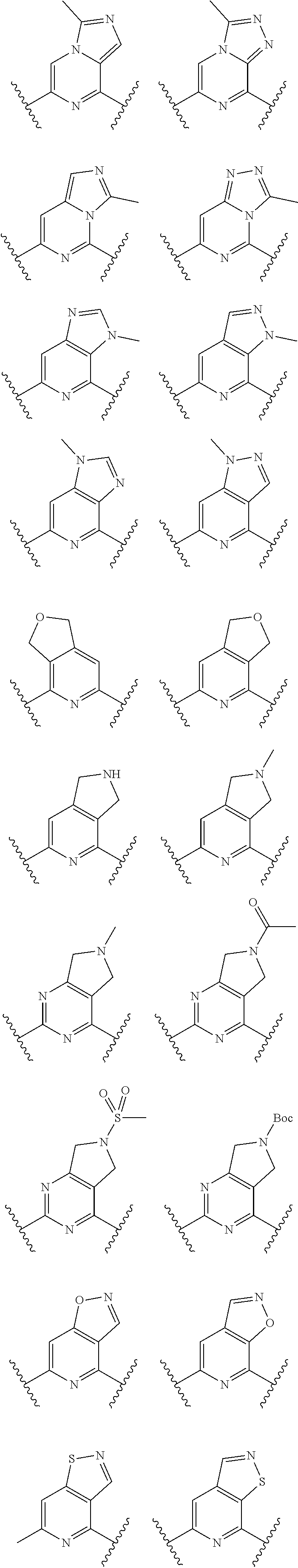

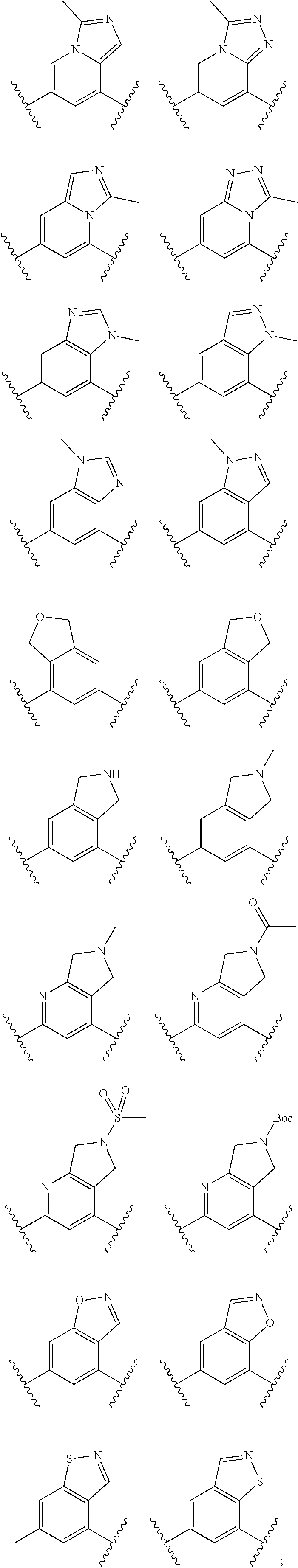

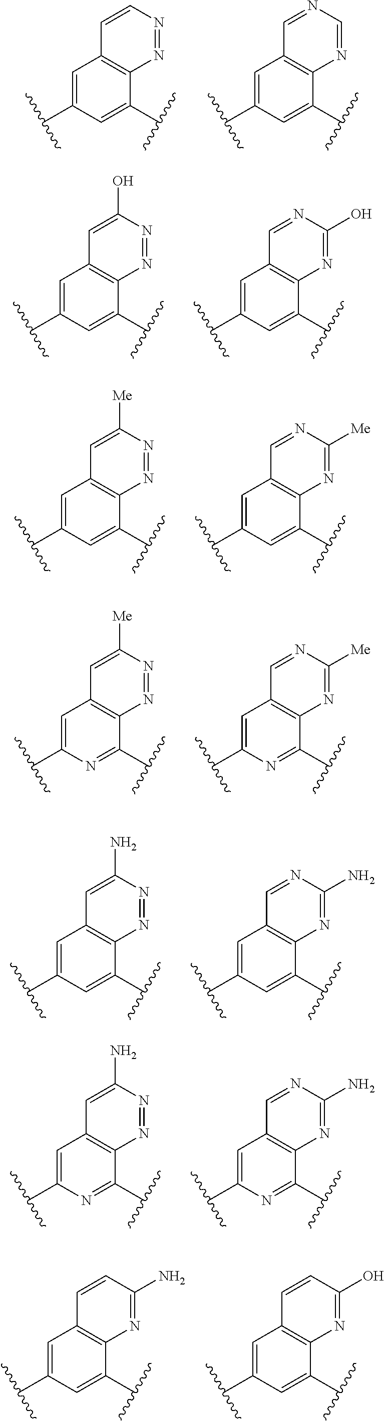

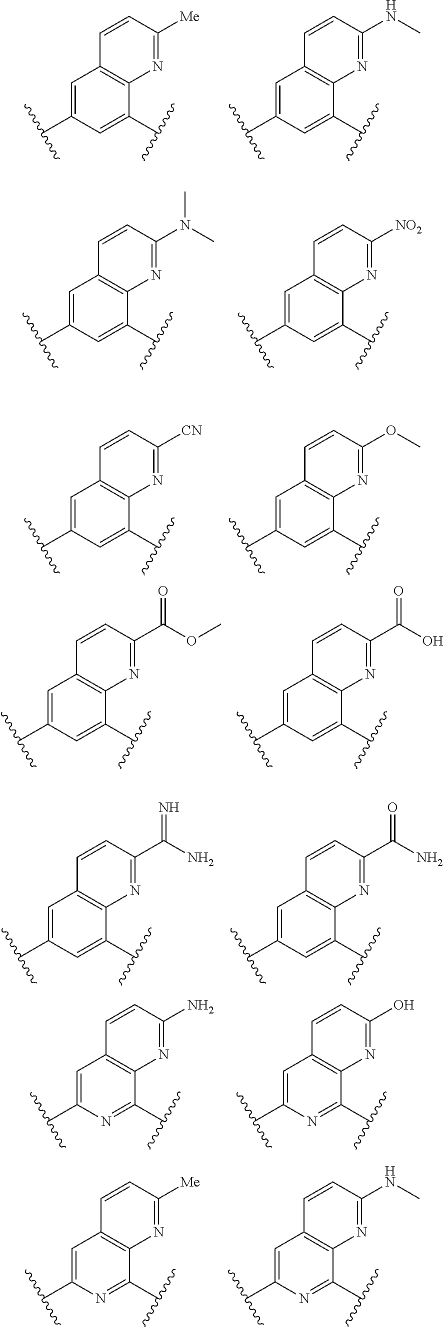

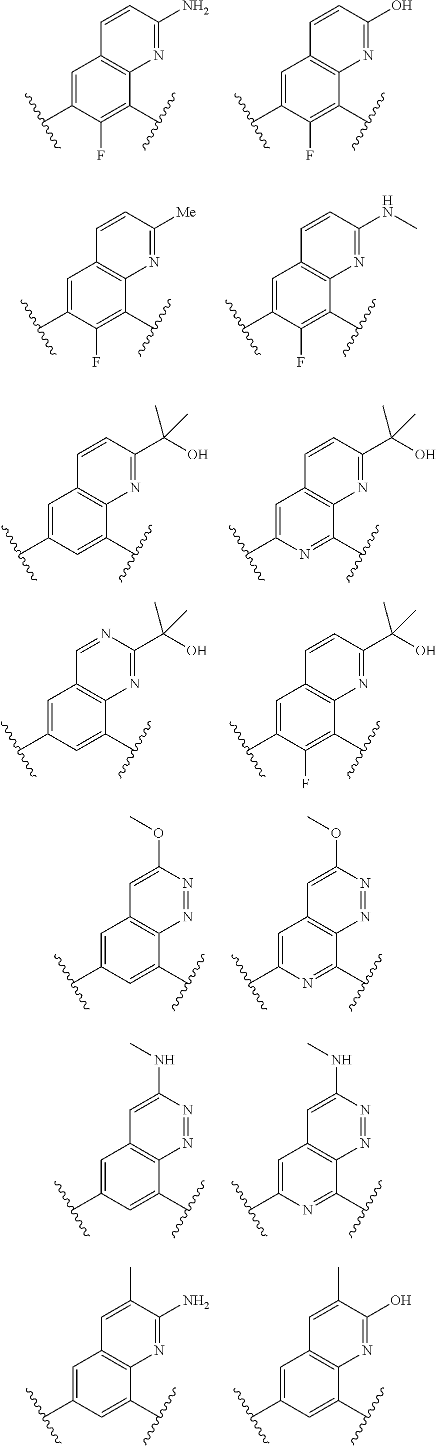

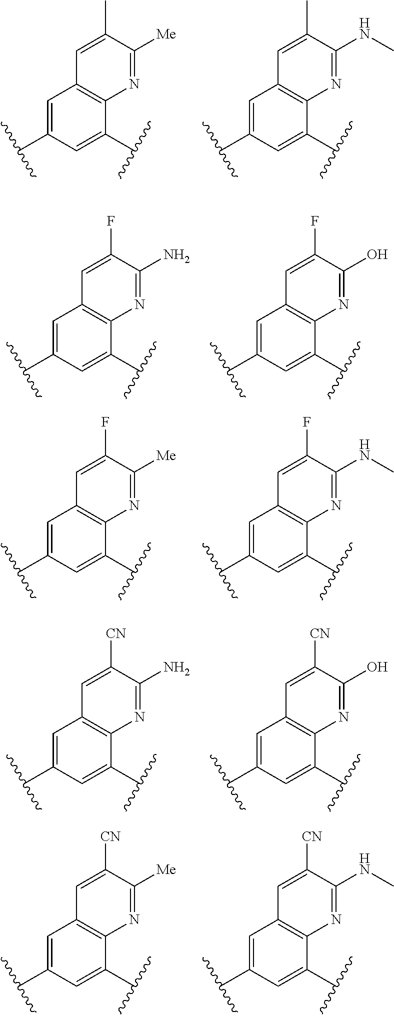

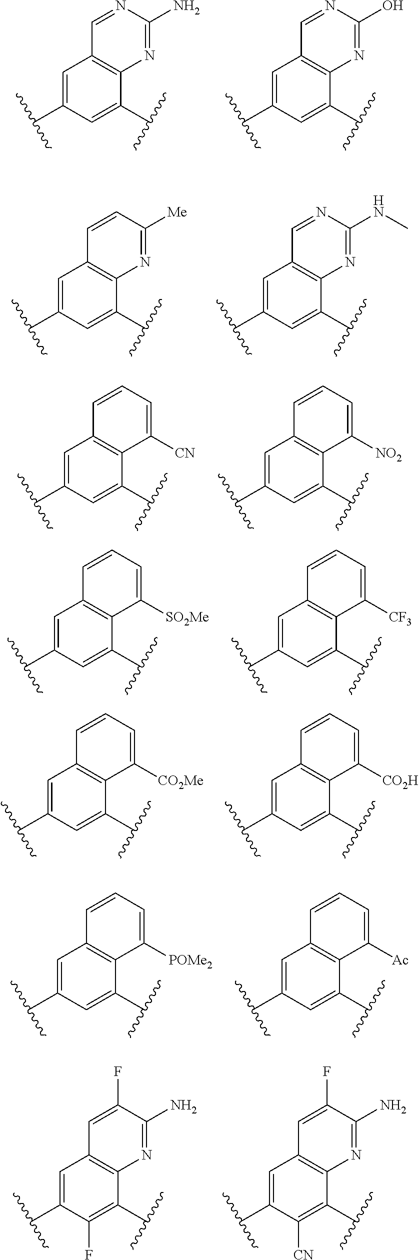

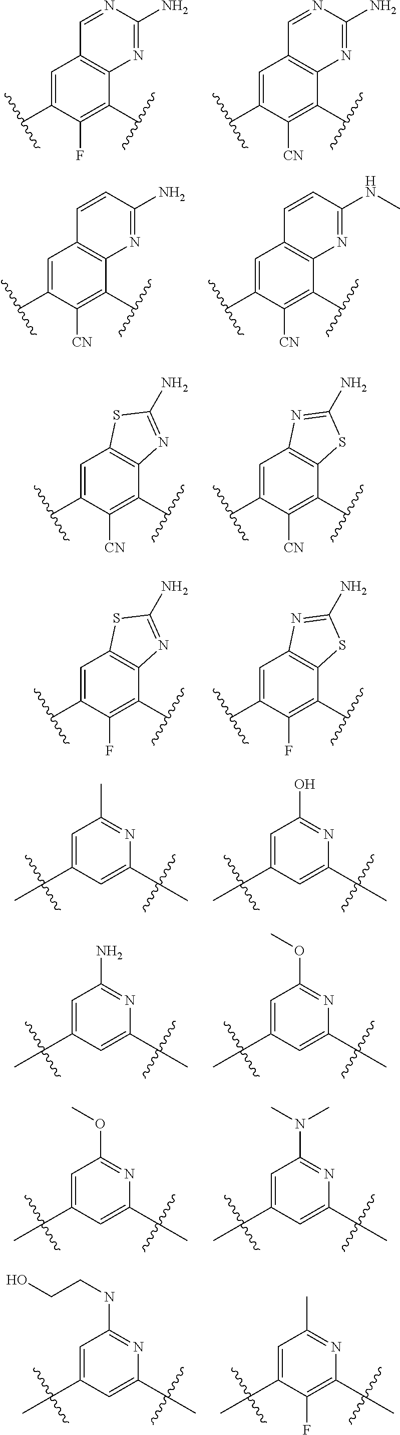

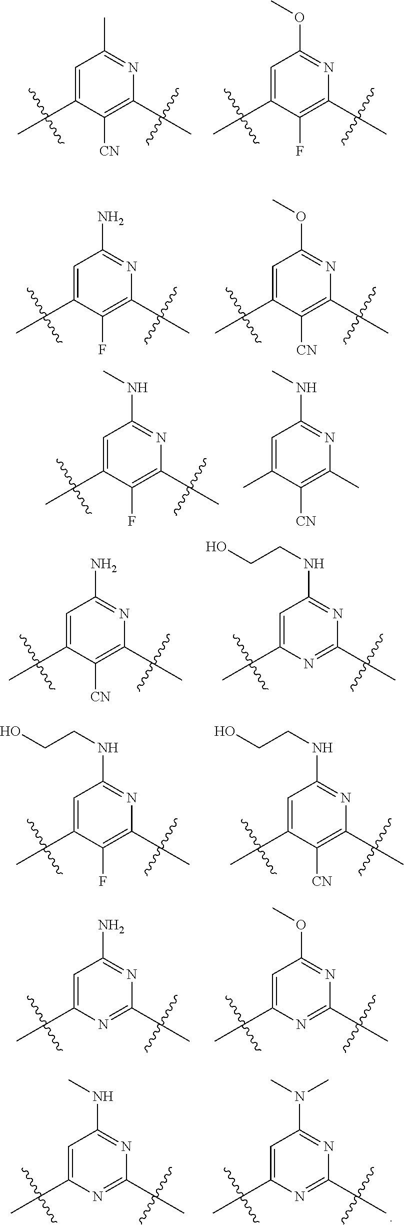

In a preferred Example of the present invention, Cy¹ represents any one of the following groups substituted with 0-3 substituents selected from halogen, C₁-C₆ alkyl, C₁-C₆ haloalkyl, hydroxy (C₁-C₆ alkyl), OR^{a}, —O—(C₁-C₆ haloalkyl), 5-6 membered heteroaryl, phenyl, —SR, —SF₅, cyano, nitro, —NR^{a}R^{b}, —NR^{a}C(O)R^{b}, —C(O)NR^{a}R, —OC(O)R^{a}, —C(O)R^{a}, —P(O)R^{a}R^{b}, —C(O)OR^{a}, —S(O)R^{a}, —S(O)₂R^{a} and —S(O)₂NR^{a}R:

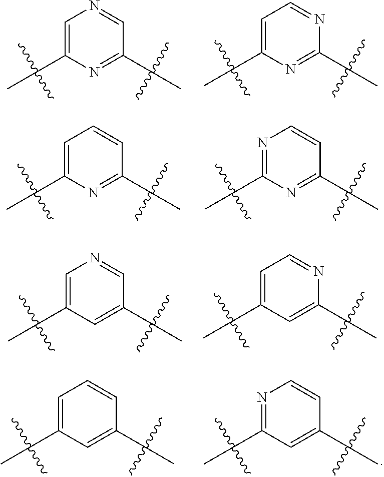

In a preferred Example of the present invention, Cy¹ represents the following group substituted with 0-3 substituents selected from halogen, C₁-C₆ alkyl, halo C₁-C₆ alkyl, hydroxy C₁-C₆ alkyl, OR^{a}, —O-halo C₁-C₆ alkyl, 5-6 membered heteroaryl, phenyl, —SR^{a}, —SF₅, cyano, nitro, —NR^{a}R^{b}, —NR^{a}C(O)R, —C(O)NR^{a}R^{b}, —OC(O)R^{a}, —C(O)R^{a}, —P(O)R^{a}R^{b}, —C(O)O R^{a}, —S(O)R^{a}, —S(O)₂R^{a} and —S(O)₂NR^{a}R^{b}:

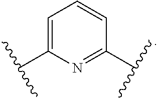 [SMILES: *c1cccc(*)n1]

In a preferred Example of the present invention, Cy¹ represents

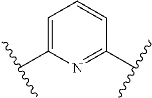 [SMILES: *c1cccc(*)n1]

optionally substituted with phenyl, pyridinyl, thiazolyl, oxazolyl, pyrazolyl, imidazolyl and N-methylpyrazolyl.

In a preferred Example of the present invention, Cy¹ represents the following groups.

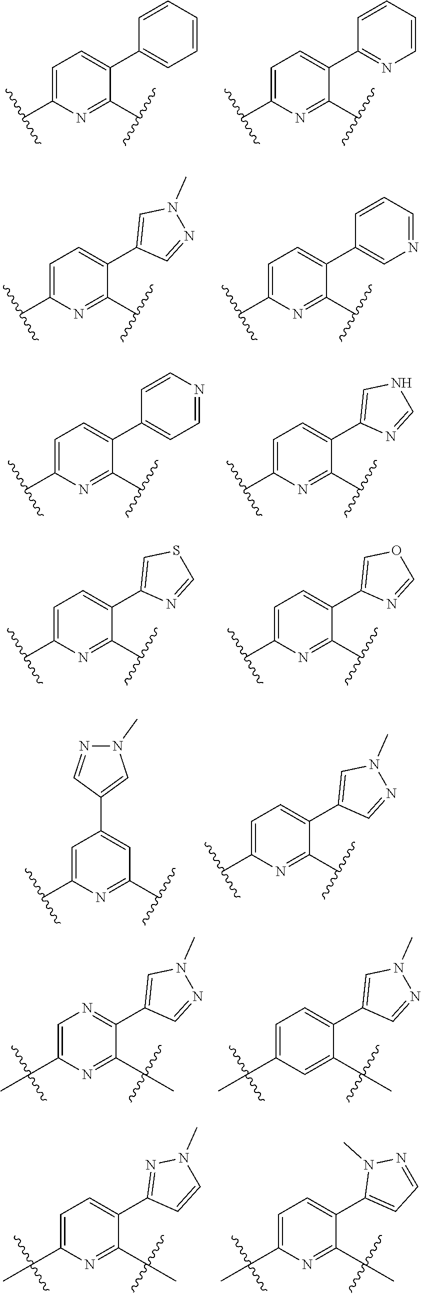

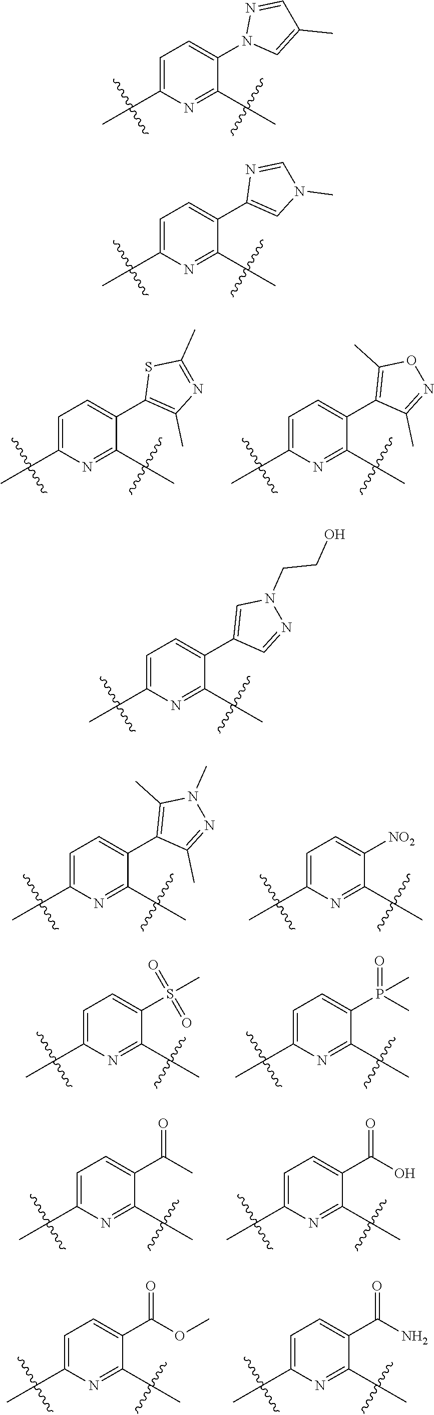

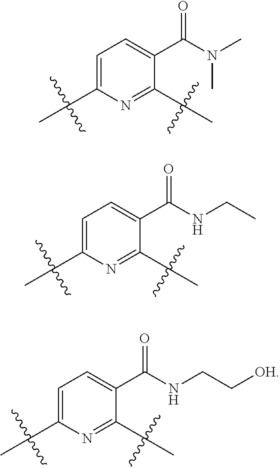 [SMILES: *c1ccc(C(=O)N(C)C)c(*)n1 ; *c1ccc(C(=O)NCC)c(*)n1 ; *c1ccc(C(=O)NCCO)c(*)n1]

In a preferred Example of the present invention, Cy¹ represents the following group substituted with 0-3 substituents selected from halogen, C₁-C₆ alkyl, halo C₁-C₆ alkyl, hydroxy C₁-C₆ alkyl, OR^{a}, —O-halo C₁-C₆ alkyl, 5-6 membered heteroaryl, phenyl, —SR^{a}, —SF₅, cyano, nitro, —NR^{a}R^{b}, —NR^{a}C(O)R^{b}, —C(O)NR^{a}R^{b}, —OC(O)R^{a}, —C(O)R^{a}, —P(O)R^{a}R^{b}, —C(O)OR^{a}, —S(O)R^{a}, —S(O)R^{a}, and —S(O)₂R^{a}R^{b}.

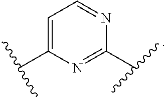 [SMILES: *c1ccnc(*)n1]

In a preferred Example of the present invention, L² represents absence, —C₁-C₆ alkylene- or —NH—.

In a preferred Example of the present invention, L² represents absence.

In a preferred Example of the present invention, Cy² may be substituted with 0-3 substituents selected from the following: halogen, cyano, nitro, hydroxy C₁-C₆ alkyl, C₁-C₆ alkyl, C₃-C₈ cycloalkyl, C₁-C₆ haloalkyl, —OR^{a}, —SO₃R^{a}, —S(O)R^{a}, —O—C₁-C₆ haloalkyl, —SR^{a}, —SF₅ or NR^{a}R^{b}-substituted morpholinyl, piperidinyl, azetidine, pyrrolidinyl, cyclopropyl, cyclobutyl, cyclopentyl, cyclohexyl, piperazinyl or tetrahydrofuryl; wherein, R^{a} and R^{b} each independently represent hydrogen, halogen, C₁-C₆ alkyl, C₃-C₈ cycloalkyl, C₁-C₆ haloalkyl or hydroxy C₁-C₆ alkyl; OR R^{1a} and R^{1b}, together with the atom they are attached to, form a 3-6 membered ring which contains 0-2 heteroatoms selected from O, N and S.

In a preferred Example of the present invention, Cy² represents

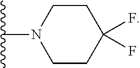 [SMILES: *N1CCC(F)(F)CC1]

In a preferred Example of the present invention, ring A represents benzene ring or

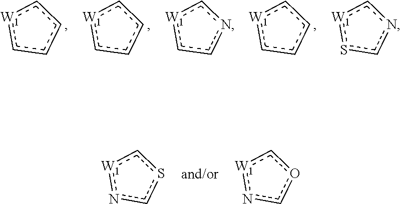

wherein, W¹ represents CH or N,
wherein, the dashed line represents a single or double bond;

The ring A may be optionally substituted with 0-3 substituents selected from the following: halogen, C₁-C₆ alkyl, C₁-C₆ haloalkyl, hydroxy C₁-C₆ alkyl, —OR^{a}, —O—C₁-C₆ haloalkyl, —SR, —SF₅, cyano, nitro, —NR^{a}R^{b}, —NR^{a}C(O)R, —C(O)NR^{a}R, —OC(O)R^{a}, —C(O)OR^{a}, —S(O)R^{a}, —S(O)₂R^{a} and —S(O)₂NR^{a}R^{b}.

In a preferred Example of the present invention, ring A represents

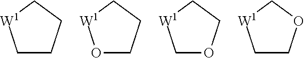

wherein, W¹ represents CH or N;

The ring A may be optionally substituted with 0-3 substituents selected from the following: halogen, C₁-C₆ alkyl, C₁-C₆ haloalkyl, hydroxy C₁-C₆ alkyl, —OR^{a}, —O—C₁-C₆ haloalkyl, —SR^{a}, —SF₅, cyano, nitro, —NR^{a}R^{b}, —NR^{a}C(O)R^{b}, —C(O)NR^{a}R^{b}, —OC(O)R^{a}, —C(O)OR^{a}, —S(O)R^{a}, —S(O)₂R^{a} and —S(O)₂NR^{a}R^{b}.

In a preferred Example of the present invention, ring A represents a benzene ring, pyridine ring, pyrazine ring, cyclobutane ring, cyclopentane ring, tetrahydrofuran ring, oxazole ring, thiazole ring, thiophene ring, pyrazole ring, imidazole ring, piperidine ring, hexahydropyran ring and cyclohexane ring;

The ring A may be optionally substituted with 0-3 substituents selected from the following: halogen, C₁-C₆ alkyl, C₁-C₆ haloalkyl, hydroxy C₁-C₆ alkyl, —OR^{a}, —O—C₁-C₆ haloalkyl, —SR^{a}, —SF₅, cyano, nitro, —NR^{a}R^{b}, —NR^{a}C(O)R, —C(O)NR^{a}R, —OC(O)R^{a}, —C(O)OR^{a}, —S(O)R^{a}, —S(O)₂R^{a} and —S(O)₂NR^{a}R^{b}.

In a preferred Example of the present invention, ring A, together with its fused

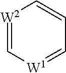

ring, forms the following structures:

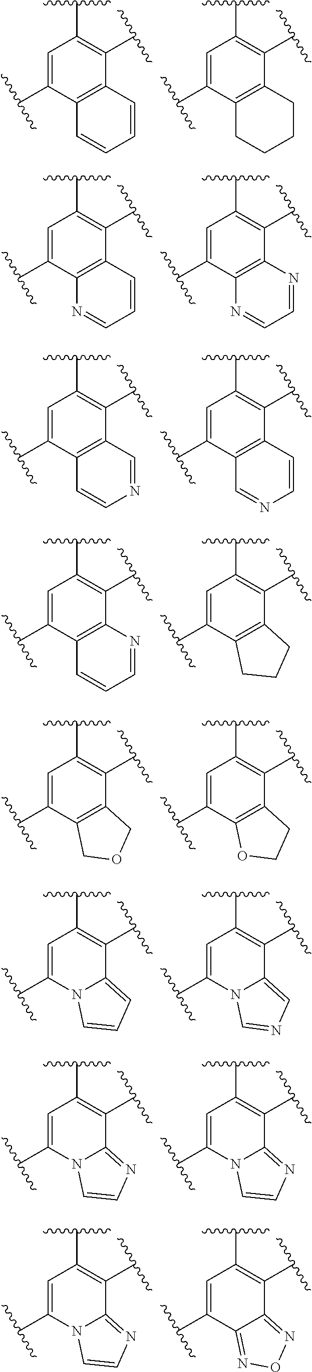

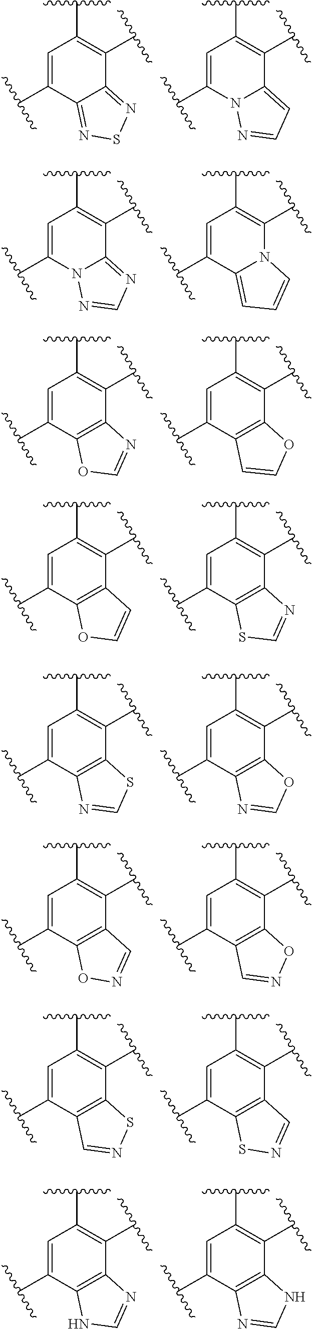

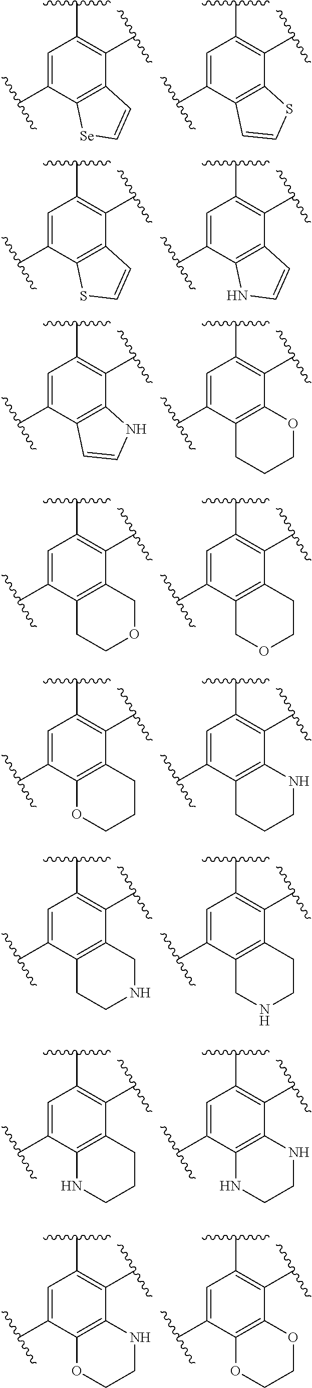

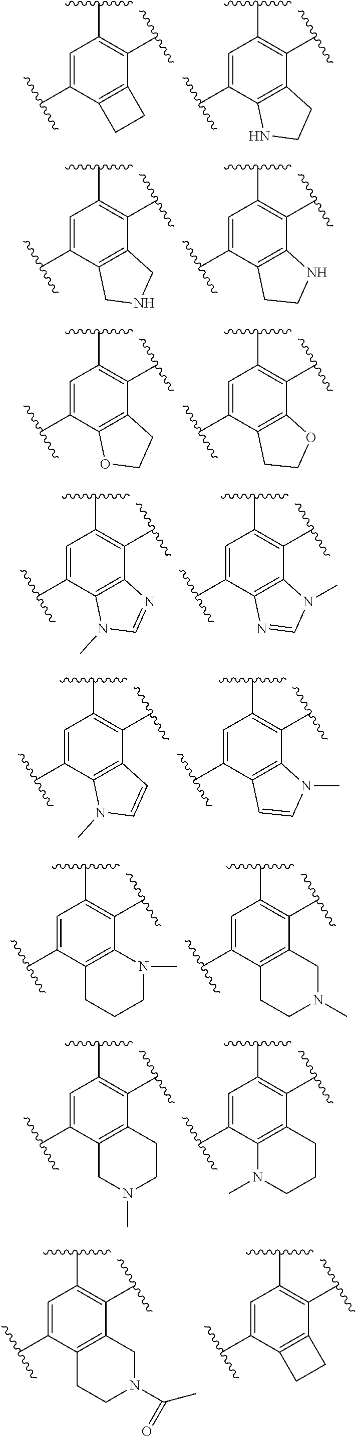

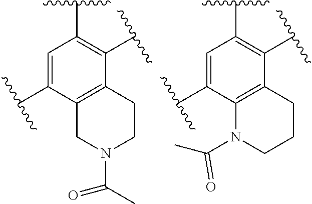

The ring A may be optionally substituted with 0-3 substituents selected from the following: halogen, C₁-C₆ alkyl, C₁-C₆ haloalkyl, hydroxy C₁-C₆ alkyl, —OR^{a}, —O—C₁-C₆ haloalkyl, —SR, —SF₅, cyano, nitro, —NR^{a}R^{b}, —NR^{a}C(O)R^{b}, —C(O)NR^{a}R^{b}, —OC(O)R^{a}, —C(O)OR^{a}, —S(O)R^{a}, —S(O)₂R^{a} and —S(O)₂NR^{a}R^{b}.

In a preferred Example of the present invention, ring A, together with its fused

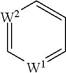

ring forms the following structures:

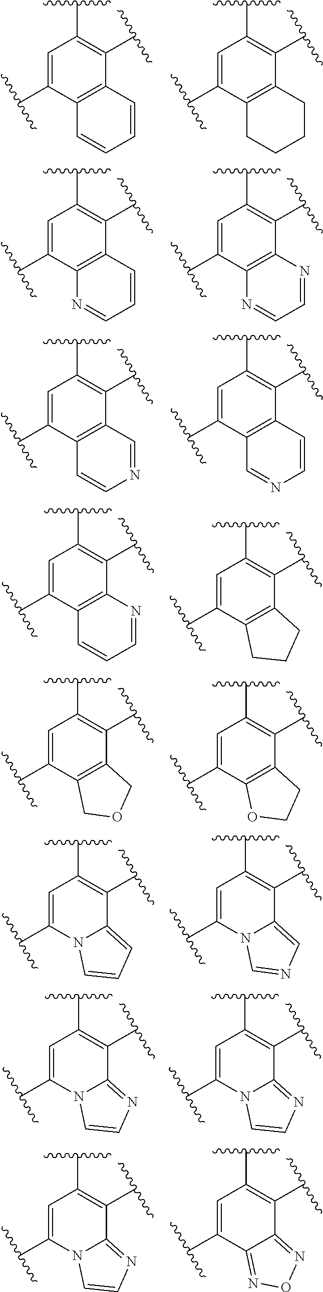

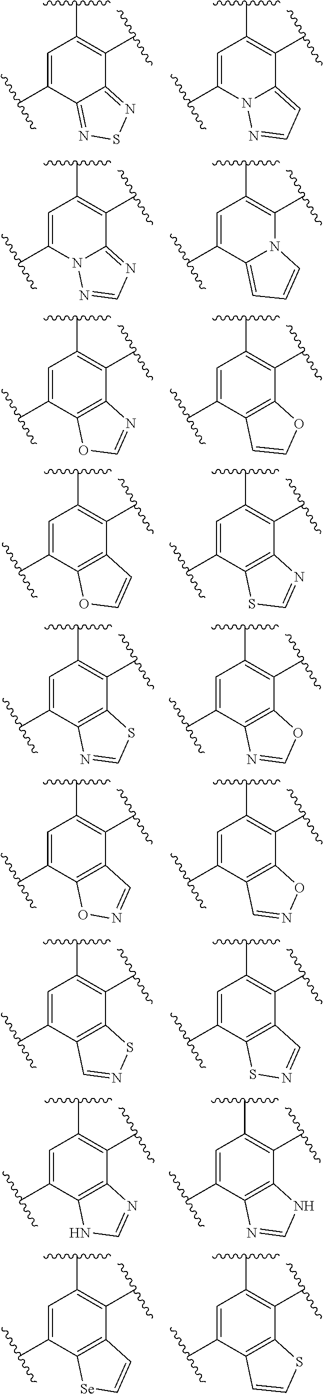

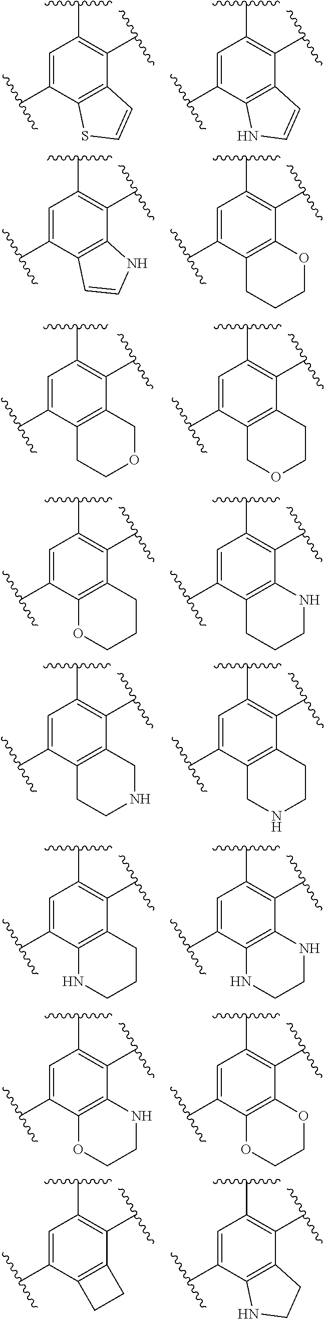

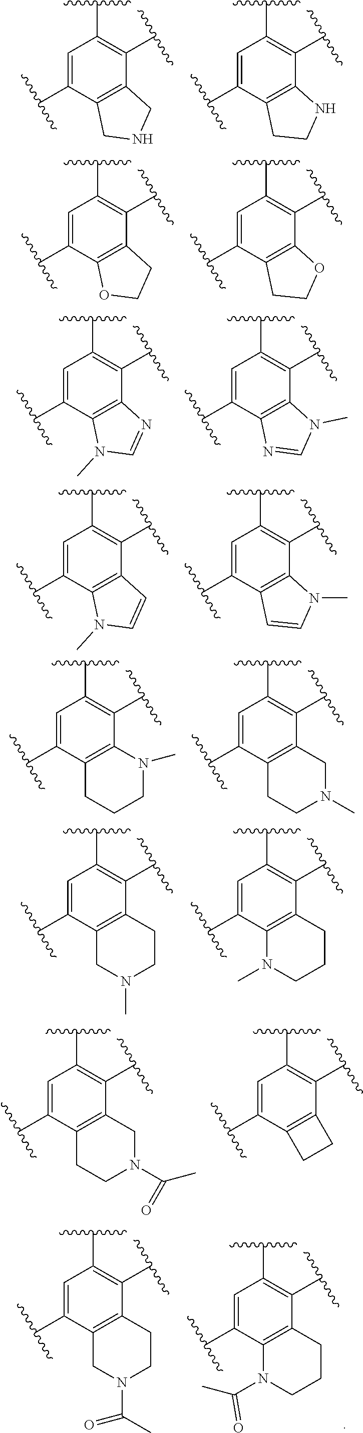

In particular, the present invention provides a compound having the following structures:

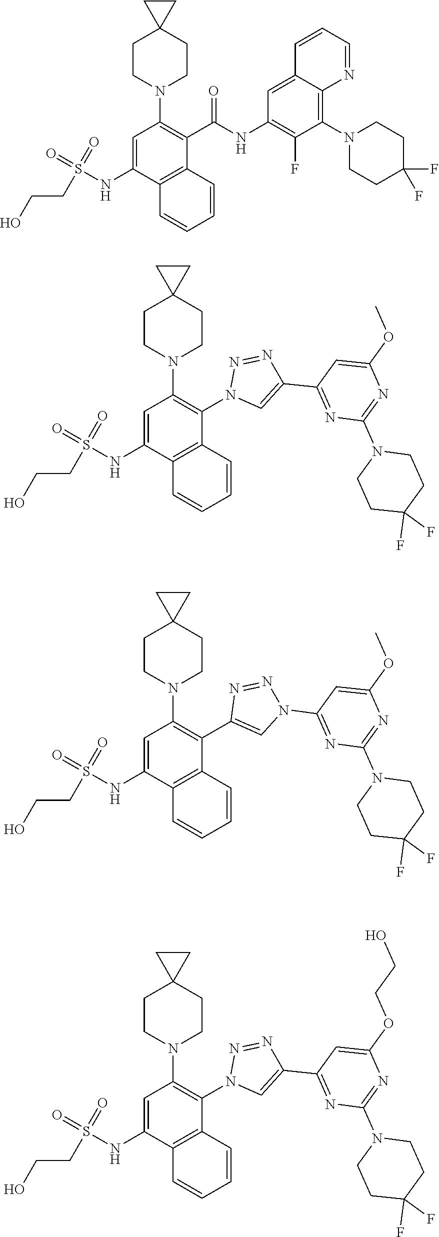

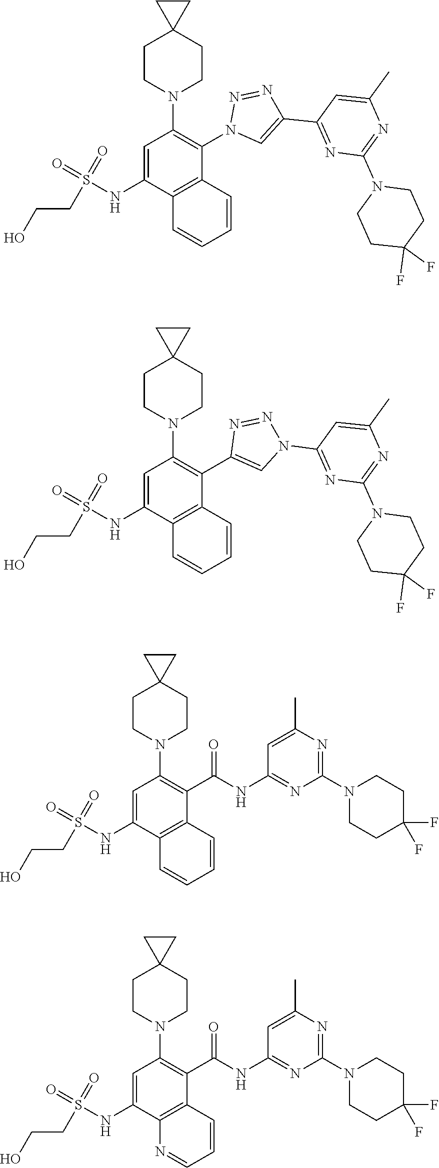 [SMILES: Cc1cc(-c2cn(-c3c(N4CCC5(CC4)CC5)cc(NS(=O)(=O)CCO)c4ccccc34)nn2)nc(N2CCC(F)(F)CC2)n1 ; Cc1cc(-n2cc(-c3c(N4CCC5(CC4)CC5)cc(NS(=O)(=O)CCO)c4ccccc34)nn2)nc(N2CCC(F)(F)CC2)n1 ; Cc1cc(NC(=O)c2c(N3CCC4(CC3)CC4)cc(NS(=O)(=O)CCO)c3ccccc23)nc(N2CCC(F)(F)CC2)n1 ; Cc1cc(NC(=O)c2c(N3CCC4(CC3)CC4)cc(NS(=O)(=O)CCO)c3ncccc23)nc(N2CCC(F)(F)CC2)n1]

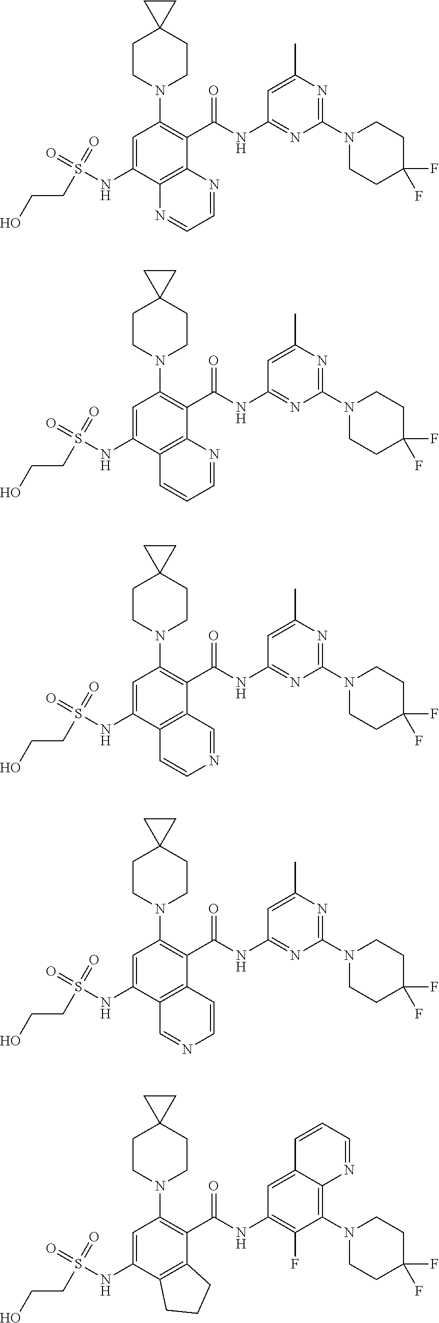

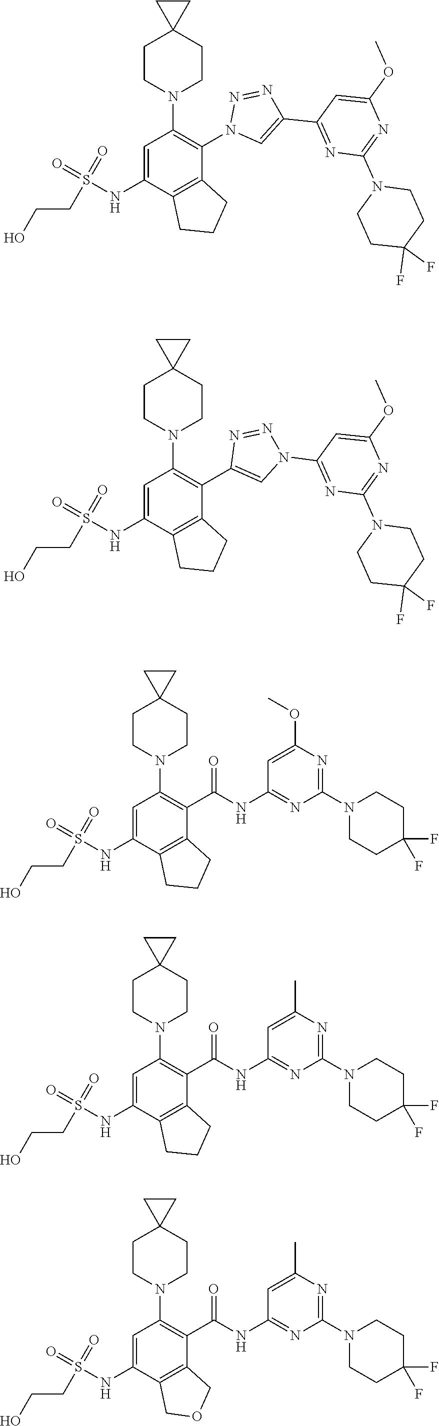 [SMILES: COc1cc(-c2cn(-c3c(N4CCC5(CC4)CC5)cc(NS(=O)(=O)CCO)c4c3CCC4)nn2)nc(N2CCC(F)(F)CC2)n1 ; COc1cc(-n2cc(-c3c(N4CCC5(CC4)CC5)cc(NS(=O)(=O)CCO)c4c3CCC4)nn2)nc(N2CCC(F)(F)CC2)n1 ; COc1cc(NC(=O)c2c(N3CCC4(CC3)CC4)cc(NS(=O)(=O)CCO)c3c2CCC3)nc(N2CCC(F)(F)CC2)n1 ; Cc1cc(NC(=O)c2c(N3CCC4(CC3)CC4)cc(NS(=O)(=O)CCO)c3c2CCC3)nc(N2CCC(F)(F)CC2)n1 ; Cc1cc(NC(=O)c2c(N3CCC4(CC3)CC4)cc(NS(=O)(=O)CCO)c3c2COC3)nc(N2CCC(F)(F)CC2)n1]

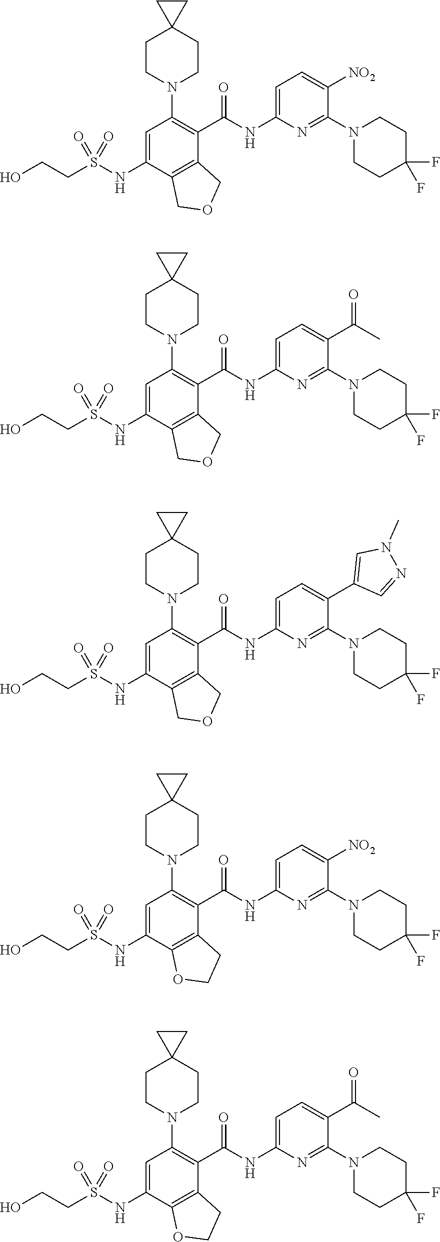 [SMILES: O=C(Nc1ccc([N+](=O)[O-])c(N2CCC(F)(F)CC2)n1)c1c(N2CCC3(CC2)CC3)cc(NS(=O)(=O)CCO)c2c1COC2 ; CC(=O)c1ccc(NC(=O)c2c(N3CCC4(CC3)CC4)cc(NS(=O)(=O)CCO)c3c2COC3)nc1N1CCC(F)(F)CC1 ; Cn1cc(-c2ccc(NC(=O)c3c(N4CCC5(CC4)CC5)cc(NS(=O)(=O)CCO)c4c3COC4)nc2N2CCC(F)(F)CC2)cn1 ; O=C(Nc1ccc([N+](=O)[O-])c(N2CCC(F)(F)CC2)n1)c1c(N2CCC3(CC2)CC3)cc(NS(=O)(=O)CCO)c2c1CCO2 ; CC(=O)c1ccc(NC(=O)c2c(N3CCC4(CC3)CC4)cc(NS(=O)(=O)CCO)c3c2CCO3)nc1N1CCC(F)(F)CC1]

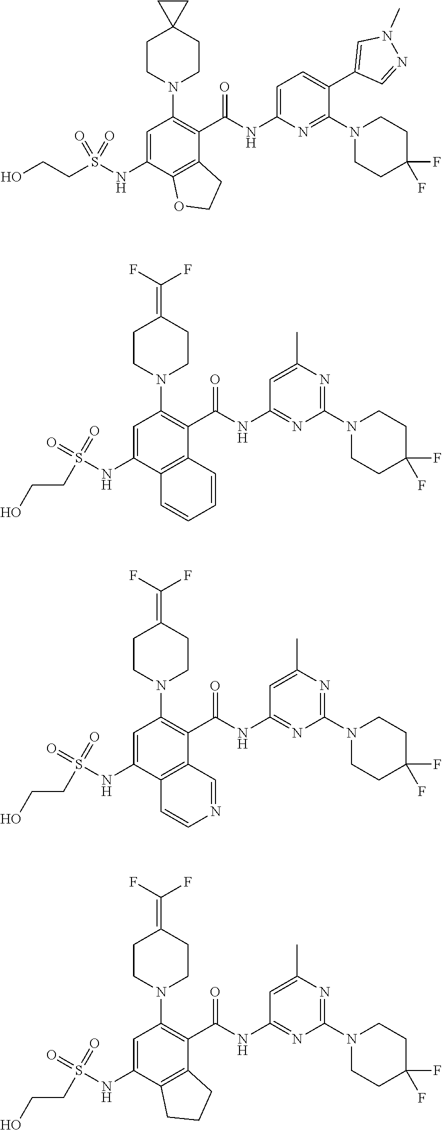 [SMILES: Cn1cc(-c2ccc(NC(=O)c3c(N4CCC5(CC4)CC5)cc(NS(=O)(=O)CCO)c4c3CCO4)nc2N2CCC(F)(F)CC2)cn1 ; Cc1cc(NC(=O)c2c(N3CCC(=C(F)F)CC3)cc(NS(=O)(=O)CCO)c3ccccc23)nc(N2CCC(F)(F)CC2)n1 ; Cc1cc(NC(=O)c2c(N3CCC(=C(F)F)CC3)cc(NS(=O)(=O)CCO)c3ccncc23)nc(N2CCC(F)(F)CC2)n1 ; Cc1cc(NC(=O)c2c(N3CCC(=C(F)F)CC3)cc(NS(=O)(=O)CCO)c3c2CCC3)nc(N2CCC(F)(F)CC2)n1]

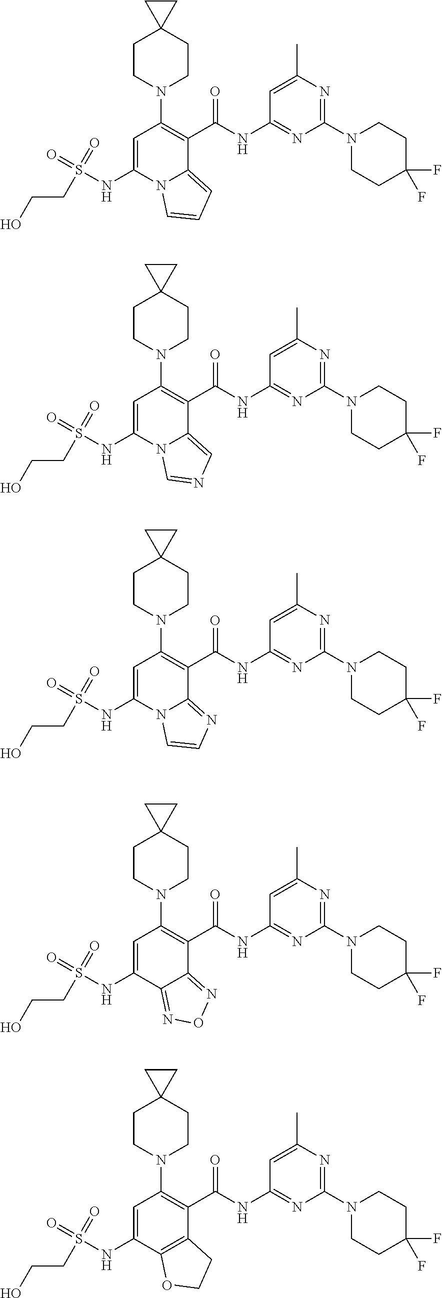 [SMILES: Cc1cc(NC(=O)c2c(N3CCC4(CC3)CC4)cc(NS(=O)(=O)CCO)n3cccc23)nc(N2CCC(F)(F)CC2)n1 ; Cc1cc(NC(=O)c2c(N3CCC4(CC3)CC4)cc(NS(=O)(=O)CCO)n3cncc23)nc(N2CCC(F)(F)CC2)n1 ; Cc1cc(NC(=O)c2c(N3CCC4(CC3)CC4)cc(NS(=O)(=O)CCO)n3ccnc23)nc(N2CCC(F)(F)CC2)n1 ; Cc1cc(NC(=O)c2c(N3CCC4(CC3)CC4)cc(NS(=O)(=O)CCO)c3nonc23)nc(N2CCC(F)(F)CC2)n1 ; Cc1cc(NC(=O)c2c(N3CCC4(CC3)CC4)cc(NS(=O)(=O)CCO)c3c2CCO3)nc(N2CCC(F)(F)CC2)n1]

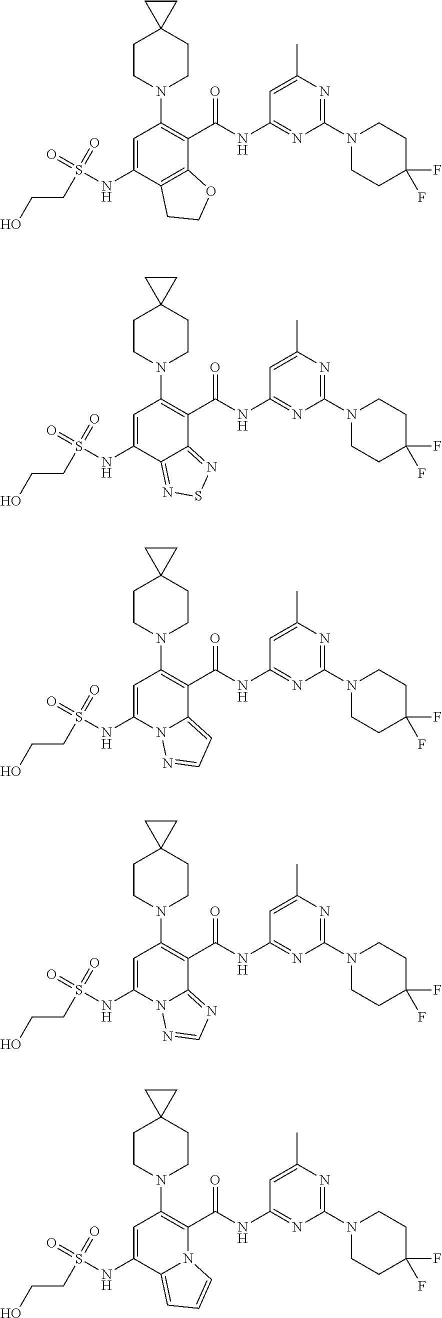 [SMILES: Cc1cc(NC(=O)c2c(N3CCC4(CC3)CC4)cc(NS(=O)(=O)CCO)c3c2OCC3)nc(N2CCC(F)(F)CC2)n1 ; Cc1cc(NC(=O)c2c(N3CCC4(CC3)CC4)cc(NS(=O)(=O)CCO)c3nsnc23)nc(N2CCC(F)(F)CC2)n1 ; Cc1cc(NC(=O)c2c(N3CCC4(CC3)CC4)cc(NS(=O)(=O)CCO)n3nccc23)nc(N2CCC(F)(F)CC2)n1 ; Cc1cc(NC(=O)c2c(N3CCC4(CC3)CC4)cc(NS(=O)(=O)CCO)n3ncnc23)nc(N2CCC(F)(F)CC2)n1 ; Cc1cc(NC(=O)c2c(N3CCC4(CC3)CC4)cc(NS(=O)(=O)CCO)c3cccn23)nc(N2CCC(F)(F)CC2)n1]

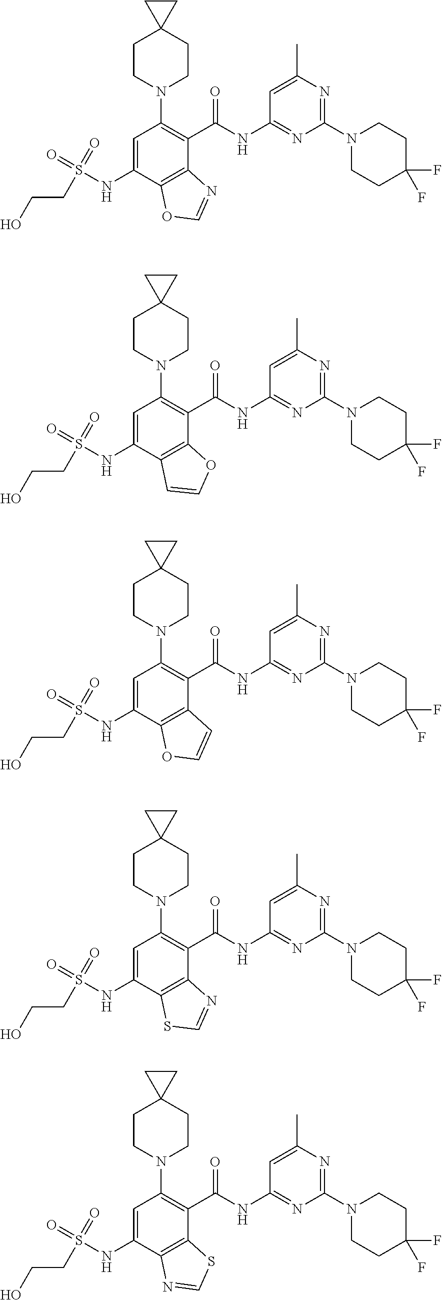 [SMILES: Cc1cc(NC(=O)c2c(N3CCC4(CC3)CC4)cc(NS(=O)(=O)CCO)c3ocnc23)nc(N2CCC(F)(F)CC2)n1 ; Cc1cc(NC(=O)c2c(N3CCC4(CC3)CC4)cc(NS(=O)(=O)CCO)c3ccoc23)nc(N2CCC(F)(F)CC2)n1 ; Cc1cc(NC(=O)c2c(N3CCC4(CC3)CC4)cc(NS(=O)(=O)CCO)c3occc23)nc(N2CCC(F)(F)CC2)n1 ; Cc1cc(NC(=O)c2c(N3CCC4(CC3)CC4)cc(NS(=O)(=O)CCO)c3scnc23)nc(N2CCC(F)(F)CC2)n1 ; Cc1cc(NC(=O)c2c(N3CCC4(CC3)CC4)cc(NS(=O)(=O)CCO)c3ncsc23)nc(N2CCC(F)(F)CC2)n1]

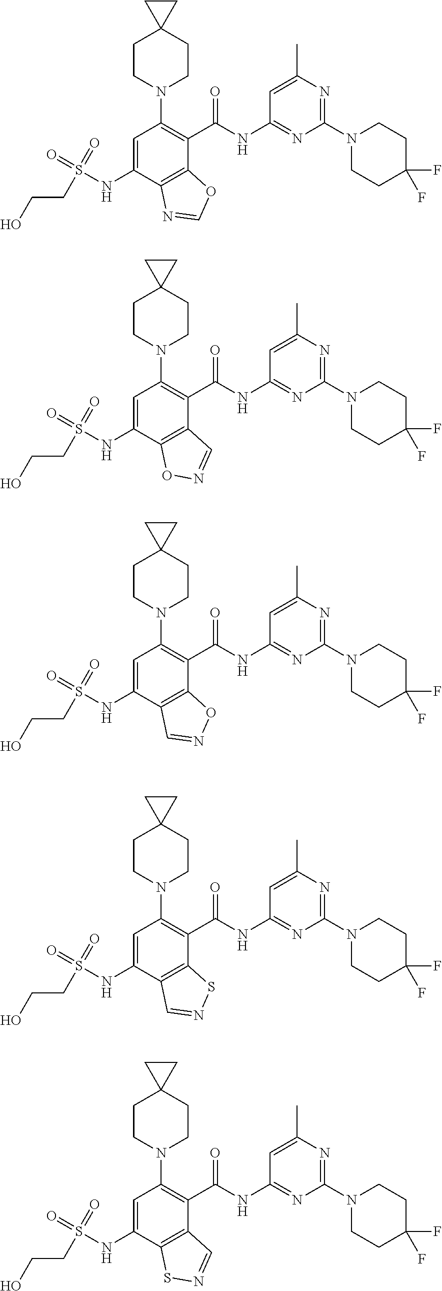 [SMILES: Cc1cc(NC(=O)c2c(N3CCC4(CC3)CC4)cc(NS(=O)(=O)CCO)c3ncoc23)nc(N2CCC(F)(F)CC2)n1 ; Cc1cc(NC(=O)c2c(N3CCC4(CC3)CC4)cc(NS(=O)(=O)CCO)c3oncc23)nc(N2CCC(F)(F)CC2)n1 ; Cc1cc(NC(=O)c2c(N3CCC4(CC3)CC4)cc(NS(=O)(=O)CCO)c3cnoc23)nc(N2CCC(F)(F)CC2)n1 ; Cc1cc(NC(=O)c2c(N3CCC4(CC3)CC4)cc(NS(=O)(=O)CCO)c3cnsc23)nc(N2CCC(F)(F)CC2)n1 ; Cc1cc(NC(=O)c2c(N3CCC4(CC3)CC4)cc(NS(=O)(=O)CCO)c3sncc23)nc(N2CCC(F)(F)CC2)n1]

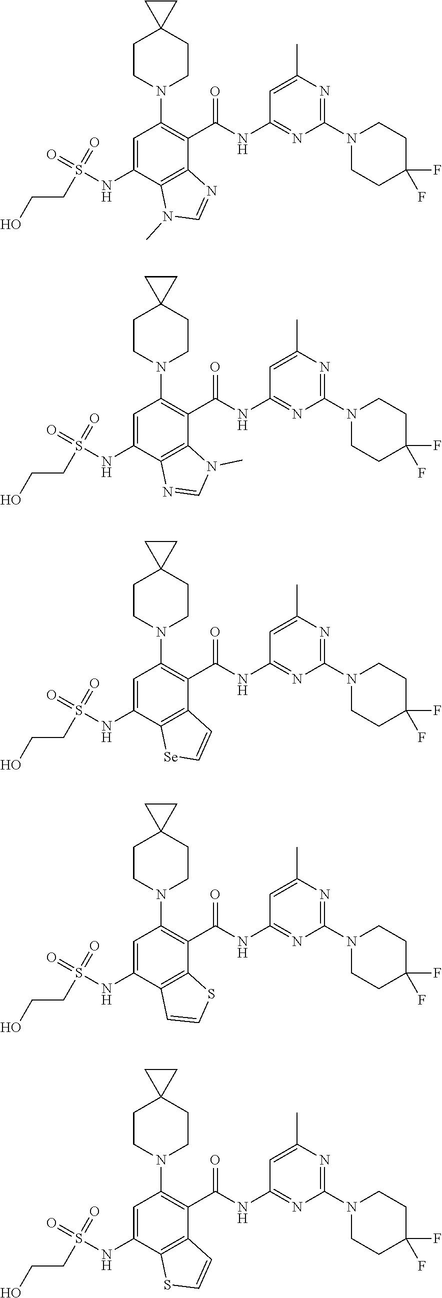

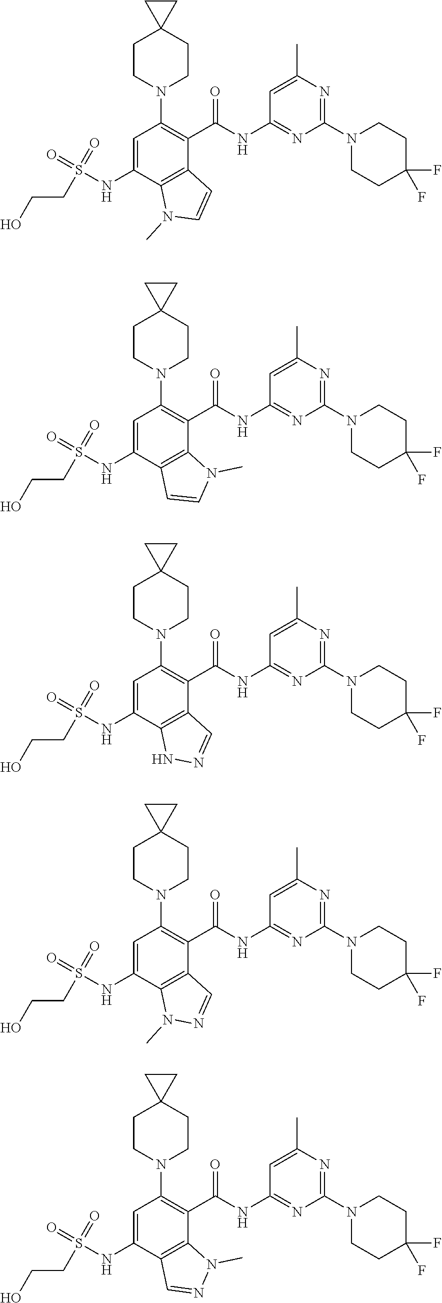

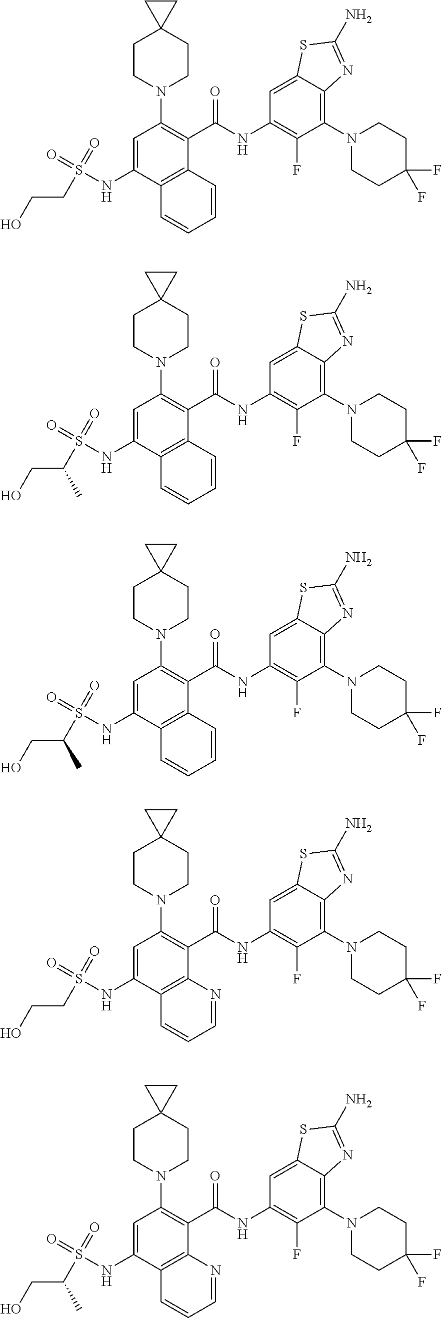

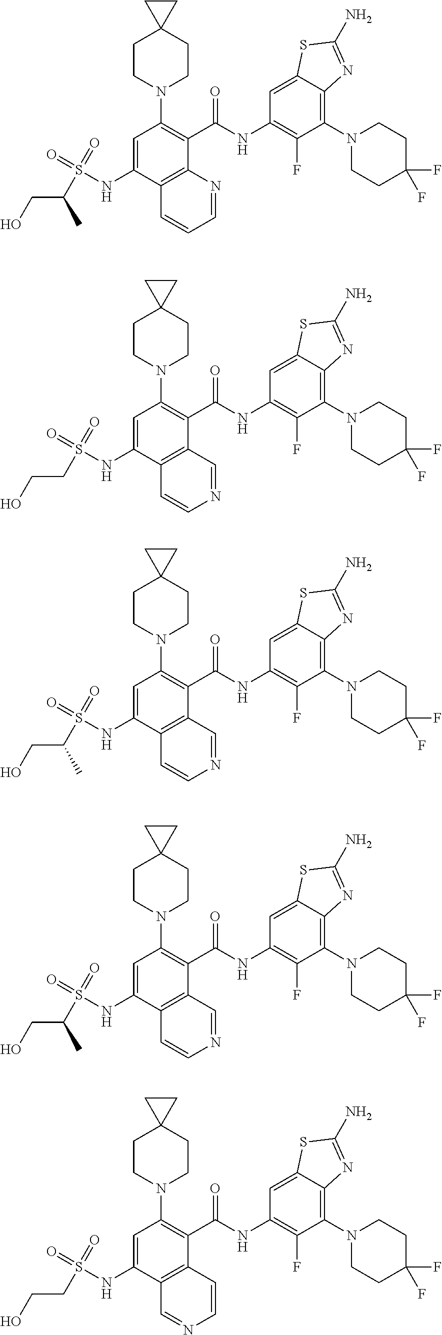

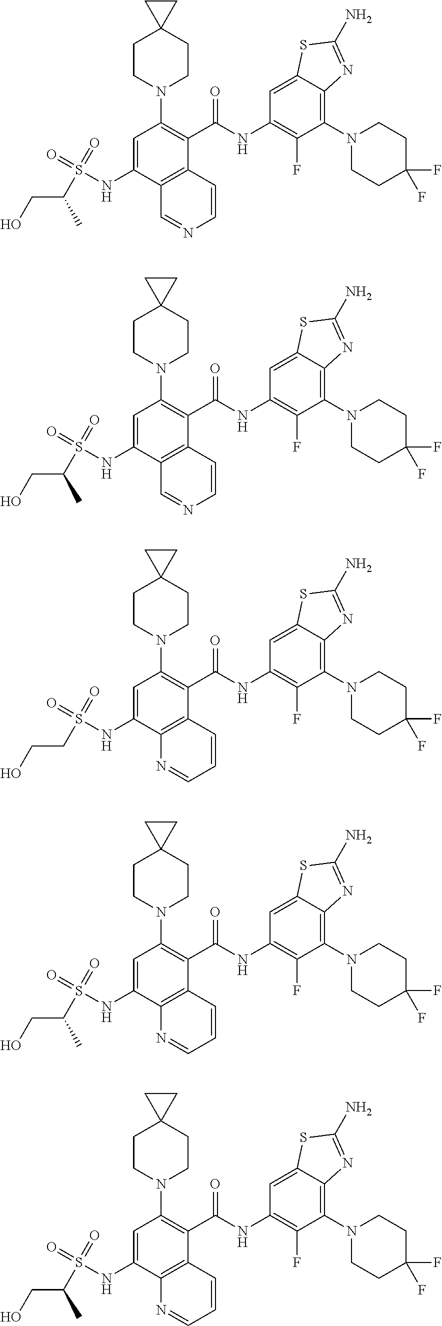

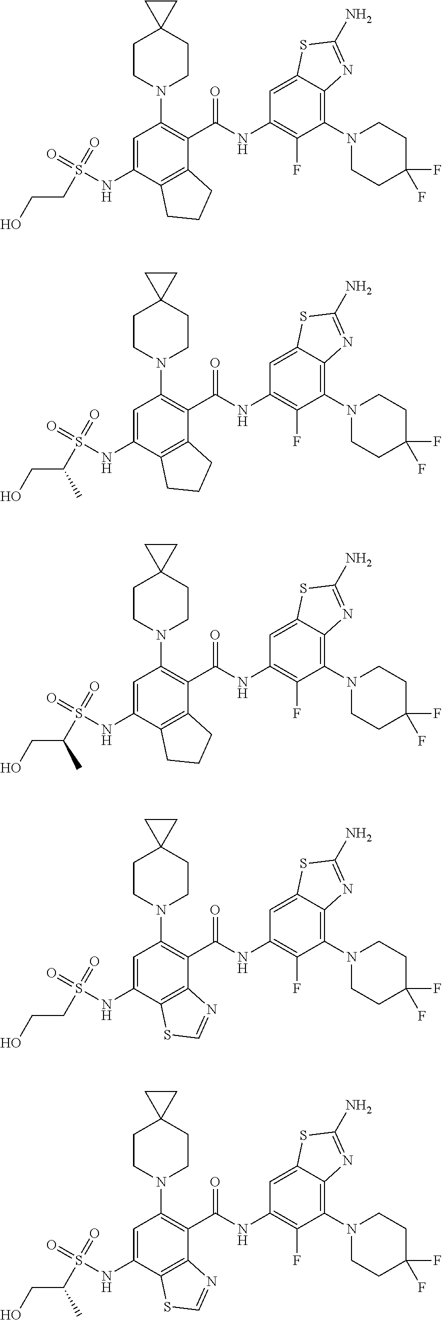 [SMILES: Nc1nc2c(N3CCC(F)(F)CC3)c(F)c(NC(=O)c3c(N4CCC5(CC4)CC5)cc(NS(=O)(=O)CCO)c4c3CCC4)cc2s1 ; C[C@H](CO)S(=O)(=O)Nc1cc(N2CCC3(CC2)CC3)c(C(=O)Nc2cc3sc(N)nc3c(N3CCC(F)(F)CC3)c2F)c2c1CCC2 ; C[C@@H](CO)S(=O)(=O)Nc1cc(N2CCC3(CC2)CC3)c(C(=O)Nc2cc3sc(N)nc3c(N3CCC(F)(F)CC3)c2F)c2c1CCC2 ; Nc1nc2c(N3CCC(F)(F)CC3)c(F)c(NC(=O)c3c(N4CCC5(CC4)CC5)cc(NS(=O)(=O)CCO)c4scnc34)cc2s1 ; C[C@H](CO)S(=O)(=O)Nc1cc(N2CCC3(CC2)CC3)c(C(=O)Nc2cc3sc(N)nc3c(N3CCC(F)(F)CC3)c2F)c2ncsc12]

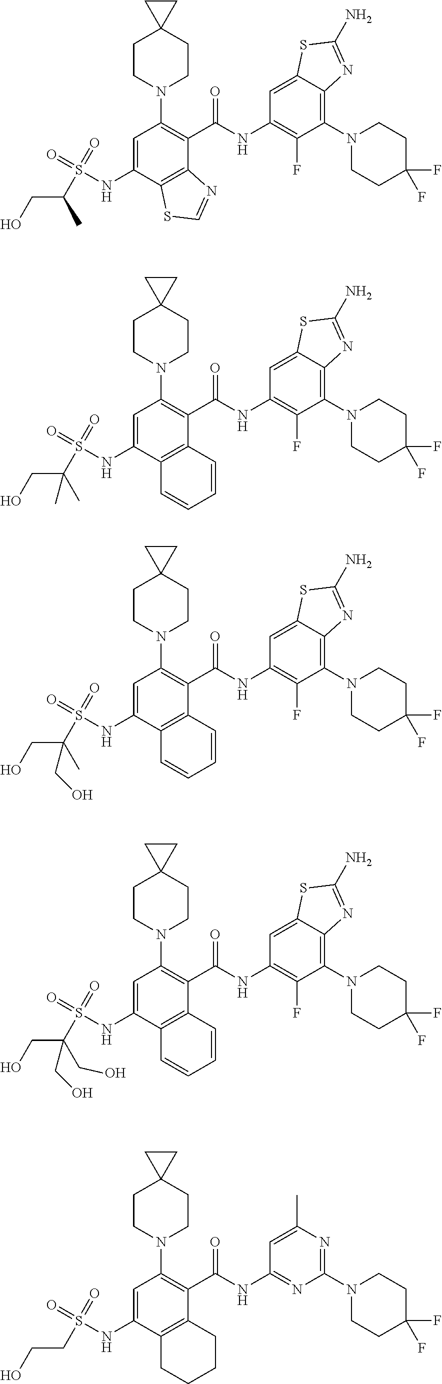

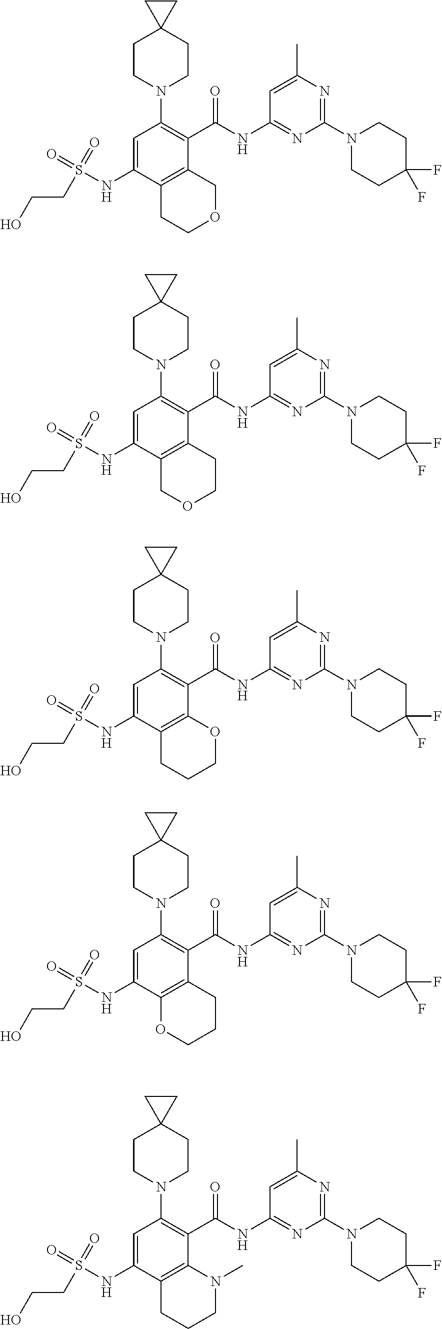

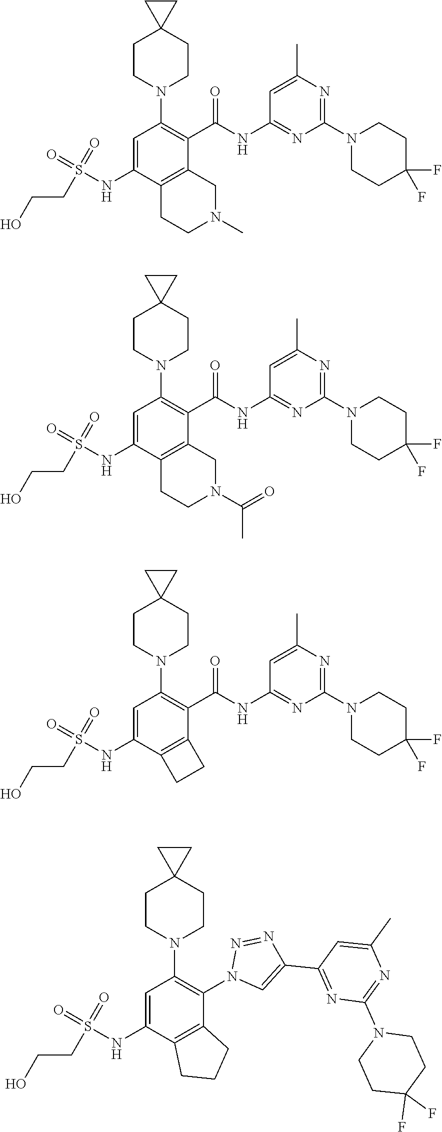

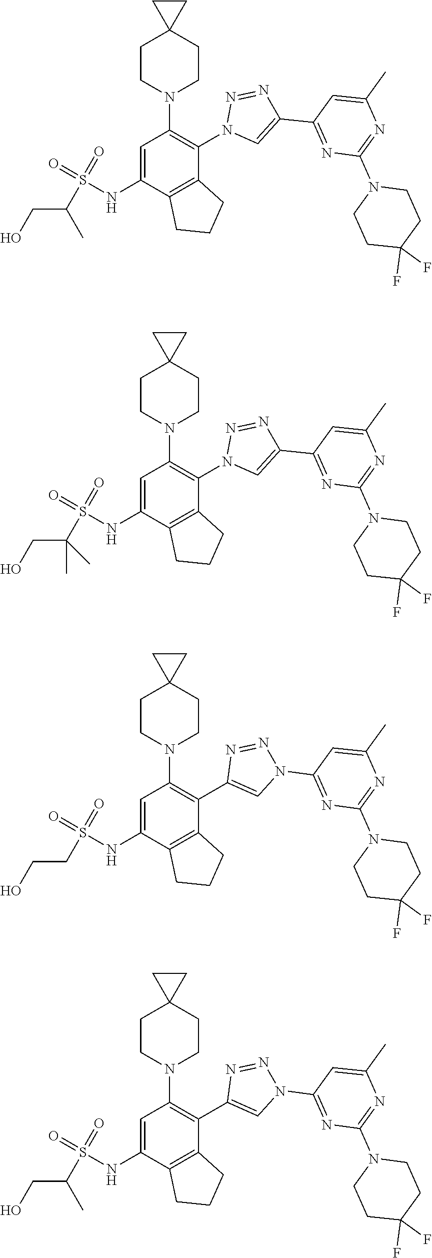 [SMILES: Cc1cc(-c2cn(-c3c(N4CCC5(CC4)CC5)cc(NS(=O)(=O)C(C)CO)c4c3CCC4)nn2)nc(N2CCC(F)(F)CC2)n1 ; Cc1cc(-c2cn(-c3c(N4CCC5(CC4)CC5)cc(NS(=O)(=O)C(C)(C)CO)c4c3CCC4)nn2)nc(N2CCC(F)(F)CC2)n1 ; Cc1cc(-n2cc(-c3c(N4CCC5(CC4)CC5)cc(NS(=O)(=O)CCO)c4c3CCC4)nn2)nc(N2CCC(F)(F)CC2)n1 ; Cc1cc(-n2cc(-c3c(N4CCC5(CC4)CC5)cc(NS(=O)(=O)C(C)CO)c4c3CCC4)nn2)nc(N2CCC(F)(F)CC2)n1]

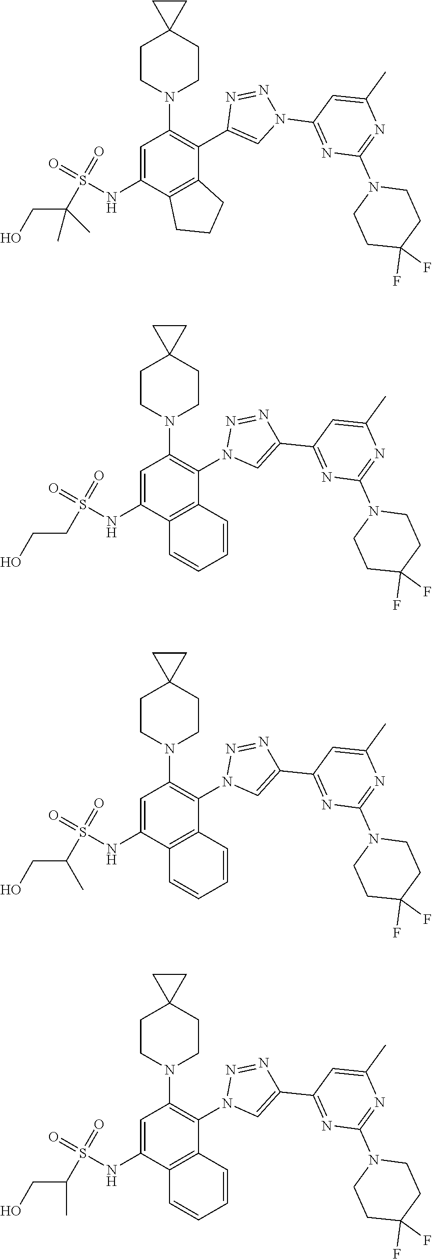 [SMILES: Cc1cc(-n2cc(-c3c(N4CCC5(CC4)CC5)cc(NS(=O)(=O)C(C)(C)CO)c4c3CCC4)nn2)nc(N2CCC(F)(F)CC2)n1 ; Cc1cc(-c2cn(-c3c(N4CCC5(CC4)CC5)cc(NS(=O)(=O)CCO)c4ccccc34)nn2)nc(N2CCC(F)(F)CC2)n1 ; Cc1cc(-c2cn(-c3c(N4CCC5(CC4)CC5)cc(NS(=O)(=O)C(C)CO)c4ccccc34)nn2)nc(N2CCC(F)(F)CC2)n1 ; Cc1cc(-c2cn(-c3c(N4CCC5(CC4)CC5)cc(NS(=O)(=O)C(C)CO)c4ccccc34)nn2)nc(N2CCC(F)(F)CC2)n1]

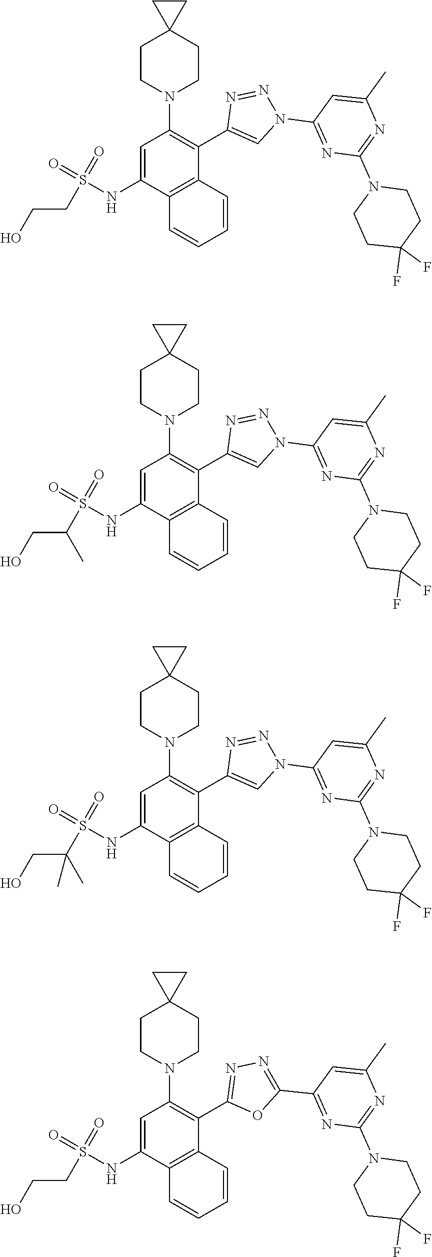 [SMILES: Cc1cc(-n2cc(-c3c(N4CCC5(CC4)CC5)cc(NS(=O)(=O)CCO)c4ccccc34)nn2)nc(N2CCC(F)(F)CC2)n1 ; Cc1cc(-n2cc(-c3c(N4CCC5(CC4)CC5)cc(NS(=O)(=O)C(C)CO)c4ccccc34)nn2)nc(N2CCC(F)(F)CC2)n1 ; Cc1cc(-n2cc(-c3c(N4CCC5(CC4)CC5)cc(NS(=O)(=O)C(C)(C)CO)c4ccccc34)nn2)nc(N2CCC(F)(F)CC2)n1 ; Cc1cc(-c2nnc(-c3c(N4CCC5(CC4)CC5)cc(NS(=O)(=O)CCO)c4ccccc34)o2)nc(N2CCC(F)(F)CC2)n1]

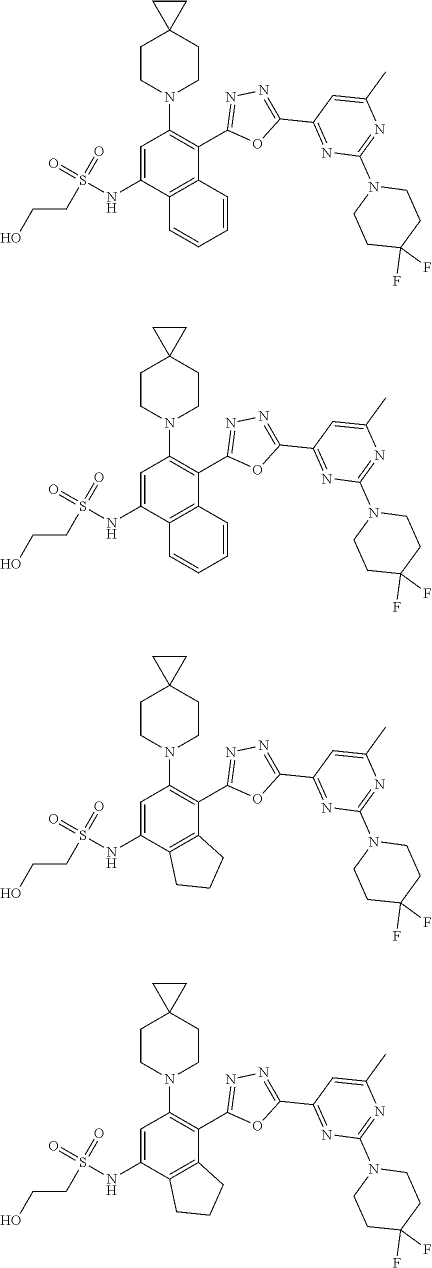 [SMILES: Cc1cc(-c2nnc(-c3c(N4CCC5(CC4)CC5)cc(NS(=O)(=O)CCO)c4ccccc34)o2)nc(N2CCC(F)(F)CC2)n1 ; Cc1cc(-c2nnc(-c3c(N4CCC5(CC4)CC5)cc(NS(=O)(=O)CCO)c4ccccc34)o2)nc(N2CCC(F)(F)CC2)n1 ; Cc1cc(-c2nnc(-c3c(N4CCC5(CC4)CC5)cc(NS(=O)(=O)CCO)c4c3CCC4)o2)nc(N2CCC(F)(F)CC2)n1 ; Cc1cc(-c2nnc(-c3c(N4CCC5(CC4)CC5)cc(NS(=O)(=O)CCO)c4c3CCC4)o2)nc(N2CCC(F)(F)CC2)n1]

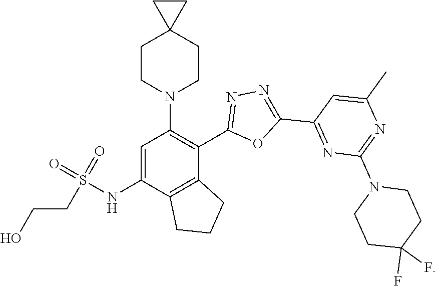 [SMILES: Cc1cc(-c2nnc(-c3c(N4CCC5(CC4)CC5)cc(NS(=O)(=O)CCO)c4c3CCC4)o2)nc(N2CCC(F)(F)CC2)n1]

In addition, the present invention provides a pharmaceutical composition that contains any one of the compounds in the Examples of the present invention or its pharmaceutically acceptable salts, stereoisomers, isotope isomers, prodrugs, hydrates or solvates and pharmaceutically acceptable carriers.

In addition, the present invention provides a method for treating tumors by inhibiting KIF18A, comprising administering any one of the compounds of the present invention or its pharmaceutically acceptable salts, stereoisomers, isotope isomers, prodrugs, hydrates or solvates to an individual in need.

### Definitions

Unless otherwise specified, the compounds of the present invention may encompass, in addition to their specific structures, their pharmaceutically acceptable salts, stereoisomers, isotope isomers (e.g., deuterated compounds), solvates, hydrates, prodrugs and metabolites. Thus, the pharmaceutically acceptable salts, stereoisomers, isotope isomers, solvates, hydrates, prodrugs, and metabolites of these compounds are also considered within the scope of protection.

Unless otherwise specified, the terms used in this patent specification and claims are defined as follows. Furthermore, many of the groups defined herein may be optionally substituted. A list of typical substituents is presented in Definitions by way of example and is not intended to limit the substituents defined elsewhere in this patent specification and claims.

The term “alkyl” refers to a linear or branched saturated aliphatic hydrocarbyl or linker, comprising 1-20 carbon atoms, preferably 1-12 carbon atoms, more preferably 1-8 carbon atoms, 1-6 carbon atoms or 1-4 carbon atoms. “Lower alkyl” specifically refers to an alkyl comprising 1-4 carbon atoms. Examples of alkyl include —(CH₂)₃—, methyl, trifluoromethyl, ethyl, propyl, isopropyl, n-butyl, isobutyl, tert-butyl and pentyl. Alkyl may be either substituted or unsubstituted. Typical substituents include cycloalkyl, aryl, heteroaryl, heterocycloalkyl, hydroxyl, alkoxy, aryloxy, mercapto, alkylthio, arylthio, cyano, halo, carbonyl, thiocarbonyl, O-carbamoyl, N-carbamoyl, O-thiocarbamoyl, N-thiocarbamoyl, C-acylamino, N-amido, C-carboxyl, O-carboxyl, nitro, silyl, amino and —NR^{x}R^{y}; wherein, R^{x} and R^{y} are independently selected from hydrogen, alkyl, cycloalkyl, aryl, carbonyl, acetyl, sulfonyl, trifluoromethanesulfonyl and a fused 5- or 6-membered heterocyclyl ring.

The term “alkenyl” refers to a linear or branched hydrocarbyl containing one or more double bonds and typically comprising 2-20 carbon atoms. For example, “C₂-C₆ alkenyl” comprises 2-6 carbon atoms. Examples of alkenyl include, but are not limited to, vinyl, propenyl, butenyl and 1-methyl-2-buten-1-yl.

The term “alkynyl” refers to a linear or branched hydrocarbyl containing one or more triple bonds and typically comprising 2-20 carbon atoms. For example, “C₂-C₆ alkynyl” comprises 2-6 carbon atoms. Examples of alkynyl include, but are not limited to, ethynyl, 1-propynyl and 1-butynyl.

The term “alkoxy” or “alkyloxy” refers to —O-alkyl. “C₁-C₆ alkoxy” (or alkyloxy) is intended to include C₁, C₂, C₃, C₄, C₅ and C₆ alkoxy. Examples of alkoxy include, but are not limited to, methoxy, ethoxy, propoxy (e.g., n-propoxy and isopropoxy) and tert-butoxy. Similarly, “alkylthio” or “thioalkoxy” refers to sulfur-bridged alkyl comprising a specified number of carbon atoms as defined above; for example, methyl-S—and ethyl-S—.

The term “cycloalkyl” refers to a 3- to 8-membered all-carbon monocyclic or bicyclic ring, a 5-/6- or 6-/6-membered fused all-carbon bicyclic ring, or a fused polycyclic ring (in a “fused” ring system, each ring shares at least one adjacent carbon atom with other rings) group, in which one or more of the rings may contain one or more double bonds but none of such rings has an intact conjugated 7r-electron system, or two rings forms a spiro by sharing one carbon. Examples of cycloalkyl include, but are not limited to, cyclopropane, cyclobutane, cyclopentane, cyclopentene, cyclohexane, cyclohexadiene, adamantane, cycloheptane and cycloheptatriene. Bicyclic alkyl includes bridged, spiro or fused-ring cycloalkyl. Illustrative examples of cycloalkyl are derived from but not limited to the following:

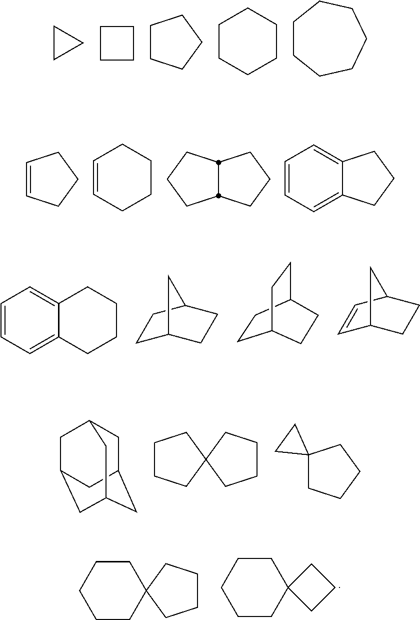

The term “aryl” refers to an all-carbon monocyclic or fused-ring polycyclic group comprising 6-12 carbon atoms, with an intact conjugated 7r-electron system. Examples of aryl include, but are not limited to, phenyl, naphthyl and anthracenyl. Aryl may be either substituted or unsubstituted. Typical substituents include halo, trihalomethyl, alkyl, hydroxyl, alkoxy, aryloxy, mercapto, alkylthio, arylthio, cyano, nitro, carbonyl, thiocarbonyl, C-carboxyl, O-carbamoyl, N-carbamoyl, O-thiocarbamoyl, N-thiocarbamoyl, C-acylamino, N-amido, sulfinyl, sulfonyl, amino and —NR^{a}R^{b}, wherein R^{a} and R^{b} are as defined above. The aryl-fused saturated or unsaturated cycloalkyl/saturated or unsaturated heterocycloalkyl may be regarded as a special substituent of aryl, and typical examples include but are not limited to:

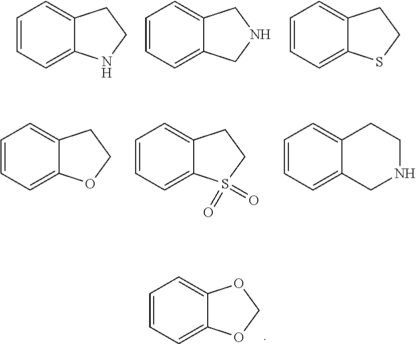

The term “heteroaryl” refers to a monocyclic or fused ring comprising 5-12 ring atoms, with one, two, three or four ring heteroatoms selected from N, O and S and the remaining ring atoms being C, having an intact conjugated 7r-electron system. Typical examples of heteroaryl include, but are not limited to, acridinyl, azetidinyl, azocinyl, benzimidazolyl, benzofuryl, benzothiofuranyl, benzothienyl, benzoxazolyl, benzoxazolinyl, benzothiazolyl, benzotriazolyl, benzotetrazolyl, benzoisoxazolyl, benzisothiazolyl, benzimidazolinyl, carbazolyl, 4aH-carbazolyl, carbolinyl, chromanyl, chromenyl, cinnolinyl, decahydroquinolyl, 2H,6H-1,5,2-dithiazinyl, dihydrofura[2,3-b]tetrahydrofuryl, furanyl, furazanyl, imidazolidinyl, imidazolinyl, imidazolyl, 1H-indazolyl, imidazopyridinyl, indolenyl, dihydroindolyl, indolizinyl, indolyl, 3H-indolyl, isatinoyl, isobenzofuranyl, isochromanyl, isoindazolyl, isoindolinyl, isoindolyl, isoquinolinyl, isothiazolyl, isothiazolopyridyl, isoxazolyl, isoxazolopyridyl, methylenedioxyphenyl, morpholinyl, diazanaphthyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridinyl, oxazolidinyl, perimidinyl, oxindolyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolopyridinyl, pyrazolyl, pyridazinyl, pyridoxazolyl, pyridinoimidazolyl, pyridothiazolyl, pyridyl, pyrimidinyl, pyrrolidinyl, pyrrolinyl, 2-pyrrolidonyl, 2H-pyrrolyl, pyrrolyl, quinazolinyl, quinolyl, 4H-quinolizinyl, quinoxalinyl, quinuclidinyl, tetrazolyl, tetrahydrofuryl, tetrahydroisoquinolyl, tetrahydroquinolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thiazolopyridyl, thienothiazolyl, thienoxazolyl, thienoimidazolyl, thienyl, triazinyl, 1,2,3-triazolyl, 1,2,4-triazolyl, 1,2,5-triazolyl, 1,3,4-triazolyl and xanthyl, quinolyl, isoquinolinyl, phthalazinyl, quinazolinyl, indolyl, isoindolyl, dihydroindolyl, 1H-indazolyl, benzimidazolyl, 1,2,3,4-tetrahydroquinolyl, 1,2,3,4-tetrahydroisoquinolyl, 5,6,7,8-tetrahydro-quinolyl, 2,3-dihydro-benzofuranyl, chromanyl, 1,2,3,4-tetrahydro-quinoxalinyl and 1,2,3,4-tetrahydro-quinazolinyl. The term “heteroaryl” may also include biaryl structures formed by the above-defined “aryl” and monocyclic “heteroaryl”, including but not limited to “-phenylbipyridyl-”, “-phenylbipyrimidinyl”, “-pyridylbiphenyl”, “-pyridylbipyrimidinyl-” and “-pyrimidinylbiphenyl-”; wherein the present invention also includes fused ring and spiro ring compounds containing, for example, heterocyclic rings mentioned above.

A pharmaceutically acceptable heteroaryl is sufficiently stable to be linked to a compound of the present invention for formulation into a pharmaceutical composition and subsequent administration to patients in need.

Unless otherwise defined, the definitions of substituents in the present invention are independent and not interrelated. For example, the definition of R^{a} (or R^{b}) is independent across different substituents. Specifically, when one definition is selected for R^{a} (or R^{b}) in a substituent, it does not imply that the same definition applies to R^{a} (or R^{b}) in other substituents. More specifically, when R^{a} (or R^{b}) is defined as hydrogen in NR^{a}R^{b}, for example (non-exhaustive), it does not imply that R^{a} (or R^{b}) in —C(O)—NR^{a}R^{b} must be hydrogen.

“Halo” or “halogen” includes fluorine, chlorine, bromine and iodine. “Haloalkyl” is intended to include branched and linear saturated aliphatic hydrocarbyl having a specified number of carbon atoms substituted with 1 or more halogens. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl and heptachloropropyl. Examples of haloalkyl also include “fluoroalkyl” which is intended to include branched and linear saturated aliphatic hydrocarbyl having a specified number of carbon atoms and substituted with 1 or more fluorine atoms.

“Haloalkoxy” or “haloalkyloxy” refers to oxygen-bridged haloalkyl having a specified number of carbon atoms as defined above. For example, “C₁-C₆ haloalkoxy” is intended to include C₁, C₂, C₃, C₄, C₅ and C₆ haloalkoxy. Examples of haloalkoxy include, but are not limited to, trifluoromethoxy, 2,2,2-trifluoroethoxy and pentafluoroethoxy. Similarly, “haloalkylthio” or “thiohaloalkoxy” refers to sulfur-bridged haloalkyl comprising a specified number of carbon atoms as defined above; for example, trifluoromethyl-S—and pentafluoroethyl-S—.

In the present disclosure, the expression Cx1-Cx2 is used when referring to some substituents, which means that the number of carbon atoms in the substituent may be x1 to x2. For example, C₀-C₈ means that the group contains 0, 1, 2, 3, 4, 5, 6, 7 or 8 carbon atoms, C₁-C₈ means that the group contains 1, 2, 3, 4, 5, 6, 7 or 8 carbon atoms, C₂-C₈ means that the group contains 2, 3, 4, 5, 6, 7 or 8 carbon atoms, C₃-C₈ means that the group contains 3, 4, 5, 6, 7 or 8 carbon atoms, C₄-C₈ means that the group contains 4, 5, 6, 7 or 8 carbon atoms, C₀-C₆ means that the group contains 0, 1, 2, 3, 4, 5 or 6 carbon atoms, C₁-C₆ means that the group contains 1, 2, 3, 4, 5 or 6 carbon atoms, C₂-C₆ means that the group contains 2, 3, 4, 5 or 6 carbon atoms, and C₃-C₆ means that the group contains 3, 4, 5 or 6 carbon atoms.

In the present disclosure, the expression “x1-x2-membered ring” is used when referring to cyclic groups (e.g., aryl, heteroaryl, cycloalkyl and heterocycloalkyl), which means that the number of ring atoms in the group may be x1 to x2. For example, 3-12-membered cyclic group may be a 3, 4, 5, 6, 7, 8, 9, 10, 11 or 12-membered ring comprising 3, 4, 5, 6, 7, 8, 9, 10, 11 or 12 ring atoms; 3-6-membered cyclic group may be a 3, 4, 5 or 6-membered ring comprising 3, 4, 5 or 6 ring atoms; 3-8-membered cyclic group may be a 3, 4, 5, 6, 7 or 8-membered ring comprising 3, 4, 5, 6, 7 or 8 ring atoms; 3-9-membered cyclic group may be a 3, 4, 5, 6, 7, 8 or 9-membered ring comprising 3, 4, 5, 6, 7, 8 or 9 ring atoms; 4-7-membered cyclic group may be a 4, 5, 6 or 7-membered ring comprising 4, 5, 6 or 7 ring atoms; 5-8-membered cyclic group may be a 5, 6, 7 or 8-membered ring comprising 5, 6, 7 or 8 ring atoms; 5-12-membered cyclic group may be a 5, 6, 7, 8, 9, 10, 11 or 12-membered ring comprising 5, 6, 7, 8, 9, 10, 11 or 12 ring atoms; 6-12-membered cyclic group may be a 6, 7, 8, 9, 10, 11 or 12-membered ring comprising 6, 7, 8, 9, 10, 11 or 12 ring atoms. The ring atoms may be carbon atoms or heteroatoms, such as heteroatoms selected from N, O and S. The heterocyclic rings may contain 1, 2, 3, 4, 5, 6, 7, 8, 9, 10 or more ring heteroatoms, such as heteroatoms selected from N, O and S.

In the present invention, one or more halogens may be each independently selected from fluorine, chlorine, bromine and iodine.

The term “substituted” as used herein means that at least one hydrogen atom is substituted by a non-hydrogen group, provided that normal valence is maintained and the substitution results in a stable compound. The term “cyclic double bond” as used herein refers to a double bond formed between two adjacent ring atoms (e.g. C═C, C═N or N═N).

Where nitrogen atoms (e.g. amines) are present on the compounds of the invention, these nitrogen atoms may be converted to N-oxides by treatment with an oxidizing agent (e.g. mCPBA and/or hydrogen peroxide) to obtain other compounds of the invention. Therefore, the nitrogen atoms shown and claimed are deemed to encompass the nitrogen atoms shown and their N-oxides to obtain derivatives of the present invention.

When any variable occurs more than once in any composition or formula of a compound, its definition in each occurrence is independent of its definition in every other occurrence Therefore, for example, if a group is shown substituted with 0-3 Rs, the group may be optionally substituted with up to three R groups, with R defined independently in each occurrence. Furthermore, combinations of substituents and/or variables are permitted only if such combinations result in stable compounds.

The term “patient” as used herein refers to an organism to be treated by a method of the present invention. Such organisms preferably include, but are not limited to, mammals (e.g. rodents, apes/monkeys, horses, cattle, pigs, dogs and cats) and most preferably humans.

The term “effective amount” as used herein refers to an amount of a drug or agent (i.e., a compound of the invention) that will elicit, for example, a biological or medical response in a tissue, a system, an animal or a human sought by a researcher or clinician. Furthermore, the term “therapeutically effective amount” refers to an amount that results in improved treatment, cure, prevention or alleviation of a disease, condition or side effect, or reduction in the progression rate of a disease or condition, as compared to a corresponding subject who does not receive such amount. An effective amount may be administered in one or more doses and is not intended to be limited to a particular formulation or route of administration. The term also includes an amount effective to enhance normal physiological functions within its range.

The term “treatment” as used herein includes its broad sense and encompasses both therapeutic and/or prophylactic treatment of an object. Specifically, “treatment” includes any treatment that results in the alleviation, inhibition, elimination and relief and/or prevention of a condition, disease and disorder, such as alleviation, reduction, regulation, relief, elimination, prophylaxis, prevention or remission of symptoms thereof. The therapeutic treatment includes alleviating, inhibiting or relieving symptoms or conditions of a disease; inhibiting the occurrence of complications; alleviating the underlying metabolic syndrome; inhibiting the occurrence of a disease or condition such as controlling the progression of the disease or condition; alleviating a disease or condition; reducing a disease or condition; alleviating complications caused by a disease or condition, or treating signs caused by a disease or condition. The prophylactic treatment includes a prior treatment to prevent, block or delay, slow the onset or progression of a disease or condition or reduce the severity of the disease or condition.

Similarly, the term “therapeutic agent” also includes agents or reagents used in the therapeutic and/or prophylactic treatment of an object.

The terms “pharmaceutical” or “pharmaceutically acceptable” as used herein refer to compounds, substances, compositions and/or dosage forms that, within the scope of sound medical judgment, are suitable for contact with human and animal tissues without excessive toxicity, irritation, allergic reactions and/or other issues or complications, and commensurate with a reasonable benefit/risk ratio.

### Specific Pharmaceutical and Medical Terms

The term “cancer” as used herein refers to an abnormal growth of cells that is uncontrollable and, under certain conditions, capable of metastasis (spread). This type of cancer includes, but is not limited to, solid tumors [such as bladder, intestine, brain, chest, uterus, heart, kidney, lung, lymphoid tissue (lymphoma), ovary, pancreas or other endocrine organs (e.g. thyroid gland), prostate and skin (melanoma)] or blood tumors (e.g. non-leukemic leukemia).

The term “combined administration” or similar terms thereof, as used herein, refers to the administration of several selected therapeutic agents to a patient in the same or different modes of administration and at the same or different times.

The term “enhancement” or “capable of enhancing” as used herein refers to an expected outcome of an increase or extension in either potency or duration. Thus, in the context of enhancing the therapeutic effect of a drug, the term “capable of enhancing” refers to the ability of the drug to increase or extend its potency or duration within the system. The term “enhancement value” as used herein refers to the ability to maximize the efficacy of another therapeutic agent in an ideal system.

The term “immune disease” refers to a disease or condition resulting from an adverse or harmful response to endogenous or exogenous antigens, typically leading to cellular dysfunction, or functional damage and impairment, or damage to organs or tissues that may produce immune symptoms.

The term “kit” is synonymous with “product packaging.”

The terms “object”, “subject” or “patient” include both mammals and non-mammals. Mammals include, but are not limited to, mammals including humans and non-human primates (such as orangutans, apes and monkeys), agricultural animals (such as cattle, horses, goats, sheep and pigs), domestic animals (such as rabbits and dogs), and laboratory animals including rodents (such as rats, mice and guinea pigs). Non-mammals include, but are not limited to, birds and fish. In a preferred Example, the selected mammal is human.

As used herein, a compound or pharmaceutical composition, after administered, can relieve a disease, symptom or condition, particularly to reduce severity, delay onset, slow down progression or shorten duration. This applies whether the administration is fixed or temporary, continuous or intermittent, and can be attributed to or associated with the administration.

### Routes of Administration

Suitable routes of administration include, but are not limited to, oral, intravenous, rectal, aerosol, parenteral, ocular, pulmonary, transdermal, vaginal, ear canal, nasal and topical. In addition, by way of example only, routes of parenteral administration include intramuscular, subcutaneous, intravenous, intramedullary, ventricular, intraperitoneal, intralymphatic and intranasal.

The mode of administration of the compounds of the present invention may be topical. In certain Examples, long-acting formulations are administered by (subcutaneous or intramuscular) implantation or intramuscular injection. In another Example, administration is achieved by a targeted drug delivery system. For example, liposomes coated with organ-specific antibodies. In this Example, the liposomes are selectively directed to and absorbed by specific organs.

### Pharmaceutical Composition and Dosage

The term “pharmaceutical carrier” as used herein refers to a pharmaceutical substance, composition or vehicle, such as a liquid or solid filler, a diluent, an excipient, a manufacturing aid (e.g., lubricant, talc, magnesium stearate, calcium stearate or zinc stearate or stearic acid) or a solvent-encapsulated substance, used to carry or deliver the target compound from one organ or part of the body to another. Each carrier must be “acceptable” in the sense of being compatible with other ingredients of the formulation and harmless to patients.

The term “pharmaceutical composition” refers to a composition comprising a compound of the present invention and optionally a pharmaceutical carrier. The “pharmaceutical carrier” refers to a medium generally accepted in the art for delivering a biologically active agent to animals (particularly mammals), including (i.e.) adjuvants, excipients or vehicles such as diluents, preservatives, fillers, flow regulators, disintegrants, wetting agents, emulsifiers, suspending agents, sweeteners, corrigents, perfuming agents, antibacterial agents, antifungal agents, lubricants and dispersants, depending on the mode of administration and nature of the dosage form.

The pharmaceutical composition of the present invention may comprise a therapeutically effective amount of one or more compounds described in the present invention formulated with optionally one or more pharmaceutical carriers (additives) and/or diluents, and optionally one or more other therapeutic agents. The compounds of the present invention may be administered by any suitable means for any of the above uses, for example orally, such as in the form of tablets, pills, powders, granules, elixirs, tinctures, suspensions (including nanosuspensions, microsuspensions and spray-dried dispersions), syrups and emulsions; sublingually; buccally; parenterally, such as by subcutaneous, intravenous, intramuscular or intrasternal injection or infusion techniques (e.g. in the form of sterile injectable aqueous or non-aqueous solutions or suspensions); nasally, including administering to the nasal membrane, such as by inhalation spray; topically, such as in the form of creams or ointments; rectally, such as in the form of suppositories; or intratumorally. They may be administered alone; however, they are typically administered via a drug carrier selected based on the chosen route of administration and standard pharmaceutical practice.

Pharmaceutical carriers are formulated based on various factors known to those skilled in the art. These factors include, but are not limited to: the type and nature of the active agent formulated; the subject to whom the composition containing the active agent is to be administered; the intended route of administration of the composition; and the targeted therapeutic indication. Pharmaceutical carriers include aqueous and non-aqueous liquid media and various solid and semisolid dosage forms.

The above-mentioned carriers may comprise many different ingredients and additives in addition to the active agent. These ingredients are included in the formulation for various reasons known to those skilled in the art, such as stabilizing active agents and binding agents. Descriptions of suitable pharmaceutical carriers and factors involved in carrier selection can be obtained from several readily available sources, such as Allen L. V. Jr. et al. Remington: The Science and Practice of Pharmacy (2 Volumes), 22nd Edition (2012), Pharmaceutical Press.

Dosage regimens of the compounds of the present invention will, of course, vary depending on known factors such as the pharmacodynamic properties of the particular agent and its mode and route of administration; the species, age, sex, health status, medical condition and weight of the recipient; the nature and severity of symptoms; the type of concurrent treatment; the frequency of treatment; the route of administration, renal and hepatic function of the patient and expected effects. According to general guidance, the daily oral dose of each active ingredient when used for a given effect should range from about 0.001 mg/day to 10-5000 mg/day, preferably from about 0.01 mg/day to 1000 mg/day, and most preferably from about 0.1 mg/day to 250 mg/day. During constant-rate infusion, the most preferred intravenous dose should range from about 0.01 mg/kg/min to 10 mg/kg/min. The compounds of the present invention may be administered in a single daily dose, or divided into two, three or four doses daily.

The compounds are typically administered in the form of mixtures with suitable pharmaceutical diluents, excipients or carriers (collectively referred to herein as pharmaceutical carriers) appropriately selected according to the intended form of administration (e.g., oral tablets, capsules, elixirs and syrups) and conventional pharmaceutical practice.

A dosage form (pharmaceutical composition) suitable for administration may contain about 1 mg-2000 mg of active ingredient per dosage unit. In any of these pharmaceutical compositions, the active ingredient will generally be present in an amount of about 0.1-95% by weight based on the total weight of the composition.

The scope of the present invention includes pharmaceutical compositions comprising a therapeutically effective amount of at least one compound of the invention as an active ingredient, alone or in combination with a pharmaceutical carrier. Optionally, the compounds of the invention may be used alone, in combination with other compounds of the invention or in combination with one or more other therapeutic agents (e.g. anticancer agents or other pharmaceutically active substances).

The compounds of the present invention (which may be used in a suitable hydrated form) and/or the pharmaceutical compositions of the present invention are formulated into pharmaceutical dosage forms by conventional methods known to those skilled in the art, regardless of the selected route of administration.

The actual dose level of the active ingredient in a pharmaceutical composition of the present invention may be adjusted to reach an amount effective for achieving the desired therapeutic response, composition and mode of administration and ensuring nontoxicity for a particular patient.

The dosage level will be selected depending on a variety of factors, including the activity of the particular compound of the invention or its ester, salt or amide; route of administration; duration of administration; the excretion rate of the particular compound; the rate and extent of absorption; duration of treatment; other medications, compounds and/or substances used in combination with the particular compound; factors well known in the medical art such as age, sex, weight, condition, general health and previous medical history of the patient to be treated.

A physician or veterinarian of ordinary skill in the art can readily determine and prescribe an effective amount of a desired pharmaceutical composition. For example, to achieve a desired therapeutic effect, a physician or veterinarian may start with a dose level lower than the desired level for any compound of the present invention incorporated in a pharmaceutical composition, and then gradually increase the dose level until the desired effect is achieved. Typically, a suitable daily dose for a compound of the invention will be the lowest dose of the compound effective to produce a therapeutic effect. This effective dose usually depends on the factors mentioned above. Typically, oral, intravenous, intracerebroventricular and subcutaneous doses for compounds of the invention range from about 0.01 to 50 mg/kg body weight per day for use in patients. If desired, an effective daily dose of the active compound may be administered in two, three, four, five, six or more sub-doses at a suitable interval throughout the day, optionally in unit dosage form. In certain Examples of the present invention, administration is once daily.

Although the compounds of the present invention may be administered alone, they are preferably administered in the form of pharmaceutical formulations (compositions).

### Kit/Product Packaging

For use in the treatment of the above indications, kit/product packaging is also described herein. A kit may consist of a delivery device, a drug pack, or a container box which may be divided into several compartments to accommodate one or more containers such as vials, tubes and the like, each containing a separate ingredient involved in a method described herein. Suitable containers include bottles, vials, syringes and test tubes. These containers are made of acceptable materials such as glass or plastic.

For example, a container may contain one or more compounds described herein, either as a pharmaceutical ingredient or in a mixture with other ingredients described herein. The container may have a sterile outlet (e.g. the container may be an intravenous infusion bag or bottle, and its stopper can be pierced by a hypodermic needle). A kit may contain a compound and a description, label or instructions for use for a method described herein.

A typical kit may comprise one or more containers, each containing one or more materials (e.g. reagents, concentrated stock solutions and/or devices) to accommodate commercial promotion and user needs for the use of the compound. These materials include, but are not limited to, buffers, diluents, filters, needles, syringes, delivery devices, bags, containers, bottles and/or test tubes, accompanied by a list of contents and/or instructions for use, and a description of built-in packaging (if any). The entire set of instructions should be included in the kit.

A label may appear on or closely associated with a container. The presence of a label on a container means that letters, numbers or other features are pasted, molded and engraved onto the container; the label may also appear inside a container box or transport box containing multiple containers, such as in a product insert. A label may be used to indicate a specific therapeutic use of the contents. The label may also carry instructions for the use of the contents, such as described in the methods above.

All features described in the specification (including any stated claim, abstract and figure) and/or all steps involved in any method or process may exist in any combination unless some features or steps are mutually exclusive in the same combination.

The above-mentioned features in the present invention or the features mentioned in Examples may be combined arbitrarily. All features disclosed in the specification may be used in conjunction with any composition form. Each feature disclosed in the specification may be replaced by any alternative feature that provides the same, equivalent or similar purpose. Therefore, unless otherwise specified, the features disclosed are only general examples of equivalent or similar features.

The present invention will be further described below in conjunction with specific Examples. It should be noted that these Examples are not intended to define the scope of the present invention but merely to describe the present invention. The experimental methods with no specific conditions indicated in the following Examples usually follow conventional conditions or the conditions recommended by manufacturers. Unless otherwise stated, all percentages, ratios, proportions or parts are measured by weight.

The unit of weight-to-volume percentage in the present invention is well known to those skilled in the art, for example, it refers to the weight of solute in 100 mL of solution. Unless otherwise defined, all technical and scientific terms used herein have the same meanings as those familiar to those skilled in the art. In addition, any methods and materials similar or equivalent to those described herein may be used in the methods of the present invention. The preferred methods and materials described herein are for exemplary purposes only.

### DETAILED DESCRIPTION OF THE EMBODIMENTS

The present invention is further described through the following specific Examples, which are not intended to limit the scope of the present invention to these Examples. Experimental methods with no specific conditions indicated in the following Examples should be selected according to conventional methods and conditions, or commercial specifications.

NMR is measured using Bruker AVANCE-400 NMR spectrometer. Solvents used for measurement are indicated in the spectrum analysis.

MS measurement is conducted using Agilent 1200-G1956A/1200-6110A/1200-6140A/1260-6125B/Prime-6125B/1260-6120 liquid chromatograph-mass spectrometer (LC/MS), SHIMADZU 20A-2010/20A-2020 LC/MS and Waters ACQ-QDA LC/MS.

HPLC analysis is performed using a SHIMADZU 20A high performance liquid chromatograph.

SFC analysis and determination are conducted using Waters UPCC with PDA Detector and QDa Detector ultra-high performance convergence chromatograph, Waters UPC² with PDA detector ultra-high performance convergence chromatograph, Agilent 1260 with DAD detector high-performance liquid chromatograph, Shimadzu LC-20AB with PDA detector high-performance liquid chromatograph and Shimadzu LC-20AD with PDA detector high-performance liquid chromatograph.

Preparative HPLC separation is conducted using Shimadzu LC-20AP pump, Shimadzu LH-40 Liquid Handler, Shimadzu SPD-20A Detector, Gilson GX-281 Liquid Handler, Gilson 322 pump and Gilson 156 UV Detector preparative chromatographs.

SFC separation is conducted using The Berger MG II, MG III, Sepiatec's Prep SFC 100 system, Waters Prep 80Q SFC SYSTEM, Prep 150 AP SFC SYSTEM, Prep 200 SFC SYSTEM and Prep 350 SFC SYSTEM.

Flash column chromatography separation is conducted using Biotage IsoleraOne flash-preparative chromatograph.

The GF254 acrylic adhesive silica gel plate manufactured by Anhui Liangchen Silicon Material Co., Ltd. is used for thin-layer chromatography (TLC). The specification of the TLC silica gel plate is 0.25 mm and that for products separated and purified by TLC is 0.5 mm.

Pressurized hydrogenation reaction is conducted using hydrogenation bottles and hydrogen gas cylinders.

Microwave reaction is conducted using Biotage Initiator+microwave synthesizer.

The glove box used is customized by DELLIX.

The present invention is further described through the following Examples, which are not intended to impose any limitations on the present invention. The compounds of the present invention can be prepared by a variety of synthetic methods known to those skilled in the art, including the specific Examples listed below, Examples formed by their combination with other chemical synthesis methods and equivalent alternatives well known to those skilled in the art. Preferred Examples include, but are not limited to, the Examples of the present invention. It will be apparent to those skilled in the art that various changes and improvements can be made to specific examples of the present invention without departing from the spirit and scope of the present invention.

### Example 1

### N-(2-(4,4-difluoropiperidin-1-yl)-6-methylpyrimidin-4-yl)-5-(2-hydroxyethylsulfonamido)-7-(6-azaspiro[2.5]octan-6-yl)imidazo[1,2-a]pyridine-8-carboxamide

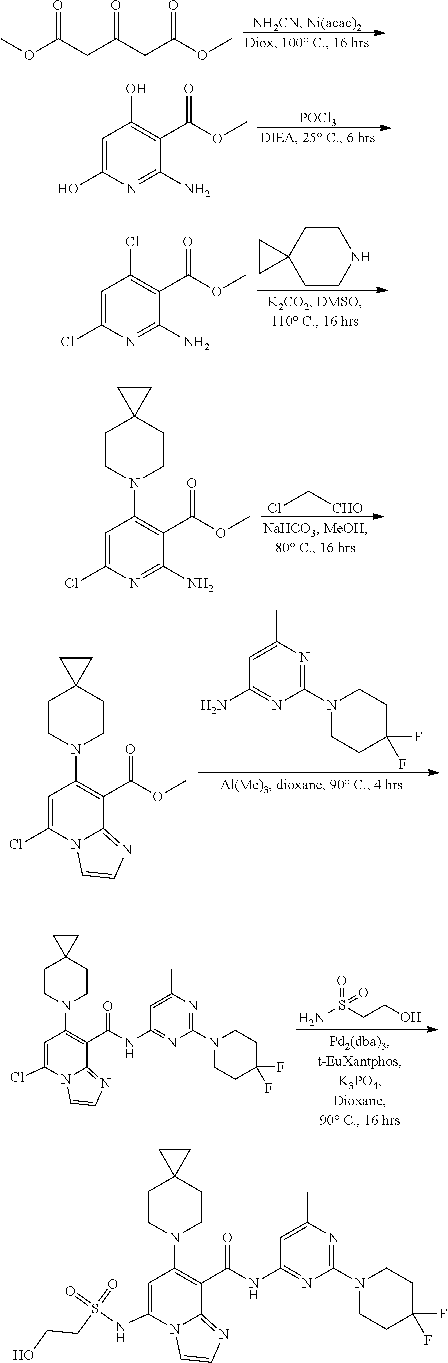

Step 1: To a solution of dimethyl 3-oxopentanedioate (20.0 g, 114 mmol) and aminonitrile (7.24 g, 172 mmol) in dioxane (100 mL) was added nickel acetylacetonate (2.95 g, 11.5 mmol). The mixture was heated to 100° C. and stirred for 16 h until the precipitation of a large amount of yellow solids. The precipitated solids were filtered and concentrated under reduced pressure to give methyl 2-amino-4,6-dihydroxynicotinate (7.50 g, 40.2 mmol, yield: 35%) as a yellow solid. LCMS (ESI): [M+H]⁺=184.9.

Step 2: To a solution of methyl 2-amino-4,6-dihydroxynicotinate (5.50 g, 29.9 mmol) in phosphorus oxychloride (50 mL) was added N,N-diisopropylethylamine (11.6 g, 89.6 mmol). The reaction mixture was stirred at 25° C. for 6 h. The reaction solution was concentrated under reduced pressure, and the reaction mixture was diluted with water (100 mL) and extracted with ethyl acetate (100 mL×3). The combined organic layer was dried over anhydrous sodium sulfate, and filtered, and the filtrate was concentrated under reduced pressure to give the residue. The residue was purified by flash column chromatography (silica gel, 0-15% gradient of ethyl acetate/petroleum ether) to give methyl 2-amino-4,6-dichloronicotinate (2.30 g, 10.4 mmol, yield: 35%) as a yellow solid. LCMS (ESI): [M+H]⁺=221.0.

Step 3: To a solution of methyl 2-amino-4,6-dichloronicotinate (2.30 g, 10.4 mmol) and 6-azaspiro[2.5]octane hydrochloride (1.54 g, 10.4 mmol) in DMSO (23 mL) was added potassium carbonate (3.74 g, 27.1 mmol). The mixture was heated to 110° C. and stirred for 16 h. The reaction solution was diluted with water (50 mL) and extracted with ethyl acetate (50 mL×3). The combined organic layer was dried over anhydrous sodium sulfate, and filtered, and the filtrate was concentrated under reduced pressure to give the residue. The residue was purified by flash column chromatography (silica gel, 0-15% gradient of ethyl acetate/petroleum ether) to give methyl 2-amino-6-chloro-4-(6-azaspiro[2.5]octan-6-yl)nicotinate (1.40 g, 4.68 mmol, yield: 45%) as a yellow solid. LCMS (ESI): [M+H]⁺=296.0. ¹H NMR (400 MHz, DMSO-d6) δ ppm 6.74-6.57 (m, 2H), 6.30-6.13 (m, 1H), 3.86-3.69 (m, 3H), 3.21-3.07 (m, 4H), 1.49-1.33 (m, 4H), 0.37-0.31 (m, 4H) Step 4: To a solution of methyl 2-amino-6-chloro-4-(6-azaspiro[2.5]octan-6-yl)nicotinate (1.40 g, 4.73 mmol) and sodium bicarbonate (0.80 g, 9.47 mmol) in methanol (12 mL) and water (6 mL) was added 2-chloroacetaldehyde (2.79 g, 14.2 mmol). The mixture was heated to 80° C. and stirred for 16 h. The reaction solution was diluted with water (20 mL) and extracted with ethyl acetate (20 mL×3). The combined organic layer was dried over anhydrous sodium sulfate, and filtered, and the filtrate was concentrated under reduced pressure to give the residue. The residue was purified by flash column chromatography (silica gel, 0-20% gradient of ethyl acetate/petroleum ether) to give methyl 5-chloro-7-(6-azaspiro[2.5]octan-6-yl)imidazo[1,2-a]pyridine-8-carboxylate (1.20 g, 3.40 mmol, yield: 72%) as a yellow solid. LCMS (ESI): [M+H]⁺=319.9.

Step 5: To a solution of methyl 5-chloro-7-(6-azaspiro[2.5]octan-6-yl)imidazo[1,2-a]pyridine-8-carboxylate (1.00 g, 3.13 mmol) in THE (20 mL) was slowly added a solution of trimethylaluminum (4.69 mL, 2 M, 9.38 mmol) in toluene dropwise at 5-10° C. The mixture was stirred at 15° C. for 0.5 h, and 2-(4,4-difluoropiperidin-1-yl)-6-methylpyrimidin-4-amine (1.07 g, 4.69 mmol) was added to the reaction solution. The mixture was heated to 90° C. and stirred for 4 h. The reaction was cooled to 25° C., quenched with ice water (20 mL) and extracted with ethyl acetate (20 mL×3). The combined organic layer was dried over anhydrous sodium sulfate, and filtered, and the filtrate was concentrated under reduced pressure to give the residue. The residue was purified by flash column chromatography (silica gel, 0-10% gradient of ethyl acetate/petroleum ether) to give 5-chloro-N-(2-(4,4-difluoropiperidin-1-yl)-6-methylpyrimidin-4-yl)-7-(6-azaspiro[2.5]octan-6-yl)imidazo[1,2-a]pyridine-8-carboxamide (0.30 g, 0.59 mmol, yield: 19%) as a yellow solid. LCMS (ESI): [M+H]⁺=516.1.

Step 6: To a solution of 5-chloro-N-(2-(4,4-difluoropiperidin-1-yl)-6-methylpyrimidin-4-yl)-7-(6-azaspiro[2.5]octan-6-yl)imidazo[1,2-a]pyridine-8-carboxamide (300 mg, 0.58 mmol), 2-hydroxyethanesulfonamide (94.6 mg, 0.76 mmol), potassium phosphate (500 mg, 2.33 mmol) and 2-di-tert-butylphosphine-2′,4′,6′-triisopropylbiphenyl (49.4 mg, 0.12 mmol) in dioxane (10 mL) was added tris(dibenzylideneacetone)dipalladium (54.3 mg, 0.06 mmol). The mixture was heated to 90° C. and stirred for 16 h under nitrogen atmosphere. The reaction solution was diluted with water (10 mL) and extracted with ethyl acetate (10 mL×3). The combined organic layer was dried over anhydrous sodium sulfate, and filtered, and the filtrate was concentrated under reduced pressure to give the residue. The residue was purified by preparative HPLC (C18, 20-60% gradient of acetonitrile/water) to give N-(2-(4,4-difluoropiperidin-1-yl)-6-methylpyrimidin-4-yl)-5-(2-hydroxyethylsulfonamido)-7-(6-azaspiro[2.5]octan-6-yl)imidazo[1,2-a]pyridine-8-carboxamide (122 mg, 0.04 mmol, yield: 35%) as a yellow solid compound. LCMS (ESI): [M+H]⁺=605.1. ¹H NMR (400 MHz, DMSO-d₆) δ ppm 13.08-12.87 (m, 1H), 12.83-12.58 (m, 1H), 7.93-7.79 (m, 1H), 7.71-7.63 (m, 1H), 7.49-7.41 (m, 1H), 6.87-6.78 (m, 1H), 3.99-3.88 (m, 4H), 3.85-3.77 (m, 2H), 3.28-3.20 (m, 2H), 3.11-3.01 (m, 4H), 2.39-2.32 (m, 3H), 2.11-1.96 (m, 4H), 1.87-1.61 (m, 4H), 0.45-0.33 (m, 4H).

### Example 2

### N-(2-(4,4-difluoropiperidin-1-yl)-6-methylpyrimidin-4-yl)-4-(2-hydroxyethylsulfonamido)-6-(6-azaspiro[2.5]octan-6-yl)-benzofuran-7-carboxamide

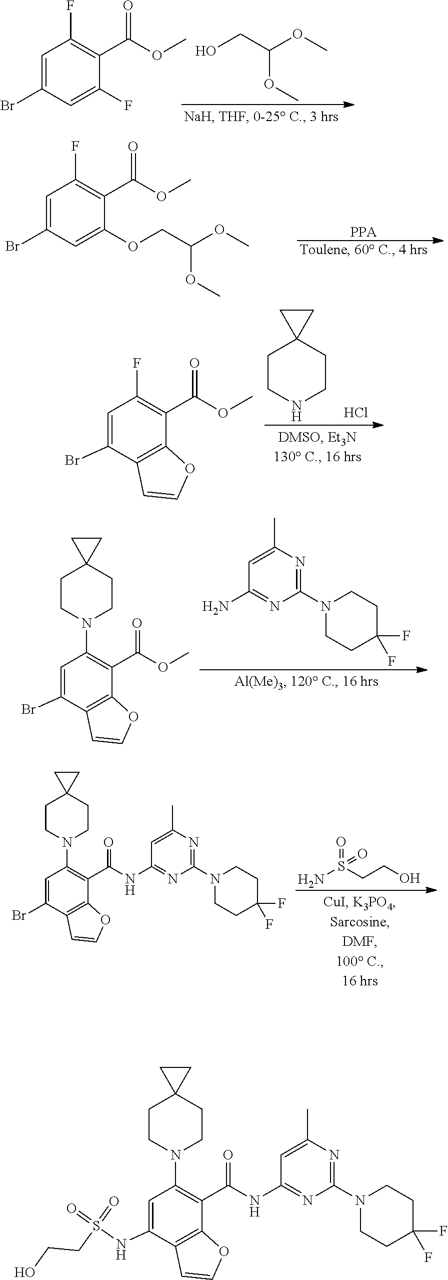

Step 1: To a solution of 2,2-dimethoxyethanol (8.88 g, 83.6 mmol) in THF (400 mL) was slowly added sodium hydride (3.51 g, 87.6 mmol, 60% purity) at 0° C. The mixture was stirred at 25° C. for 1 h. Then, methyl 4-bromo-2,6-difluorobenzoate (20.0 g, 79.6 mmol) was added to the mixture. The mixture was stirred at 25° C. for 2 h. The reaction mixture was diluted with water (200 mL) and extracted with ethyl acetate (200 mL×2). The combined organic layer was dried over anhydrous sodium sulfate and filtered. The filtrate was concentrated under reduced pressure to give a residue. The residue was purified by flash column chromatography (silica gel, 0-10% gradient of ethyl acetate/petroleum ether) to give methyl 4-bromo-2-(2,2-dimethoxyethoxy)-6-fluorobenzoate (17.0 g, 50.2 mmol, yield: 63%) as a yellow solid. ¹H NMR (400 MHz, DMSO-d₆) δ ppm 7.34-7.27 (m, 2H), 4.61 (dd, J=4.8 5.6 Hz, 1H), 4.11 (d, J=5.2 Hz, 2H), 3.82 (s, 3H), 3.34 (s, 6H)

Step 2: The solution of polyphosphoric acid (52.0 g, 113 mmol) in toluene (400 mL) was stirred at 60° C. for 30 min. Then, methyl 4-bromo-2-(2,2-dimethoxyethoxy)-6-fluorobenzoate (19.0 g, 56.4 mmol) was added to the mixture. The mixture was stirred at 60° C. for 4 h. The reaction mixture was diluted with water (200 mL) and extracted with ethyl acetate (200 mL×2). The combined organic layer was dried over anhydrous sodium sulfate, and filtered, and the filtrate was concentrated under reduced pressure to give the residue. The residue was purified by flash column chromatography (silica gel, 0-10% gradient of ethyl acetate/petroleum ether) to give methyl 4-bromo-6-fluorobenzofuran-7-carboxylate (9.70 g, 35.5 mmol, yield: 63%) as a yellow solid. LCMS (ESI): [M+H]⁺=274.8. ¹H-NMR (400 MHz, DMSO-d₆) δ ppm 8.26 (d, J=1.0 Hz, 1H), 7.70 (d, J=10.8 Hz, 1H), 7.04 (d, J=1.0 Hz, 1H), 3.93 (s, 3H)

Step 3: To a solution of methyl 4-bromo-6-fluorobenzofuran-7-carboxylate (3.00 g, 11.0 mmol) and triethylamine (3.33 g, 32.9 mmol) in DMSO (30 mL) was added 6-azaspiro[2.5]octane hydrochloride (1.78 g, 12.1 mmol). The mixture was stirred at 130° C. for 16 h. The reaction solution was diluted with water (30 mL) and extracted with ethyl acetate (30 mL×3). The combined organic layer was dried over anhydrous sodium sulfate, and filtered, and the filtrate was concentrated under reduced pressure to give the residue. The residue was purified by flash column chromatography (silica gel, 0-8% gradient of THF/petroleum ether) to give methyl 4-bromo-6-(6-azaspiro[2.5]octan-6-yl)benzofuran-7-carboxylate (1.20 g, 0.30 mmol, yield: 30%) as a yellow solid. LCMS (ESI): [M+H]⁺=363.8. ¹H-NMR (400 MHz, DMSO-d₆) δ ppm 8.04 (d, J=2.4 Hz, 1H), 7.27 (s, 1H), 6.87 (d, J=2.0 Hz, 1H), 3.89 (s, 3H), 2.94-2.87 (m, 4H), 1.35-1.27 (m, 4H), 0.31 (s, 4H) Step 4: To a solution of 2-(4,4-difluoropiperidin-1-yl)-6-methylpyrimidin-4-amine (1.13 g, 4.94 mmol) in trimethylaluminum (9.90 mL, 2M) in toluene was added methyl 4-bromo-6-(6-azaspiro[2.5]octan-6-yl)benzofuran-7-carboxylate (1.20 g, 3.29 mmol) at 0° C. The mixture was heated to 120° C. and stirred for 16 h. The mixture was cooled to ambient temperature, slowly poured into ice water (30 mL) and extracted with ethyl acetate (30 mL×3). The combined organic layer was dried over anhydrous sodium sulfate, and filtered, and the filtrate was concentrated under reduced pressure to give the residue. The residue was purified by flash column chromatography (silica gel, 0-8% gradient of THF/petroleum ether) to give 4-bromo-N-(2-(4,4-difluoropiperidin-1-yl)-6-methylpyrimidin-4-yl)-6-(6-azaspiro[2.5]octan-6-yl)benzofuran-7-carboxamide (0.65 g, 1.15 mmol, yield: 35%) as a yellow solid. LCMS (ESI): [M+H]⁺=562.0.

Step 5: To a solution of 2-hydroxyethanesulfonamide (174 mg, 1.39 mmol), 2-(methylamino)acetic acid (95.4 mg, 1.07 mmol), and potassium phosphate (927 mg, 4.28 mmol) in N,N-dimethylformamide (12 mL) was added cuprous iodide (104 mg, 0.54 mmol). The mixture was stirred at 50° C. for 5 min. Then, 4-bromo-N-(2-(4,4-difluoropiperidin-1-yl)-6-methylpyrimidin-4-yl)-6-(6-azaspiro[2.5]octan-6-yl)benzofuran-7-carboxamide (600 mg, 1.07 mmol) was added to the reaction mixture at 50° C. The mixture was heated to 100° C. and stirred for 16 h under nitrogen atmosphere. Water (10 mL) was added to the reaction solution and extracted with ethyl acetate (10 mL×2). The combined organic layer was dried over anhydrous sodium sulfate, and filtered, and the filtrate was concentrated under reduced pressure to give the residue. The residue was purified by preparative HPLC (C18, 38-78% gradient of water (formic acid)/acetonitrile) to give N-(2-(4,4-difluoropiperidin-1-yl)-6-methylpyrimidin-4-yl)-4-(2-hydroxyethylsulfonamido)-6-(6-azaspiro[2.5]octan-6-yl)benzofuran-7-carboxamide (260 mg, 0.40 mmol, yield: 38%) as a white solid compound. LCMS (ESI): [M+H]⁺=605.5. ¹H-NMR (400 MHz, DMSO-d₆) δ ppm 12.87 (s, 1H), 10.31 (s, 1H), 8.01 (d, J=2.0 Hz, 1H), 7.51-7.23 (m, 3H), 4.94 (m, 1H), 4.09-3.81 (m, 4H), 3.81-3.70 (m, 2H), 3.44-3.36 (m, 2H), 3.09-2.94 (m, 4H), 2.32 (s, 3H), 2.08-1.90 (m, 4H), 1.79-1.57 (m, 4H), 0.38 (s, 4H)

### Example 3

### N-(2-(4,4-difluoropiperidin-1-yl)-6-methylpyrimidin-4-yl)-4-(2-hydroxyethylsulfonamido)-6-(6-azaspiro[2.5]octan-6-yl)-2,3-dihydrobenzofuran-7-carboxamide

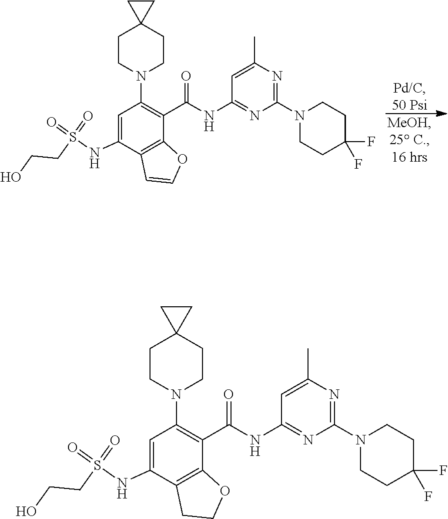

Step 1: To a solution of N-(2-(4,4-difluoropiperidin-1-yl)-6-methylpyrimidin-4-yl)-4-(2-hydroxyethylsulfonamido)-6-(6-azaspiro[2.5]octan-6-yl)benzofuran-7-carboxamide (100 mg, 0.17 mmol) in methanol (2 mL) was added palladium/carbon (20 mg, 10% purity). The mixture was stirred at 25° C. for 16 h under hydrogen atmosphere (50 Psi). The mixture was filtered by diatomite. The filtrate was concentrated under reduced pressure. The residue was purified by preparative HPLC (C18, 24-64% gradient of water (formic acid)/acetonitrile) to give N-(2-(4,4-difluoropiperidin-1-yl)-6-methylpyrimidin-4-yl)-4-(2-hydroxyethylsulfonamido)-6-(6-azaspiro[2.5]octan-6-yl)-2,3-dihydrobenzofuran-7-carboxamide (40.0 mg, 0.07 mmol, yield: 40%) as a white solid compound. LCMS (ESI): [M+H]⁺=607.5. ¹H-NMR (400 MHz, DMSO-d₆) δ ppm 11.88 (s, 1H), 9.58 (s, 1H), 7.31 (s, 1H), 6.82 (s, 1H), 5.01 (br s, 1H), 4.56 (t, J=1.0 Hz, 2H), 3.99-3.81 (m, 4H), 3.76 (t, J=1.0 Hz, 2H), 3.32-3.30 (m, 2H), 3.13 (t, J=1.0 Hz, 2H), 2.98-2.84 (m, 4H), 2.26 (s, 3H), 2.03-1.91 (m, 4H), 1.64-1.45 (m, 4H), 0.32 (s, 4H)

### Example 4

### N-(2-(4,4-difluoropiperidin-1-yl)-6-methylpyrimidin-4-yl)-4-(2-_hydroxyethylsulfonamido)-2-(6-azaspiro[2.5]octan-6-yl)-1-naphthamide

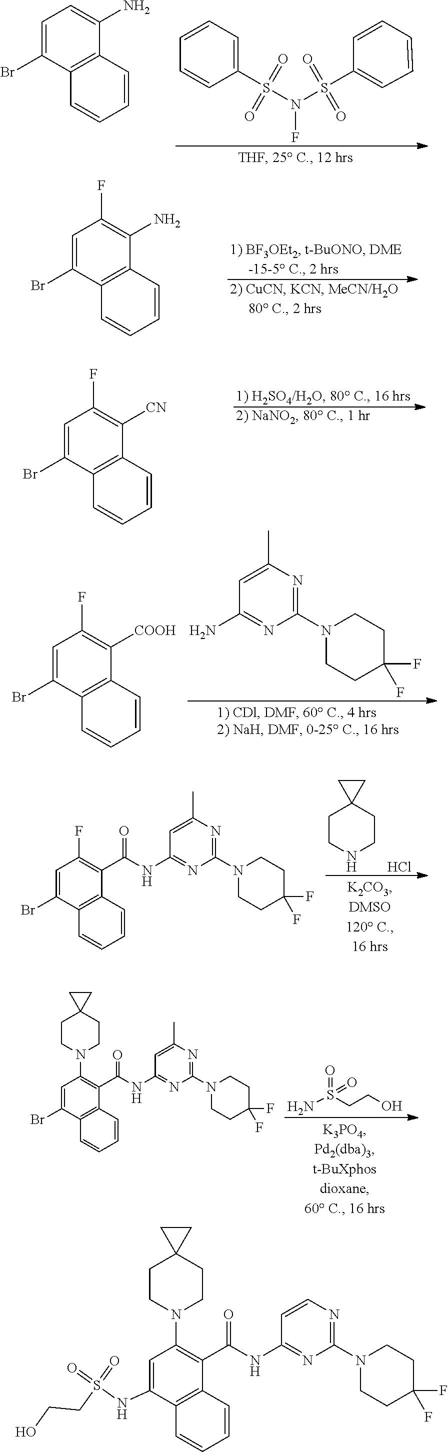

Step 1 4-bromonaphthalene-1-amine (30.0 g, 135 mmol) was dissolved in THF (300 mL) and N-fluoro-N-(phenylsulfonyl)benzenesulfonamide (51.1 g, 162 mmol) was added. The mixture was stirred at 25° C. for 12 h. The reaction solution was diluted with water (500 mL) and extracted with ethyl acetate (300 mL×3). The organic layer was dried over anhydrous sodium sulfate, filtered and concentrated. The residue was purified by flash column chromatography (silica gel, 0-5% gradient of ethyl acetate/petroleum ether) to give 4-bromo-2-fluoronaphthalen-1-amine (12.8 g, 52.7 mmol, yield 39%) as a brown solid, LCMS (ESI): [M+H]⁺=239.9.

Step 2: Boron trifluoride diethyl etherate (8.87 g, 62.5 mmol) was cooled to −15° C. in a three-necked flask. 4-bromo-2-fluoronaphthalen-1-amine (10.0 g, 41.6 mmol) in the solvent of N,N-dimethylacetamide (100 mL) and tert-butyl nitrite (5.70 g, 54.1 mmol) in the solvent of N,N-dimethylacetamide (100 mL) were added dropwise to the above three-necked flask. and the solution was stirred at −15° C. for 1 h. The temperature was raised to 5° C., and n-pentane (200 mL) was added. The solution was filtered and washed with n-pentane (200 mL) to give a brown solid. The filtered brown solid was dissolved in acetonitrile (180 mL). Potassium cyanide (7.52 g, 115.47 mmol) and cuprous cyanide (4.00 g, 43.7 mmol) were dissolved in acetonitrile (90 mL) and pure water (30 mL). The brown solid dissolved in acetonitrile was added to the reaction solution. The solution was heated to 80° C. and stirred for 2 h. The reaction solution was quenched with saturated sodium carbonate solution (500 mL) and extracted with ethyl acetate (400 mL×2). The organic layer was dried, filtered and concentrated. The residue was purified by flash column chromatography (silica gel, 0-5% gradient of THF/petroleum ether) to give 4-bromo-2-fluoro-1-naphthonitrile (4.90 g, 14.99 mmol, yield: 36%) as a brown solid. ¹H NMR (400 MHz, DMSO-d₆) δ ppm 8.33-8.25 (m, 2H), 8.15-8.08 (m, 1H), 7.96-7.84 (m, 2H) Step 3: To a solution of sulfuric acid (100 mL, 70%) was added 4-bromo-2-fluoro-1-naphthonitrile (4.90 g, 19.6 mmol). The solution was stirred at 80° C. for 16 h. Sodium nitrite (1.94 g, 28.1 mmol) was added at 80° C. The solution was stirred at 80° C. for 1 h. The reaction solution was diluted with water (50 mL) and extracted with ethyl acetate (100 mL×3). The organic layer was dried, filtered and concentrated. The residue was slurried with dichloromethane (80 mL) at 25° C. for 16 h to give 4-bromo-2-fluoro-1-naphthoic acid (4.00 g, 14.9 mmol, yield: 76%) as a brown solid. ¹H NMR (400 MHz, DMSO-d₆) δ ppm 8.25-8.19 (m, 1H), 8.07 (d, J=9.2 Hz, 2H), 7.79-7.72 (m, 2H) Step 4: 4-bromo-2-fluoro-1-naphthoic acid (2.00 g, 7.43 mmol) and N,N carbonyldiimidazole (7.23 g, 44.6 mmol) were dissolved in N,N-dimethylformamide (40.0 mL). The mixture was heated to 60° C. and stirred for 4 h. 2-(4,4-difluoropiperidin-1-yl)-6-methylpyrimidin-4-amine (2.04 g, 8.92 mmol) was dissolved in N,N-dimethylformamide (40 mL), then added with sodium hydrogen (0.89 g, 22.3 mmol, 60% mass fraction) at 0° C., and stirred at 25° C. for 30 min. The above solution was added to the reaction system and stirred at 25° C. for 16 h. The reaction solution was quenched with water (200 mL) and extracted with ethyl acetate (200 mL×2). The organic layer was dried, filtered and concentrated to give a residue. The residue was purified by flash column chromatography (silica gel, 0-7% gradient of THF/petroleum ether) to give 4-bromo-N-(2-(4,4-difluoropiperidin-1-yl)-6-methylpyrimidin-4-yl)-2-fluoro-1-naphthamide (1.10 g, 2.19 mmol, yield: 20%) as a yellow solid. LCMS (ESI): [M+H]⁺=480.9, ¹H NMR (400 MHz, DMSO-d₆) δ ppm 11.29 (br s, 1H), 8.26-8.21 (m, 1H), 8.10 (d, J=8.8 Hz, 1H), 7.87 (br d, J=2.0 Hz, 1H), 7.76-7.71 (m, 2H), 7.47 (br s, 1H), 3.86 (br s, 4H), 2.36 (br s, 3H), 1.97 (br s, 4H)

Step 5:4-Bromo-N-(2-(4,4-difluoropiperidin-1-yl)-6-methylpyrimidin-4-yl)-2-fluoro-1-naphthamide (1.10 g, 93%) and 6-azaspiro[2.5]octane hydrochloride (0.44 g, 2.98 mmol) were dissolved in DMSO (40.0 mL), then potassium carbonate (0.95 g, 6.89 mmol) was added, and reaction solution was stirred at 120° C. for 16 h. The reaction solution was diluted with water (100 mL) and extracted with ethyl acetate (100 mL×2). The organic layer was dried, filtered and concentrated. The residue was purified by flash column chromatography (silica gel, 0-7% gradient of THF/petroleum ether) to give 4-bromo-N-(2-(4,4-difluoropiperidin-1-yl)-6-methylpyrimidin-4-yl)-2-(6-azaspiro[2.5]octan-6-yl)-1-naphthamide (0.36 g, 0.71 mmol, yield: 31%) as a yellow solid. LCMS (ESI): [M+H]⁺=572.2, ¹H NMR (400 MHz, DMSO-d₆) δ ppm 10.75 (br s, 1H), 8.18-8.08 (m, 1H), 7.91-7.78 (m, 2H), 7.63-7.57 (m, 2H), 7.49 (br s, 1H), 3.87 (br s, 4H), 3.11 (br s, 4H), 2.36 (br s, 3H), 2.12-1.94 (m, 4H), 1.42-1.35 (m, 4H), 0.28 (s, 4H)

Step 6: 4-Bromo-N-(2-(4,4-difluoropiperidin-1-yl)-6-methylpyrimidin-4-yl)-2-(6-azaspiro[2.5]octan-6-yl)-1-naphthamide (200 mg, 0.35 mmol) and 2-hydroxyethanesulfonamide (57.0 mg, 0.46 mmol) were dissolved in dioxane (10.0 mL), and added with potassium phosphate (297 mg, 1.40 mmol), 2-di-tert-butylphosphine-2′,4′,6′-triisopropylbiphenyl (29.8 mg, 0.07 mmol) and tris(dibenzylideneacetone)dipalladium (32.1 mg, 0.04 mmol). The solution was stirred at 60° C. for 16 h under nitrogen atmosphere The reaction solution was filtered, diluted, and purified by preparative HPLC (C18, 28-68% gradient water (ammonia+ammonium bicarbonate)/acetonitrile) to give N-(2-(4,4-difluoropiperidin-1-yl)-6-methylpyrimidin-4-yl)-4-(2-hydroxyethylsulfonamido)-2-(6-azaspiro[2.5]octan-6-yl)-1-naphthamide (27.7 mg, 0.05 mmol, yield: 13%) as a white solid. LCMS (ESI): [M+H]⁺=615.2, ¹H NMR (400 MHz, DMSO-d₆) δ ppm 10.74 (br s, 1H), 9.90 (br s, 1H), 8.25 (d, J=8.4 Hz, 1H), 7.90 (br d, J=7.6 Hz, 1H), 7.62-7.37 (m, 4H), 5.02 (s, 1H), 3.97-3.71 (m, 6H), 3.32-3.28 (m, 2H), 3.09 (br s, 4H), 2.40-2.29 (m, 3H), 2.02-1.90 (m, 4H), 1.44-1.36 (m, 4H), 0.29 (s, 4H)

### Example 5

### N-(2-(4,4-difluoropiperidin-1-yl)-6-methylpyrimidin-4-yl)-8-(2-hydroxyethanesulfonamido)-6-(6-azaspiro[2.5]octan-6-yl)quinoline-5-carboxamide

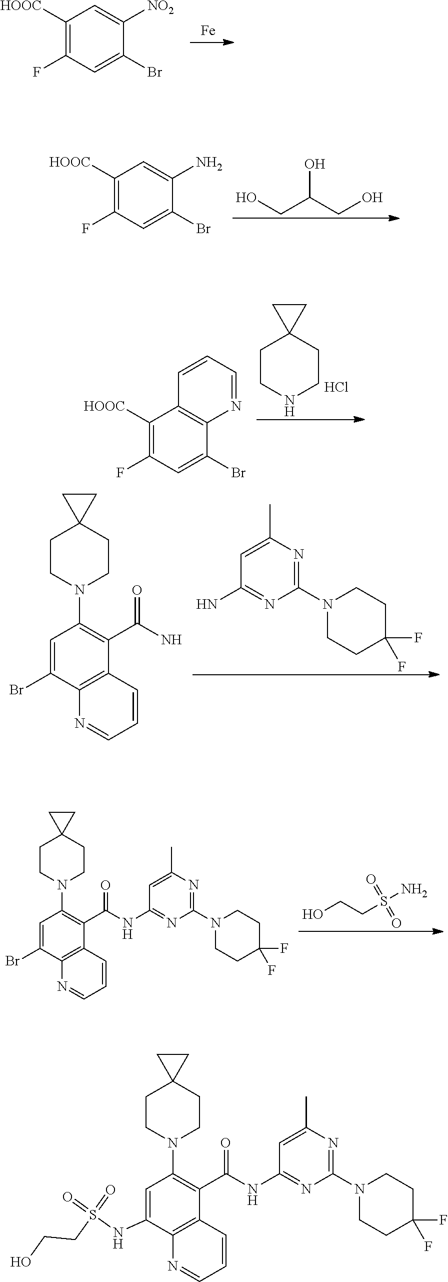

Step 1: To a mixed solution of ethanol (40 mL), water (10 mL) and THE (40 mL) were added iron powder (8.55 g, 151.51 mmol), 4-bromo-2-fluoro-5-nitrobenzoic acid (8.00 g, 30.30 mmol) and ammonium chloride (2.45 g, 45.45 mmol). The mixture was heated to 90° C. and stirred for 3 h. The mixture was cooled to 40° C., and the hot reaction solution was filtered through a diatomite sand core funnel. The filtrate was concentrated to give 5-amino-4-bromo-2-fluorobenzoic acid (6.00 g, 25.63 mmol, yield: 84.6%) as a yellow solid. LCMS (ESI): [M+H]⁺=234.1

Step 2: Glycerol (5.96 g, 64.10 mmol) was added to a mixed solution of concentrated sulfuric acid (20 mL) and water (5 mL), and the mixture was heated to 100° C. and stirred for 15 min. The mixture was cooled to 40° C., 5-amino-4-bromo-2-fluorobenzoic acid (5.00 g, 21.36 mmol) and sodium 3-nitrobenzenesulfonate (4.85 g, 21.36 mmol) were added thereto, and the mixture was heated to 100° C. and stirred for 15 h. After completion of the reaction, the reaction mixture was diluted with water (100 mL) and extracted with ethyl acetate (100 mL×3). The combined organic layer was dried over anhydrous sodium sulfate, and filtered, and the filtrate was concentrated under reduced pressure to give the residue. The residue was purified by flash column chromatography (silica gel, 0-20% gradient of methanol/dichloromethane) to give 8-bromo-6-fluoroquinoline-5-carboxylic acid (3.30 g, 12.21 mmol, yield: 57.3%) as a yellow solid. LCMS (ESI): [M+H]⁺=270.0; ¹HNMR (400 MHz, DMSO) δ 9.06 (dd, J=4.1, 1.6 Hz, 1H), 8.60 (dd, J=8.6, 1.6 Hz, 1H), 8.33 (d, J=9.4 Hz, 1H), 7.77 (dd, J=8.6, 4.1 Hz, 1H).

Step 3: To a solution of 8-bromo-6-fluoroquinoline-5-carboxylic acid (2.00 g, 7.41 mmol) in DMSO (20 mL) were added 6-azaspiro[2.5]octane hydrochloride (1.66 g, 11.10 mmol) and potassium carbonate (3.13 g, 22.2 mmol). The mixture was heated to 120° C. and stirred for 36 h. At the end of the reaction, the aqueous layer pH was adjusted to 6 with saturated aqueous sodium bicarbonate (20 mL) and extracted with ethyl acetate (50 mL×3). The combined organic layer was washed with saturated aqueous sodium chloride solution (50 mL), dried over anhydrous sodium sulfate and concentrated under reduced pressure. The crude was purified by flash column chromatography (silica gel, 0-20% gradient of methanol/dichloromethane) to give 8-bromo-6-(6-azaspiro[2.5]octan-6-yl)quinoline-5-carboxylic acid (1.50 g, 4.15 mmol, yield: 56.2%) as a yellow solid. LCMS (ESI): [M+H]⁺=361.2

Step 4: To a solution of 2-(4,4-difluoropiperidin-1-yl)-6-methylpyrimidin-4-amine (758 mg, 3.32 mmol) and 8-bromo-6-(6-azaspiro[2.5]octan-6-yl)quinoline-5-carboxylic acid (1.00 g, 1.30 mmol) in dichloromethane (10.0 mL) were added N,N,N′,N′-tetramethyl-O-(7-azabenzotriazol-1-yl)uronium hexafluorophosphate (1.61 g, 4.15 mmol) and potassium tert-butoxide (941 mg, 8.30 mmol). The mixture was stirred at 100° C. for 24 h. After completion of the reaction, the reaction mixture was diluted with water (10 mL) and extracted with dichloromethane (20 mL×3). The combined organic layer was dried over anhydrous sodium sulfate, and filtered, and the filtrate was concentrated under reduced pressure to give the residue. The residue was purified by flash column chromatography (silica gel, 0-50% gradient of ethyl acetate/petroleum ether) to give 8-bromo-N-(2-(4,4-difluoropiperidin-1-yl)-6-methylpyrimidin-4-yl)-6-(6-azaspiro[2.5]octan-6-yl)quinoline-5-carboxamide (200 mg, 0.35 mmol, yield: 12.6%) as a yellow solid. LCMS (ESI): [M+H]⁺=571.3

Step 5: 2-hydroxyethanesulfonamide (67 mg, 1.40 mmol), tris(dibenzylideneacetone)dipalladium (33 mg, 0.03 mmol), potassium phosphate (303 mg, 1.40 mmol), and 2-di-tert-butylphosphine-2′,4′,6′-triisopropylbiphenyl (30 mg, 0.06 mmol) were placed in a flask under nitrogen atmosphere, and 1,4-dioxane (4 mL) was added to the reaction flask. The mixture was stirred at 50° C. for 0.5 h and then added with 8-bromo-N-(2-(4,4-difluoropiperidin-1-yl)-6-methylpyrimidin-4-yl)-6-(6-azaspiro[2.5]octan-6-yl)quinoline-5-carboxamide (200 mg, 0.35 mmol). The temperature of the reaction system was raised to 120° C. and the system was stirred for 5 h. At the end of reaction, the system was cooled to ambient temperature, then poured into water (10 mL) and extracted with dichloromethane (30 mL×3). The organic layer was dried with anhydrous sodium sulfate, filtered, concentrated and purified by preparative HPLC (C18, 0-100% gradient of acetonitrile/water) to give N-(2-(4,4-difluoropiperidin-1-yl)-6-methylpyrimidin-4-yl)-8-(2-hydroxyethanesulfonamido)-6-(6-azaspiro[2.5]octan-6-yl)quinoline-5-carboxamide (20 mg, 0.032 mmol, yield: 9.3%) as a yellow solid compound. LCMS(ESI) m/z: [M+H]⁺=616.4; 1H NMR (400 MHz, DMSO) δ 10.98 (s, 1H), 10.06-9.08 (m, 1H), 8.80 (d, J=3.9 Hz, 1H), 8.60 (s, 1H), 8.00-7.39 (m, 3H), 5.14 (s, 1H), 4.02-3.75 (m, 6H), 3.50 (q, J=4.9 Hz, 2H), 3.16 (s, 4H), 2.37 (s, 3H), 1.96 (d, J=20.8 Hz, 4H), 1.49 (s, 4H), 0.33 (d, J=2.7 Hz, 4H).

### Example 6

### 2-{6-Azaspiro[2.5]octan-6-yl}-N-[8-(4,4-difluoropiperidin-1-yl)-7-fluoroquinolin-6-yl]-4-(2-hydroxyethanesulfonamido)naphthalene-1-carboxamide

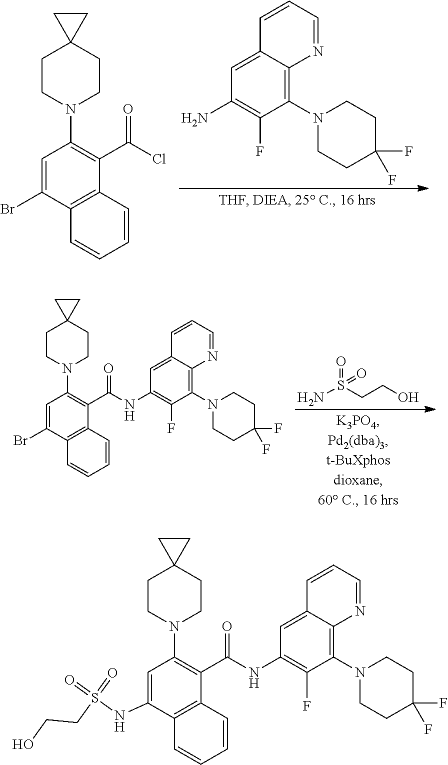

Step 1: To a solution of 8-(4,4-difluoropiperidin-1-yl)-7-fluoroquinolin-6-amine (171 mg, 0.61 mmol) and 2-{6-azaspiro[2.5]octan-6-yl}-4-bromonaphthalene-1-carbonyl chloride (230 mg, 0.61 mmol) in THF (6 mL) was added N,N-diisopropylethylamine (240 mg, 1.82 mmol). The mixture was stirred at 25° C. for 16 h. The reaction solution was diluted with water (20 mL) and extracted with ethyl acetate (20 mL×3). The combined organic layer was dried over anhydrous sodium sulfate, and filtered, and the filtrate was concentrated under reduced pressure to give the residue. The residue was purified by flash column chromatography (silica gel, 0-10% gradient of THF/petroleum ether) to give 2-{6-azaspiro[2.5]octan-6-yl}-4-bromo-N-[8-(4,4-difluoropiperidin-1-yl)-7-fluoroquinolin-6-yl]naphthalene-1-carboxamide (250 mg, 0.40 mmol, yield: 66%) as a yellow solid. LCMS (ESI): [M+H]⁺=625.1; ¹H NMR (400 MHz, DMSO-d₆) δ ppm 0.45 (s, 1H), 8.88 (dd, J=1.2, 4.4 Hz, 1H), 8.47-8.36 (m, 2H), 8.23-8.11 (m, 1H), 7.97 (d, J=8.0 Hz, 1H), 7.88 (s, 1H), 7.70-7.58 (m, 2H), 7.53 (dd, J=4.0, 8.4 Hz, 1H), 3.56 (br t, J=4.8 Hz, 4H), 3.20-3.15 (m, 4H), 2.26-2.12 (m, 4H), 1.49-1.39 (m, 4H), 0.29 (s, 4H)

Step 2: 2-{6-azaspiro[2.5]octan-6-yl}-4-bromo-N-[8-(4,4-difluoropiperidin-1-yl)-7-fluoroquinolin-6-yl]naphthalene-1-carboxamide (210 mg, 0.33 mmol) and 2-hydroxyethanesulfonamide (84.3 mg, 0.67 mmol) were dissolved in dioxane (4 mL), and added with potassium phosphate (214 mg, 1.01 mmol), 2-di-tert-butylphosphine-2′,4′,6′-triisopropylbiphenyl (14.3 mg, 0.03 mmol) and tris(dibenzylideneacetone)dipalladium (31.4 mg, 0.03 mmol). The solution was stirred at 60° C. for 16 h under nitrogen atmosphere. The reaction solution was filtered, diluted, and purified by preparative HPLC (C18, 34-74% gradient of water (formic acid)/acetonitrile) to give 2-{6-azaspiro[2.5]octan-6-yl}-N-[8-(4,4-difluoropiperidin-1-yl)-7-fluoroquinolin-6-yl]-4-(2-hydroxyethanesulfonamido)naphthalene-1-carboxamide (56 mg, 0.08 mmol, yield: 25%) as a yellow solid. LCMS (ESI): [M+H]⁺=668.3; ¹H NMR (400 MHz, DMSO-d₆) δ ppm 10.45 (s, 1H), 10.16-9.47 (m, 1H), 8.87 (dd, J=1.6, 4.0 Hz, 1H), 8.42 (d, J=8.0 Hz, 2H), 8.28 (d, J=8.4 Hz, 1H), 7.99 (d, J=8.4 Hz, 1H), 7.62-7.45 (m, 4H), 5.31-4.83 (m, 1H), 3.83 (t, J=6.4 Hz, 2H), 3.55 (br d, J=4.8 Hz, 4H), 3.32-3.30 (m, 2H), 3.18-3.11 (m, 4H), 2.26-2.12 (m, 4H), 1.43 (br s, 4H), 0.30 (s, 4H)

### Example 7

### 2-{6-Azaspiro[2.5]octan-6-yl}-N-[2-(4,4-difluoropiperidin-1-yl)-6-methoxypyrimidin-4-yl]-4-(2-hydroxyethanesulfonamido)naphthalene-1-carboxamide

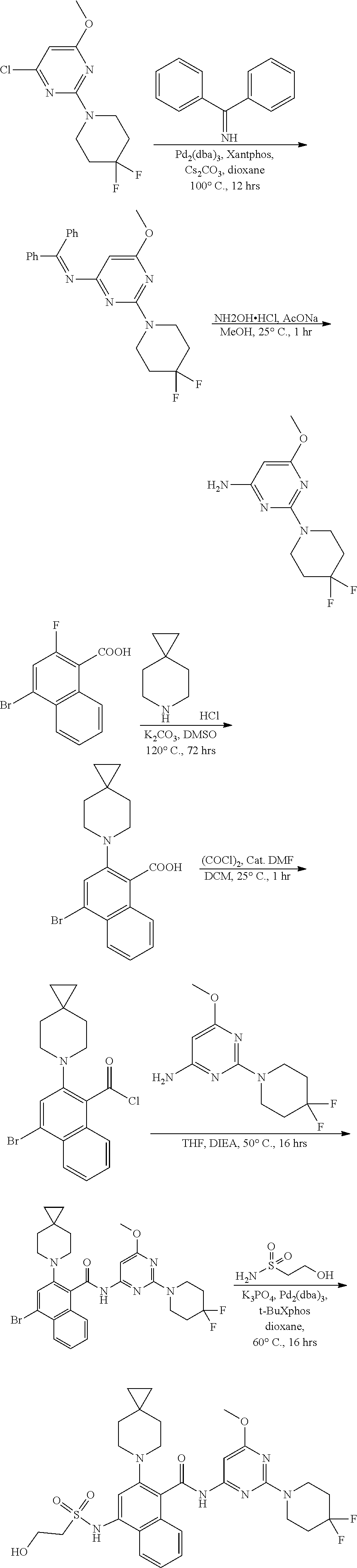

Step 1: To a solution of 4-chloro-2-(4,4-difluoropiperidin-1-yl)-6-methoxypyrimidine (5.00 g, 18.9 mmol) in dioxane (100 mL) were added benzophenone imine (5.16 g, 28.5 mmol), cesium carbonate (18.9 g, 56.9 mmol), 4,5-bis(diphenylphosphino)-9,9-dimethylxanthene (1.12 g, 1.90 mmol) and tris(dibenzylideneacetone)dipalladium (1.77 g, 1.90 mmol). The reaction solution was replaced with nitrogen three times, heated to 100° C. under nitrogen atmosphere and stirred for 12 h. The reaction solution was cooled to ambient temperature and extracted with water (200 mL) and ethyl acetate (200 mL×3). The combined organic layer was washed with saturated saline (200 mL), dried over anhydrous sodium sulfate and filtered. The filtrate was concentrated under reduced pressure to give a residue. The residue was slurried with ethyl acetate to give N-[2-(4,4-difluoropiperidin-1-yl)-6-methoxypyrimidin-4-yl]-1,1-diphenylmethanimine (5.90 g, 14.4 mmol, yield: 76%) as a yellow solid. LCMS (ESI): [M+H]+=409.2; ¹H NMR (400 MHz, DMSO-d₆) δ ppm 7.81-7.13 (m, 10H), 5.50 (s, 1H), 3.77-3.70 (m, 7H), 1.95-1.75 (m, 4H) Step 2: To a solution of N-[2-(4,4-difluoropiperidin-1-yl)-6-methoxypyrimidin-4-yl]-1,1-diphenylmethanimine (1.00 g, 2.45 mmol) in methanol (10 mL) were added sodium acetate (0.60 g, 7.34 mmol) and hydroxylamine hydrochloride (0.34 g, 4.90 mmol). The mixture was stirred at 25° C. for 1 h. The reaction solution was poured into ice water (20 mL) and extracted with ethyl acetate (20 mL×3). The combined organic layer was dried over anhydrous sodium sulfate, and filtered, and the filtrate was concentrated under reduced pressure to give the residue. The residue was purified by flash column chromatography (silica gel, 0-50% gradient of THF/petroleum ether) to give 2-(4,4-difluoropiperidin-1-yl)-6-methoxypyrimidin-4-amine (0.60 mg, 2.44 mmol, yield: 100%) as a yellow solid. LCMS (ESI): [M+H]⁺=245.1 Step 3: Potassium carbonate (3.14 g, 22.3 mmol) was added to a solution of 4-bromo-2-fluoronaphthalene-1-carboxylic acid (2.00 g, 7.43 mmol) and 6-azaspiro[2.5]octane hydrochloride (1.65 g, 11.1 mmol) in DMSO (40 mL). The mixture was heated to 120° C. and stirred for 72 h. The reaction mixture was diluted with water (50 mL), and the pH was adjusted to 4 with 3 M diluted hydrochloric acid, and extracted with ethyl acetate (50 mL×2). The combined organic layer was dried over anhydrous sodium sulfate, and filtered, and the filtrate was concentrated under reduced pressure to give the residue. The residue was purified by flash column chromatography (silica gel, 0-15% gradient of ethyl acetate/petroleum ether) to give 2-{6-azaspiro[2.5]octan-6-yl}-4-bromonaphthalene-1-carboxylic acid as a yellow oil (0.65 g, 1.80 mmol, yield: 24%). LCMS (ESI): [M+H]⁺=361.9.

Step 4: Oxalyl chloride (107 mg, 0.83 mmol) was added to the mixture of 2-{6-azaspiro[2.5]octan-6-yl}-4-bromonaphthalene-1-carboxylic acid (250 mg, 0.69 mmol) in dichloromethane (5 mL), N,N-dimethylformamide (5.07 mg, 0.07 mmol) was added dropwise to the reaction solution, and the mixture was stirred at 25° C. for 1 h. The reaction solution was concentrated and concentrated under reduced pressure to give 2-{6-azaspiro[2.5]octan-6-yl}-4-bromonaphthalene-1-carbonyl chloride (250 mg, crude) as a yellow solid. LCMS (ESI): 376.0. (sent methanol samples to get the mass of methyl ester) Step 5: N,N-diisopropylethylamine (313 mg, 2.37 mmol) was added to a solution of 2-(4,4-difluoropiperidin-1-yl)-6-methoxypyrimidin-4-amine (193 mg, 0.79 mmol) and 2-{6-azaspiro[2.5]octan-6-yl}-4-bromonaphthalene-1-carbonyl chloride (300 mg, 0.79 mmol) in THE (6 mL). The mixture was stirred at 50° C. for 16 h. The reaction solution was diluted with water (20 mL) and extracted with ethyl acetate (20 mL×3). The combined organic layer was dried over anhydrous sodium sulfate, and filtered, and the filtrate was concentrated under reduced pressure to give the residue. The residue was purified by flash column chromatography (silica gel, 0-10% gradient of THF/petroleum ether) to give 2-{6-azaspiro[2.5]octan-6-yl}-4-bromo-N-[2-(4,4-difluoropiperidin-1-yl)-6-methoxypyrimidin-4-yl]naphthalene-1-carboxamide (80 mg, 0.13 mmol, yield: 17.2%) as a yellow solid. LCMS (ESI): [M+H]⁺=588.0.

Step 6: 2-{6-azaspiro[2.5]octan-6-yl}-4-bromo-N-[2-(4,4-difluoropiperidin-1-yl)-6-methoxypyrimidin-4-yl]naphthalene-1-carboxamide (80 mg, 0.13 mmol) and 2-hydroxyethanesulfonamide (34.1 mg, 0.27 mmol) were dissolved in dioxane (4 mL), and potassium phosphate (86.8 mg, 0.41 mmol), 2-di-tert-butylphosphine-2′,4′,6′-triisopropylbiphenyl (5.79 mg, 0.01 mmol) and tris(dibenzylideneacetone)dipalladium (12.7 mg, 0.01 mmol) were added. The solution was stirred at 60° C. for 16 h under nitrogen atmosphere. The reaction solution was filtered, diluted, and purified by preparative HPLC (C18, 50-90% gradient of water (formic acid)/acetonitrile) to give 2-{6-azaspiro[2.5]octan-6-yl}-N-[2-(4,4-difluoropiperidin-1-yl)-6-methoxypyrimidin-4-yl]-4-(2-hydroxyethanesulfonamido)naphthalene-1-carboxamide (9.3 mg, 0.01 mmol, yield: 11%) as a white solid. LCMS (ESI): [M+H]⁺=631.5; ¹H NMR (400 MHz, DMSO-d₆) δ ppm 10.70 (br s, 1H), 10.02-9.64 (m, 1H), 8.25 (br d, J=8.4 Hz, 1H), 7.90 (br dd, J=2.0, 4.8 Hz, 1H), 7.63-7.39 (m, 3H), 7.00 (br s, 1H), 5.20-4.82 (m, 1H), 4.01-3.77 (m, 9H), 3.46-3.38 (m, 2H), 3.09 (br s, 4H), 1.98 (br s, 4H), 1.40 (br s, 4H), 0.30 (s, 4H)

### Example 8

### N-(2-(4,4-difluoropiperidin-1-yl)-6-methylpyrimidin-4-yl)-5-((2-hydroxyethyl)sulfonamido)-7-(6-azaspiro[2.5]octan-6-yl)quinoline-8-carboxamide

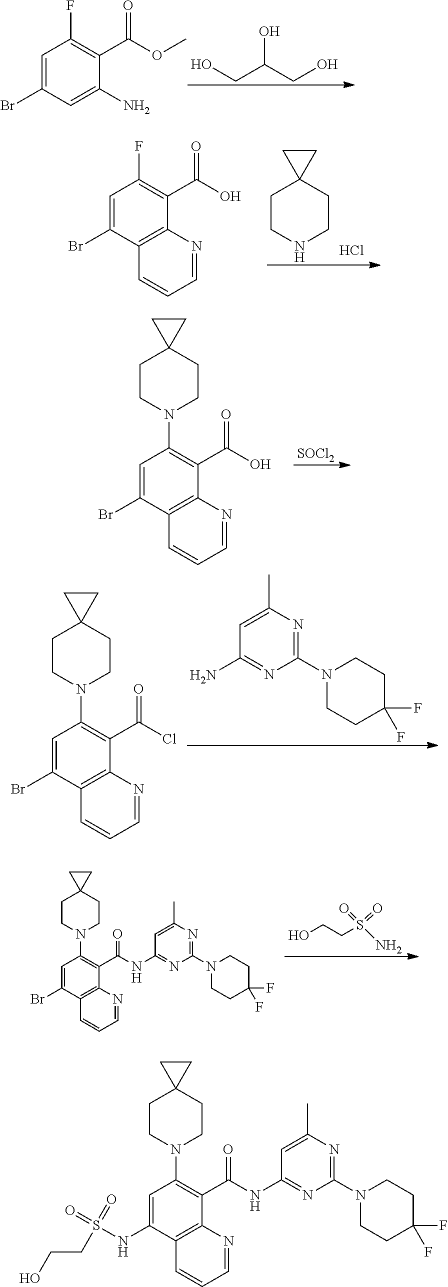

Step 1: The mixed solution of glycerol (1.13 g, 12.09 mmol) and sodium 3-nitrobenzenesulfonate (1.00 g, 4.43 mmol) in sulfuric acid (4.00 mL)/water (2.00 mL) was stirred at 120° C. for 30 min. The reaction system was cooled to 40° C. Methyl 2-amino-4-bromo-6-fluorobenzoate (1.00 g, 4.03 mmol) was added to the reaction mixture, which was stirred at 120° C. for 4 h. The reaction mixture was poured into ice water (50 mL) and filtered, and the cake was washed with ethyl acetate (30 mL). The organic phase was discarded, the pH of the aqueous phase was adjusted to 9 with potassium carbonate solution, and extraction was performed with ethyl acetate (15 mL×2). The organic phase was discarded, the pH of the aqueous phase was adjusted to 4 with diluted hydrochloric acid (1 M), and extraction was performed with ethyl acetate (15 mL×3). The combined organic layer was dried, filtered, and concentrated to give 5-bromo-7-fluoroquinoline-8-carboxylic acid (200 mg, 0.74 mmol, yield: 16.7%) as a gray solid. LCMS (ESI): [M+H]⁺=270.0; ¹H NMR (400 MHz, DMSO) δ 14.02 (s, 1H), 9.07 (dd, J=4.3, 1.6 Hz, 1H), 8.60 (dd, J=8.5, 1.6 Hz, 1H), 8.21 (d, J=9.0 Hz, 1H), 7.85-7.74 (m, 1H).

Step 2: Potassium carbonate (1.56 g, 11.10 mmol) was added to a solution of 5-bromo-7-fluoroquinoline-8-carboxylic acid (1.00 g, 3.70 mmol) and 6-azaspiro[2.5]octane hydrochloride (0.83 g, 5.55 mmol) in DMSO (13.00 mL) at 20° C. The reaction mixture was stirred at 120° C. for 16 h. The reaction mixture was filtered, and the cake was stirred in water (20 mL) for 5 min, filtered again, and dried to give 7-(6-azaspiro[2.5]octan-6-yl)-5-bromoquinoline-8-carboxylic acid (900 mg, 2.49 mmol, yield: 67.4%) as a yellow solid. LCMS (ESI): [M+H]⁺=361.1; ¹H NMR (400 MHz, CD₃OD) δ 8.78 (dd, J=4.3, 1.7 Hz, 1H), 8.42 (dd, J=8.4, 1.7 Hz, 1H), 7.71 (d, J=1.6 Hz, 1H), 7.40 (ddd, J=8.5, 4.3, 1.5 Hz, 1H), 3.35 (t, J=5.4 Hz, 4H), 1.57 (t, J=5.3 Hz, 4H), 0.36 (d, J=1.6 Hz, 4H).

Step 3: Thionyl chloride (100 μL) was added to a solution of 7-(6-azaspiro[2.5]octan-6-yl)-5-bromoquinoline-8-carboxylic acid (0.53 g, 1.47 mmol) in dichloromethane (3.00 mL) at 20° C. The reaction mixture was stirred at 50° C. for 1 h. The reaction mixture was concentrated to dryness without purification to give 7-(6-azaspiro[2.5]octan-6-yl)-5-bromoquinoline-8-carboxylic acid chloride (550 mg, 1.45 mmol, yield: 98.7%) as an orange solid. LCMS (ESI): [M+H]⁺=377.1

Step 4: Potassium tert-butoxide (328 mg, 2.90 mmol) was added to a solution of 2-(4,4-difluoropiperidyl)-6-methylpyrimidin-4-ylamine (330.63 mg, 1.45 mmol) in THE (10.00 mL) at 20° C. The reaction mixture was stirred for 5 min at 20° C. and 7-(6-azaspiro[2.5]octan-6-yl)-5-bromoquinoline-8-carboxylic acid chloride (550 mg, 1.45 mmol) was added. The reaction mixture was stirred at 20° C. for 16 h. The reaction mixture was diluted with water (20 mL) and extracted with ethyl acetate (10 mL×3). The organic phase was washed with saturated sodium chloride solution (20 mL×2), dried, filtered, concentrated to give the residue, which was purified by flash column chromatography (silica, 0-20% ethyl acetate/petroleum ether) to give [7-(6-azaspiro[2.5]octan-6-yl)-5-bromo(8-quinolinium)]-N-[2-(4,4-difluoropiperidyl)-6-methylpyrimidin-4-yl]carboxamide (250 mg, 0.44 mmol, yield: 30%), LCMS (ESI): [M+H]⁺=573.3

Step 5: To a mixture of [7-(6-azaspiro[2.5]octan-6-yl)-5-bromo(8-quinolinium)]-N-[2-(4,4-difluoropiperidyl)-6-methylpyrimidin-4-yl]carboxamide (150 mg, 0.26 mmol), 2-hydroxyethylsulfonamide (98 mg, 0.79 mmol), potassium phosphate (170 mg, 0.79 mmol), tris(dibenzylideneacetone) dipalladium (24 mg, 0.03 mmol), and 2-di-tert-butylphosphine-2′,4′,6′-triisopropylbiphenyl (22 mg, 0.052 mmol) was added 1,4-dioxane (7.00 mL) at 20° C., and the resulting reaction mixture was subjected to nitrogen bubbling for 2 min and stirred at 60° C. for 5 h. Ethyl acetate (20 mL) was added to the reaction mixture, the mixture was filtered, and the filtrate was washed with saturated sodium chloride solution (10 mL×3). The organic phase was dried, filtered, and concentrated to give the residue, which was purified by flash column chromatography (silica, 0-5% gradient of methanol/dichloromethane) to give 100 mg of the product. Then, the product was further purified by preparative HPLC (C18, 70-90% gradient of acetonitrile/water) to give N-(2-(4,4-difluoropiperidin-1-yl)-6-methylpyrimidin-4-yl)-5-((2-hydroxyethyl)sulfonamido)-7-(6-azaspiro[2.5]octan-6-yl)quinoline-8-carboxamide (61 mg, 0.10 mmol. yield: 38%) as an orange solid. LCMS (ESI): [M+H]⁺=616.4; ¹H NMR (400 MHz, DMSO) δ 10.60 (s, 1H), 9.95 (s, 1H), 8.81 (s, 1H), 8.58 (d, J=8.6 Hz, 1H), 7.53-7.43 (m, 3H), 5.03 (s, 1H), 3.83 (d, J=14.7 Hz, 6H), 3.31 (s, 2H), 3.21 (s, 4H), 2.34 (s, 3H), 1.95 (s, 4H), 1.40 (s, 4H), 0.31 (s, 4H).

### Example 9

### N-(2-(4,4-difluoropiperidin-1-yl)-6-methylpyrimidin-4-yl)-7-((2-hydroxyethyl)sulfonamido)-5-(6-azaspiro[2.5]oct-6-yl)-2,3-dihydro-1H-indene-4-carboxamide

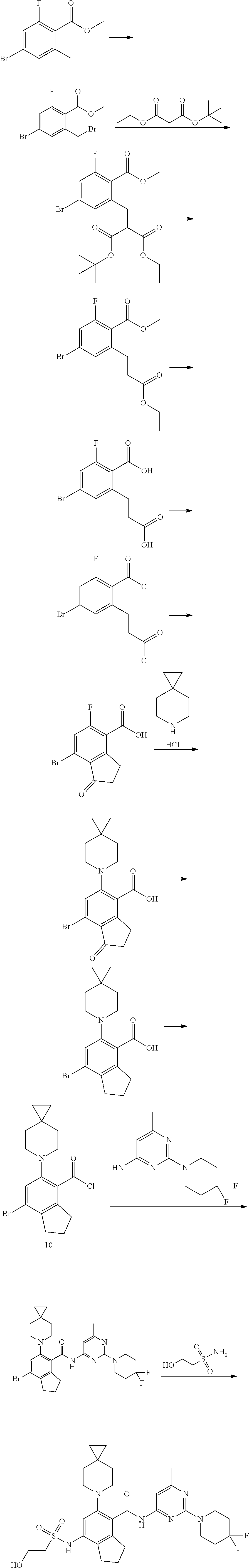

Step 1: The mixed solution containing methyl 4-bromo-2-fluoro-6-methylbenzoate (25.0 g, 101.19 mmol), N-bromosuccinimide (21.6 g, 121.43 mmol), azobisisobutyronitrile (8.5 g, 50.60 mmol) and acetonitrile (250 mL) was stirred at 70° C. for 3 h. The reaction solution was poured into water (50 mL) and extracted with ethyl acetate (50 mL×3), and the organic phase was dried over anhydrous sodium sulfate, filtered, concentrated, and purified by column chromatography (silica gel, 0-10% gradient of ethyl acetate/petroleum ether) to give methyl 4-bromo-2-(bromomethyl)-6-fluorobenzoate (18.1 g, 55.53 mmol, yield: 54.9%) as a colorless oil compound. LCMS (ESI): [M+H]⁺=326.9; ¹H NMR (400 MHz, DMSO) δ 7.73 (m, 2H), 4.77 (s, 2H), 3.91 (s, 3H).

Step 2: The mixed solution containing methyl 4-bromo-2-(bromomethyl)-6-fluorobenzoate (18 g, 55.22 mmol), tert-butyl ethyl malonate (11.4 g, 60.74 mmol), cesium carbonate (36.7 g, 110.44 mmol) and N,N-dimethylformamide (200 mL) was stirred at 20° C. for 2 h. The reaction solution was poured into water (300 mL) and extracted with ethyl acetate (100 mL×3), and the organic phase was dried over anhydrous sodium sulfate, filtered, concentrated, and purified by column chromatography (silica gel, 0-10% gradient of ethyl acetate/petroleum ether) to give 1-(tert-butyl)3-ethyl-2-(5-bromo-3-fluoro-2-(methoxycarbonyl)benzyl)malonic acid (15.4 g, crude) as a colorless oil compound. LCMS (ESI): [M+Na]+=455.3; ¹H NMR (400 MHz, DMSO) δ 7.64 (dd, J=9.6, 2.0 Hz, 1H), 7.47-7.44 (m, 1H), 4.17-4.08 (m, 2H), 3.88 (s, 3H), 3.70 (t, J=8.0 Hz, 1H), 3.18-3.04 (m, 2H), 1.35 (s, 9H), 1.15 (t, J=7.2 Hz, 3H).

Step 3: The solution containing 1-(tert-butyl)3-ethyl-2-(5-bromo-3-fluoro-2-(methoxycarbonyl)benzyl)malonic acid (15 g, 34.62 mmol) and trifluoroacetic acid (60 mL) was stirred at 20° C. for 1 h, the reaction solution was concentrated to remove trifluoroacetic acid, then toluene (60 mL) and triethylamine (35.75 g, 346.21 mmol) were added, and the mixture was continued to be stirred at 120° C. for 3 h. The reaction solution was concentrated, saturated aqueous sodium bicarbonate solution was added to adjust the pH to be close to 8, and then the mixture was extracted with ethyl acetate (50 mL×3). The organic phase was dried over anhydrous sodium sulfate, filtered, concentrated, and purified by column chromatography (silica gel, 0-10% gradient of ethyl acetate/petroleum ether) to give the compound methyl 4-bromo-2-(3-ethoxy-3-oxopropyl)-6-fluorobenzoate (6.8 g, 20.41 mmol, yield: 59.0%). LCMS (ESI): [M+H]⁺=333.0.

Step 4: The mixed solution containing methyl 4-bromo-2-(3-ethoxy-3-oxopropyl)-6-fluorobenzoate (6.8 g, 20.41 mmol), lithium hydroxide monohydrate (4.9 g, 204.11 mmol), THE (40 mL), methanol (40 mL) and water (40 mL) was stirred at 20° C. for 4 h. The reaction solution was concentrated to remove THE and methanol, 4 M hydrochloric acid ethyl acetate solution (100 mL) was added to adjust the pH to be close to 5, and then the mixture was extracted with ethyl acetate (20 mL×3). The organic phase was dried over anhydrous sodium sulfate, filtered, and concentrated to give 4-bromo-2-(2-carboxyethyl)-6-fluorobenzoic acid (5.1 g, 17.52 mmol, yield: 85.8%) as a white solid compound. LCMS (ESI): [M+Na]+=313.0; ¹H NMR (400 MHz, DMSO) δ 12.95 (s, 2H), 7.58 (dd, J=9.2, 2.0 Hz, 1H), 7.49 (d, J=2.0 Hz, 1H), 2.94 (dd, J=8.4, 7.2 Hz, 2H), 2.60 (dd, J=8.4, 7.2 Hz, 2H).

Step 5: Oxalyl chloride (15.3 g, 120.25 mmol) was added to a mixed solution containing 4-bromo-2-(2-carboxyethyl)-6-fluorobenzoic acid (5.0 g, 17.18 mmol) and dichloromethane (50 mL), and the resulting mixture was stirred at 20° C. for 1 h. The reaction solution was concentrated to give 4-bromo-2-(3-chloro-3-oxopropyl)-6-fluorobenzoyl chloride (5.7 g, crude) as a yellow oil compound. LCMS (ESI): [M+H]⁺=319.0 (corresponding methyl ester).

Step 6: In dichloromethane (50 mL) was dissolved 4-bromo-2-(3-chloro-3-oxopropyl)-6-fluorobenzoyl chloride (5.0 g, 15.25 mmol), the solution was added dropwise at 0° C. to a solution containing aluminum trichloride (8.1 g, 60.98 mmol) and dichloromethane (50 mL), and the reaction solution was stirred at 40° C. for 16 h. The reaction mixture was poured into water (30 mL) and extracted with dichloromethane (30 mL×3), and the organic phase was dried over anhydrous sodium sulfate, filtered, concentrated, and purified by column chromatography (silica gel, 0-100% gradient of ethyl acetate/petroleum ether) to give 7-bromo-5-fluoro-1-oxo-2,3-dihydro-1H-indene-4-carboxylic acid (3.4 g, 12.45 mmol, yield: 81.7%) as a brown solid compound, LCMS (ESI): [M+H]⁺=273.0.

Step 7: The mixed solution containing 7-bromo-5-fluoro-1-oxo-2,3-dihydro-1H-indene-4-carboxylic acid (3.3 g, 12.09 mmol), 6-azaspiro[2.5]octane hydrochloride (2.7 g, 18.13 mmol), potassium carbonate (6.0 g, 42.30 mmol) and DMSO (30 mL) was stirred at 100° C. for 12 h. The reaction solution was poured into water (30 mL) and extracted with ethyl acetate (30 mL×3), and the organic phase was dried over anhydrous sodium sulfate, filtered, concentrated, and purified by column chromatography (silica gel, 0-100% gradient of ethyl acetate/petroleum ether) to give 7-bromo-1-oxo-5-(6-azaspiro[2.5]oct-6-yl)-2,3-dihydro-1H-indene-4-carboxylic acid (2.5 g, 6.86 mmol, yield: 56.8%) as a gray solid compound, LCMS (ESI): [M+H]⁺=364.4.

Step 8: The mixed solution containing 7-bromo-1-oxo-5-(6-azaspiro[2.5]oct-6-yl)-2,3-dihydro-1H-indene-4-carboxylic acid (1 g, 2.75 mmol), triethylsilane (3.2 g, 27.45 mmol) and trifluoroacetic acid (10 mL) was stirred at 80° C. for 12 h. The reaction solution was concentrated, aqueous sodium bicarbonate solution (50 mL) was added, then 4 M hydrochloric acid ethyl acetate solution (40 mL) was added to adjust the pH to be close to 4, and the mixture was extracted with ethyl acetate (30 mL×3). The organic layer was dried over anhydrous sodium sulfate, filtrated, concentrated, and purified by column chromatography (silica gel, 0-100% gradient of ethyl acetate/petroleum ether) to give 7-bromo-5-(6-azaspiro[2.5]oct-6-yl)-2,3-dihydro-1H-indene-4-carboxylic acid (700 mg, 2.00 mmol, yield: 72.8%) as a white solid compound. LCMS (ESI): [M+H]⁺=350.2.

Step 9: Oxalyl chloride (518 mg, 4.00 mmol) was added to a mixed solution containing 7-bromo-5-(6-azaspiro[2.5]oct-6-yl)-2,3-dihydro-1H-indene-4-chlorinated phthalic acid derivative of carboxylic acid (700 mg, 2.00 mmol) and dichloromethane (10 mL), and the resulting mixture was stirred at 20° C. for 30 min and concentrated to give 7-bromo-5-(6-azaspiro[2.5]oct-6-yl)-2,3-dihydro-1H-indene-4-carbonyl chloride (760 mg, crude) as a yellow oil compound. LCMS (ESI): [M+H]⁺=364.2. (corresponding methyl ester)

Step 10: The mixed solution containing 2-(4,4-difluoropropiperidinyl)-6-methylpyrimidine-4-amine (650 mg, 2.85 mmol), N,N-diisopropylethylamine (2.5 g, 18.99 mmol), potassium tert-butoxide (323 mg, 2.85 mmol) and THE (10 mL) was stirred at 20° C. for 30 min. Then 7-bromo-5-(6-azaspiro[2.5]oct-6-yl)-2,3-dihydro-1H-indene-4-carbonyl chloride (700 mg, 1.90 mmol) was added, the reaction solution was poured into water (30 mL) and extracted with ethyl acetate (30 mL×3), and the organic phase was dried over anhydrous sodium sulfate, filtered, concentrated, and purified by column chromatography (silica gel, 0-50% gradient of ethyl acetate/petroleum ether) to give 7-bromo-N-(2-(4,4-difluoropiperidin-1-yl)-6-methylpyrimidin-4-yl)-5-(6-azaspiro[2.5]oct-6-yl)-2,3-dihydro-1H-indene-4-carboxamide (630 mg, 1.12 mmol, yield: 59.2%) as a gray solid compound. LCMS (ESI): [M+H]⁺=560.3.

Step 11: The reaction solution containing 7-bromo-N-(2-(4,4-difluoropiperidin-1-yl)-6-methylpyrimidin-4-yl)-5-(6-azaspiro[2.5]oct-6-yl)-2,3-dihydro-1H-indene-4-carboxamide (300 mg, 0.54 mmol), 2-hydroxyethanesulfonamide (137 mg, 1.07 mmol), potassium phosphate (348 mg, 1.61 mmol), 2-di-tert-butylphosphino-2′,4′,6′-triisopropylbiphenyl (23 mg, 0.054 mmol), tris(dibenzylideneacetone) dipalladium (50 mg, 0.054 mmol) and dioxane (5 mL) was stirred at 100° C. for 8 h under nitrogen atmosphere. The reaction solution was poured into water (30 mL) and extracted with ethyl acetate (30 mL×3), and the organic phase was dried over anhydrous sodium sulfate, filtered, concentrated, and purified by column chromatography (silica gel, 0-100% gradient of ethyl acetate/petroleum ether) to give N-(2-(4,4-difluoropiperidin-1-yl)-6-methylpyrimidin-4-yl)-7-((2-hydroxyethyl)sulfonamido)-5-(6-azaspiro[2.5]oct-6-yl)-2,3-dihydro-1H-indene-4-carboxamide (149.0 mg, 0.25 mmol, yield: 46.0%) as a white solid compound. LCMS (ESI): [M+H]⁺=605.40; 1H NMR (400 MHz, CDCl3) δ 12.79 (s, 1H), 7.46 (s, 1H), 7.39 (s, 1H), 6.61 (s, 1H), 4.12 (q, J=5.2 Hz, 2H), 3.98 (t, J=5.6 Hz, 4H), 3.45-3.37 (m, 4H), 3.04 (t, J=5.2 Hz, 4H), 2.79 (t, J=7.6 Hz, 2H), 2.48 (t, J=5.6 Hz, 1H), 2.37 (s, 3H), 2.13 (q, J=7.6 Hz, 2H), 2.03-1.92 (m, 4H), 1.79-1.56 (m, 4H), 0.38 (s, 4H).

### Example 10

### N-[2-amino-4-(4,4-difluoropiperidin-1-yl)-5-fluoro-1,3-benzothiazol-6-yl]-2-{6-azaspiro[2.5]octan-6-yl}-4-(1-hydroxypropane-2-sulfonamido)naphthalene-1-carboxamide

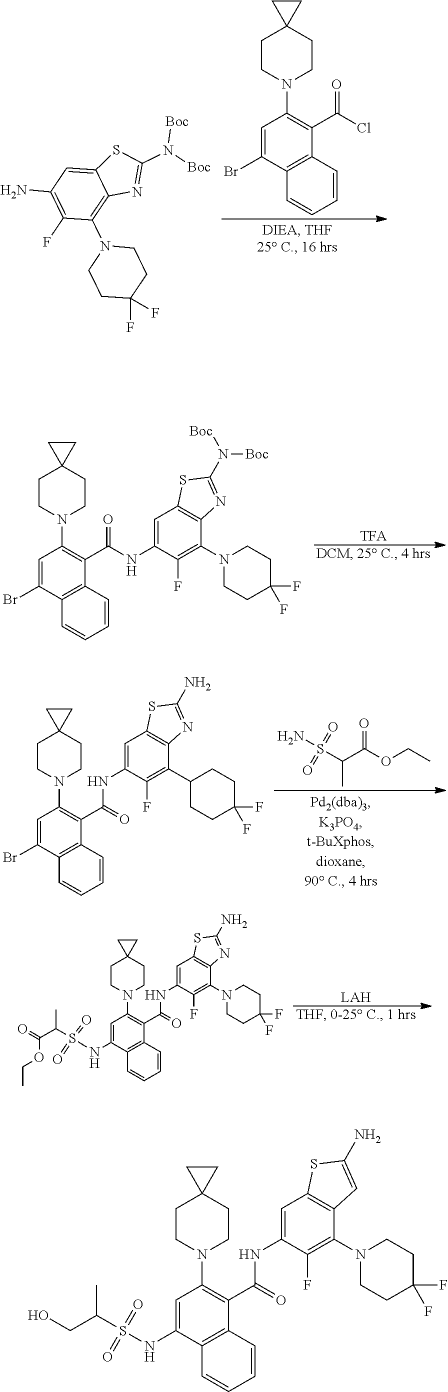

Step 1: N,N-diisopropylethylamine (418 mg, 3.17 mmol) was added to a solution of tert-butyl N-[6-amino-4-(4,4-difluoropiperidin-1-yl)-5-fluoro-1,3-benzothiazol-2-yl]-N-[(tert-butoxy)carbonyl]carbamate (120 mg, 0.30 mmol) and 2-{6-azaspiro[2.5]octan-6-yl}-4-bromonaphthalene-1-carbonyl chloride (716 mg, 1.43 mmol) in THF (8.00 mL), and the mixture was stirred at 25° C. for 16 h. The reaction solution was diluted with water (5 mL) and extracted with dichloromethane (15 mL×2). The organic layer was washed with saturated saline (20 mL), dried over magnesium sulfate, filtered, and concentrated under reduced pressure. The residue was purified by flash column chromatography (silica gel, 0-20% gradient of ethyl acetate/petroleum ether) to give tert-butyl N-[6-(2-{6-azaspiro[2.5]octan-6-yl}-4-bromonaphthalene-1-ylamino)-4-(4,4-difluoropiperidin-1-yl)-5-fluoro-1,3-benzothiazol-2-yl]-N-[(tert-butoxy)carbonyl]carbamate (650 mg, 0.77 mmol, yield: 72.8%) as a yellow solid. LCMS (ESI): [M+H]⁺=846.1

Step 2: Tert-butyl N-[6-(2-{6-azaspiro[2.5]octan-6-yl}-4-bromonaphthalene-1-ylamino)-4-(4,4-difluoropiperidin-1-yl)-5-fluoro-1,3-benzothiazol-2-yl]-N-[(tert-butoxy)carbonyl]carbamate (400 mg, 0.47 mmol) was added to a mixed solution of trifluoroacetic acid (0.50 mL) and dichloromethane (1.50 mL). The mixture was stirred at 25° C. for 4 h. The reaction solution was washed with saturated aqueous sodium bicarbonate solution (20 mL) and extracted with dichloromethane (20 mL×2). The combined organic layer was dried over anhydrous magnesium sulfate, and filtered, and the filtrate was concentrated under reduced pressure to give N-[2-amino-4-(4,4-difluoropiperidin-1-yl)-5-fluoro-1,3-benzothiazol-6-yl]-2-{6-azaspiro[2.5]octan-6-yl}-4-bromonaphthalene-1-carboxamide (300 mg, 0.464 mmol, yield: 98.3%) as a white solid. LCMS (ESI): [M+H]⁺=645.8

Step 3: Ethyl 2-sulfamoylpropyl ester (101 mg, 0.56 mmol), potassium phosphate (363 mg, 1.68 mmol), di-tert-butyl-[2-(2,4,6-triisopropylphenyl) phenyl]phosphine (23.7 mg, 0.06 mmol) and tris(dibenzylideneacetone) dipalladium (52.2 mg, 0.06 mmol) were added to a solution of N-[2-amino-4-(4,4-difluoropiperidin-1-yl)-5-fluoro-1,3-benzothiazol-6-yl]-2-{6-azaspiro[2.5]octan-6-yl}-4-bromonaphthalene-1-carboxamide (360 mg, 0.56 mmol) in dioxane (2 mL) at 25° C. The reaction mixture was heated to 90° C. under nitrogen atmosphere and stirred for 4 h. The reaction mixture was concentrated under reduced pressure to give a residue. The residue was purified by flash column chromatography (silica gel, 0-30% gradient of THF/petroleum ether) to give ethyl 2-[(4-{[2-amino-4-(4,4-difluoropiperidin-1-yl)-5-fluoro-1,3-benzothiazol-6-yl]aminocarbonyl}-3-{6-azaspiro[2.5]octan-6-yl}naphthalen-1-yl)sulfamoyl]propyl ester (300 mg, 0.463 mmol, yield: 83%) as a white solid compound. LCMS (ESI): [M+H]⁺=745.2

Step 4: Ethyl 2-[(4-{[2-amino-4-(4,4-difluoropiperidin-1-yl)-5-fluoro-1,3-benzothiazol-6-yl]aminocarbonyl}-3-{6-azaspiro[2.5]octan-6-yl}naphthalen-1-yl)sulfamoyl]propyl ester (300 mg, 0.40 mmol) was added to a solution of THE (14 mL), lithium tetrahydridoaluminate (21.6 mg, 0.60 mmol) was added portionwise at 25° C., and the mixture was stirred at 25° C. for 1 h. The reaction solution was quenched water (0.05 mL) and 15% aqueous sodium hydroxide solution (0.05 mL) and filtered with water (0.1 mL), filtered, and the filtrate was concentrated under reduced pressure to give the residue. The residue was diluted with N,N-dimethylformamide (5 mL). The diluent was purified by preparative HPLC (C18, 28-68% gradient of water (formic acid)/acetonitrile) to give N-[2-amino-4-(4,4-difluoropiperidin-1-yl)-5-fluoro-1,3-benzothiazol-6-yl]-2-{6-azaspiro[2.5]octan-6-yl}-4-(1-hydroxypropane-2-sulfonamido)naphthalene-1-carboxamide (37.9 mg, 0.052 mmol, yield: 13%) as a white solid compound. LCMS (ESI): [M+H]⁺=703.1; ¹H NMR (400 MHz, DMSO-d₆) δ ppm 10.01 (s, 1H), 9.95-9.61 (m, 1H), 8.27 (d, J=8.4 Hz, 1H), 7.94 (d, J=8.4 Hz, 1H), 7.84 (d, J=6.4 Hz, 1H), 7.62-7.44 (m, 5H), 5.32-4.83 (m, 1H), 3.92 (dd, J=4.4, 11.2 Hz, 1H), 3.50 (dd, J=8.0, 11.2 Hz, 1H), 3.40 (br t, J=5.2 Hz, 4H), 3.27-3.20 (m, 1H), 3.13 (br t, J=5.2 Hz, 4H), 2.21-2.03 (m, 4H), 1.46 (br s, 4H), 1.34 (d, J=7.2 Hz, 3H), 0.33 (s, 4H)

### Example 11

### N-(2-(4,4-difluoropiperidin-1-yl)-6-methylpyrimidin-4-yl)-7-((2-hydroxy-1,1-dimethylethyl)sulfonamide)-5-(6-azaspiro[2.5]oct-6-yl)-2,3-dihydro-1H-indene-4-carboxamide

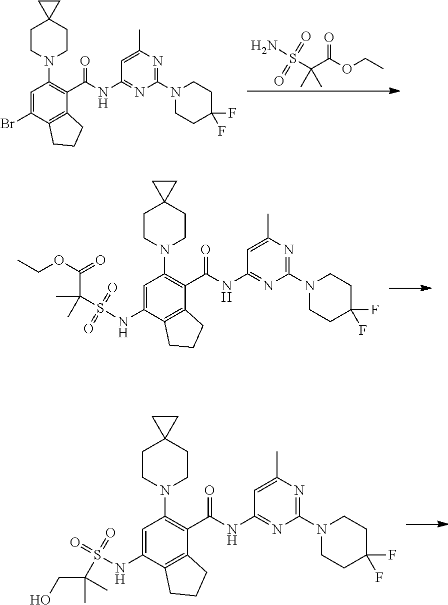 [SMILES: Cc1cc(NC(=O)c2c(N3CCC4(CC3)CC4)cc(Br)c3c2CCC3)nc(N2CCC(F)(F)CC2)n1 ; CCOC(=O)C(C)(C)S(N)(=O)=O ; CCOC(=O)C(C)(C)S(=O)(=O)Nc1cc(N2CCC3(CC2)CC3)c(C(=O)Nc2cc(C)nc(N3CCC(F)(F)CC3)n2)c2c1CCC2 ; Cc1cc(NC(=O)c2c(N3CCC4(CC3)CC4)cc(NS(=O)(=O)C(C)(C)CO)c3c2CCC3)nc(N2CCC(F)(F)CC2)n1]

Step 1: The solution containing 7-bromo-N-(2-(4,4-difluoropiperidin-1-yl)-6-methylpyrimidin-4-yl)-5-(6-azaspiro[2.5]oct-6-yl)-2,3-dihydro-1H-indene-4-carboxamide (300 mg, 0.54 mmol), ethyl 2-methyl-2-sulfopropanoate (209 mg, 1.07 mmol), potassium phosphate (348 mg, 1.61 mmol), 2-di-tert-butylphosphino-2′,4′,6′-triisopropylbiphenyl (23 mg, 0.054 mmol), tris(dibenzylideneacetone) dipalladium (50 mg, 0.054 mmol) and dioxane (5 mL) was stirred at 100° C. for 8 h under nitrogen atmosphere. The reaction solution was poured into water (30 mL) and extracted with ethyl acetate (30 mL×3), and the organic phase was dried over anhydrous sodium sulfate, filtered, concentrated, and purified by column chromatography (silica gel, 0-30% gradient of ethyl acetate/petroleum ether) to give ethyl 2-(N-(7-((2-(4,4-difluoropiperidin-1-yl)-6-methylpyrimidin-4-yl)carbamoyl)-6-(6-azaspiro[2.5]oct-6-yl)-2,3-dihydro-1H-inden-4-yl)sulfamoyl)-2-methylpropionate (270 mg, 0.40 mmol, yield: 76.2%) as a pale yellow solid compound. LCMS (ESI): [M+H]⁺=675.5.

Step 2: Lithium tetrahydridoaluminate (40 mg, 1.11 mmol) was added in portions to a mixed solution containing ethyl 2-(N-(7-((2-(4,4-difluoropiperidin-1-yl)-6-methylpyrimidin-4-yl)carbamoyl)-6-(6-azaspiro[2.5]oct-6-yl)-2,3-dihydro-1H-inden-4-yl)sulfamoyl)-2-methylpropionate (250 mg, 0.37 mmol) and THF (5 mL). The reaction solution was stirred at 20° C. for 1 h, quenched with saturated aqueous ammonium chloride solution (20 mL), and extracted with ethyl acetate (30 mL×3), and the organic phase was dried over anhydrous sodium sulfate, filtered, concentrated, and purified by column chromatography (silica gel, 0-100% gradient of ethyl acetate/petroleum ether and C18, 0-100% gradient of acetonitrile/water) to give N-(2-(4,4-difluoropiperidin-1-yl)-6-methylpyrimidin-4-yl)-7-((2-hydroxy-1,1-dimethylethyl)sulfonamide)-5-(6-azaspiro[2.5]oct-6-yl)-2,3-dihydro-1H-indene-4-carboxamide (40.25 mg, 0.064 mmol, yield: 17.2%) as a white solid compound. LCMS (ESI): [M+H]⁺=633.40; 1H NMR (400 MHz, DMSO) δ 13.05 (s, 1H), 9.17 (s, 1H), 7.46 (s, 1H), 7.38 (s, 1H), 5.22 (s, 1H), 3.90 (t, J=5.6 Hz, 4H), 3.55 (s, 2H), 3.23 (t, J=7.6 Hz, 2H), 2.94-2.85 (m, 6H), 2.30 (s, 3H), 2.02-1.91 (m, 6H), 1.66 (s, 4H), 1.23 (s, 6H), 0.36 (s, 4H).

### Effect Example 1: Enzymatic Activity Test

Materials: Human KIF18A (amino acid sequence 1-417) was purchased from Viva Biotech (Shanghai) Ltd.; the ADP-Glo™ protein kinase kit was acquired from Promega, USA; tubulin was sourced from Cytoskeleton, USA; and the 384-well assay plate and Envision multifunctional microplate reader were obtained from PerkinElmer, USA.

Enzymatic Activity Test: Dissolve the compound powder in DMSO to prepare a stock solution of 10 mM. Then perform gradient dilution for the compounds in the microplate to achieve a final concentration of 0-10 M. Then, add 2.5 μL each of tubulin, compound, ATP, and KIF18A protein to the microplate in sequence for reaction at ambient temperature for 120 min. The final concentrations in the enzyme reaction are 60 μg/mL for microtubules, 25 μM for ATP, and 2.5 nM for KIF18A protein. After the enzyme reaction, add 10 μL of ADP-GLO reaction reagent to each well and incubate at ambient temperature for 30 min. After that, add L of detection reagent to each well and incubate at ambient temperature for 30 min in the dark. Finally, perform chemiluminescence detection using the PerkinElmer Envision.

TABLE 1
Enzymatic Activity Biological Data for
Compounds of the Present Invention

| | Enzyme |
|---|---|
| Example | Activity (nM) |
| 1 | 112 |
| 2 | 29 |
| 3 | 27 |
| 4 | 173 |
| 5 | 134 |
| 6 | 237 |
| 7 | 635 |
| 8 | 280 |
| 9 | 176 |
| 10 | 180 |
| 11 | 170 |

NT means no test

### Example 2: Cell Proliferation Activity Test

Materials and Cells: OVCAR3 cells were purchased from Nanjing Cobioer Biosciences Co., Ltd. RPMI-1640 medium, fetal bovine serum, and the CyQUANT Direct Cell Proliferation Assay kit were purchased from Thermo Fisher Scientific, USA; 96-well cell culture plates were sourced from Corning, USA.

Cell Culture: Culture OVCAR3 cells with RPMI-1640 medium containing 10% fetal bovine serum and incubate them in a 5% CO₂ incubator at 37° C. Only cells in the logarithmic growth phase can be used for the test.

### Cell Proliferation Activity Test:

Inoculate OVCAR3 cells into a 96-well cell culture plate at 90 μL per well and incubate overnight in a 5% CO₂ incubator at 37° C. Dissolve the compound powder in DMSO to prepare a stock solution of 10 mM. Then perform gradient dilution for the compounds in the microplate to achieve a final concentration of 0-10 μM. Add 10 μL of cell culture medium containing compounds to each well to make the final DMSO content 0.2%. Incubate the cell plates in a 5% CO₂ incubator at 37° C. for 3 days. Add 100 μL of CyQUANT detection reagent to each well, react at 37° C. for 60 min, and perform fluorescence detection using the PerkinElmer Envision.

TABLE 2
OVCAR-3 Cell Viability Biological Data
for Compounds of the Present Invention

| | OVCAR-3 Cell |
|---|---|
| Example | Viability_IC50_nM |
| 1 | 442 |
| 2 | 25 |
| 3 | 28 |
| 4 | 37 |
| 5 | 36 |
| 6 | 67 |
| 7 | 98 |
| 8 | 488 |
| 9 | 26 |
| 10 | 35 |
| 11 | 20 |

NT means no test

### Example 3: CyQuant Cell Proliferation Activity Test

Materials and Cells: HT29 cells were purchased from Nanjing Cobioer Biosciences Co., Ltd.; RPMI-1640 medium, fetal bovine serum, Trypsin-EDTA (0.25%), 96-well plates, and CyQuant reagents were purchased from ThermoFisher (USA); DMSO was purchased from SIGMA (USA).

Cell Culture: Culture HT29 cells with RPMI-1640 containing 10% fetal bovine serum under the condition of 37° C. and 5% CO₂. Only cells in the logarithmic growth phase can be used for the test.

Cell Proliferation Activity Test: Use the CyQuant reagent to detect the proliferation inhibitory activity of compounds on HT29 cells. The cell density was adjusted, and 100 μL per well was inoculated into a 96-well plate (HT29 at 2000 cells per well), then incubated overnight at 37° C. and 5% CO₂. Add compounds of various target concentrations (initial concentration of 3000 nM, 3-fold dilution, 9 concentration gradients), and make the DMSO content 0.2%. The cell plate was incubated at 37° C. in 5% CO₂ for 3 days. Incubate with CyQuant reagent for 1 h, read the plate by Envision, and calculate IC₅₀ using XLFIT.

TABLE 3
HT-29 Cell Viability Biological Data for
Compounds of the Present Invention

| | HT-29 Cell |
|---|---|
| Example | Viability_IC50_nM |
| 1 | 877 |
| 2 | 50 |
| 3 | 45 |
| 4 | 52 |
| 5 | 65 |
| 6 | 74 |
| 7 | 123 |
| 8 | >1000 |
| 9 | 37 |
| 10 | 44 |
| 11 | 29 |

NT means no test

### Example 4: Experimental Testing of Cytotoxicity in Human Primary Hepatocytes

Materials: One donor for cytotoxicity testing. Information on human primary hepatocytes is provided in the table below.

| Donor | Race | Source |
|---|---|---|
| 1 | Caucasian | BioIVT |

Note:
Hepatocytes of other races may be used, and specific information will be reflected in the test report.

1) CellTiter-Glo Luminescent Cell Viability Assay System was Purchased from Promega (Madison, WI).

### Instruments:

| Instruments | Supplier | Cat# or Model |
|---|---|---|
| 96-well plate | Corning | 354407 |
| Centrifuge | Eppendorf | 5804R&5810R |
| CO₂ incubator | Thermo Scientific | 371 |
| Cell counter | Nexcelom Bioscience | Cellmeter K2 |
| | LLC | |
| Oscillator | IKA | MS3 BASIC |
| Microplate reader | TECAN | INFINITE 200 PRO |

### Experimental Design

Warm the following media to 37° C. in a water bath: Prepare hepatocyte recovery medium, inoculation medium, and incubation medium according to the table below.

| | Initial | Final | |
|---|---|---|---|
| Reagent | Concentration | Concentration | Volume |

Recovery Medium

| Williams' Medium E | — | | — | | 31.2 | mL |
|---|---|---|---|---|---|---|
| Isotonic Percoll | — | | 30% | | 13.5 | mL |
| DPBS (10×) | — | | — | | 1.5 | mL |
| GlutaMAX ™-1 (100×) | 200 mM/100× | | 2 | mM | 500 | μL |
| HEPES | 1M | | 15 | mM | 750 | μL |
| FBS | | | 5% | | 2.5 | mL |
| Human recombination | 4 | mg/mL | 4 | μg/mL | 50 | μL |
| insulin | | | | | | |
| Dexamethasone | 10 | mM | 1 | μM | 5 | μL |

Inoculation Medium

| Williams' Medium E | — | | — | | 45.7 | mL |
|---|---|---|---|---|---|---|
| FBS | — | | 5% | | 2.5 | mL |
| Dexamethasone | 10 | mM | 1 | μM | 5 | μL |
| Penicillin-streptomycin | Penicillin: 10,000 u/mL | | Penicillin: 100 u/mL | | 500 | μL |
| mixed solution | Streptomycin: 10,000 | | Streptomycin: 100 | | | |
| | μg/mL | | μg/mL | | | |
| Human recombination | 4 | mg/mL | 4 | μg/mL | 50 | μL |
| insulin | | | | | | |

| | | | | | |
|---|---|---|---|---|---|
| GlutaMAX ™-1 (100×) | 200 mM/100× | 2 | mM | 500 | μL |
| HEPES | 1M | 15 | mM | 750 | μL |

Incubation Medium

| Williams' Medium E | — | | — | | 48 | mL |
|---|---|---|---|---|---|---|
| Dexamethasone | 10 | mM | 0.1 | μM | 0.5 | μL |

| ITS (100×) | — | — | | 500 | μL |
|---|---|---|---|---|---|
| Penicillin-streptomycin | Penicillin: 10,000 u/mL | Penicillin: 50 u/mL | | 250 | μL |
| mixed solution | Streptomycin: 10,000 | Streptomycin: 50 | | | |
| | μg/mL | μg/mL | | | |
| GlutaMAX ™-1 (100×) | 200 mM/100× | 2 | mM | 500 | μL |
| HEPES | 1M | 15 | mM | 750 | μL |

1) Take one tube of cryopreserved hepatocytes and ensure that the hepatocytes are cryogenically frozen until recovery. The hepatocytes were quickly placed in a 37° C. water bath and gently shaken until all ice crystals were completely dissolved. After spraying with 75% ethanol, the tube was transferred to a biosafety cabinet.
2) Transfer the contents of the hepatocyte tube (1 mL, approximately 5×10⁶ cells) into a 50 mL centrifuge tube containing 50 mL of recovery medium and centrifuge at 100 g for 10 min. After centrifugation, aspirate the recovery medium and add sufficient inoculation medium to obtain a cell suspension with a cell density of approximately 1.0×10⁶ cells/mL. Count hepatocytes and determine viable cell density with the cell counter Cellometer©. Make sure that the viability of hepatocytes is greater than 80%. Adjust the cell density to 0.2×10⁶ cells/mL with inoculation medium, and inoculate into 96-well plates coated with collagen I at 100 μL per well. Incubate the plates at 37° C. for 4-6 h in a 5% CO₂ incubator with 95% relative humidity.
3) Prepare a 200× stock solution of the test compound in DMSO (the stock concentration of the test compound can be reduced depending on solubility). Make the final concentration of DMSO 0.5%
4) Prepare the working solutions on sterile 96-well plates by adding 2.5 μL of the test compound stock solution to 497.5 μL of hepatocyte culture medium.
5) Remove the hepatocyte culture medium from the cell plate, and add 125 μL of working solution to the corresponding wells in triplicate. Incubate the plate at 37° C. and 5% CO₂ for 72 h. After every 24 h of treatment, replace the medium in cell plates with freshly diluted test and positive control compounds from hepatocyte culture media. Return the plate to the incubator, and add 50 μL of CellTiter-Glo reagent directly into each well of the 96-well plate. Shake on a shaker for 10 min at ambient temperature. After 10 min, transfer 100 μL of the above incubation to a new white and opaque flat-bottom 96-well plate and record the fluorescence.

### Data Analysis:

All calculations were performed using Microsoft Excel.

The viability of the test compound can be calculated using the following formula:

$$\% Vehicle = \frac{{Read}_{Compound} - {Read}_{Blank}}{{Read}_{vehicle} - {Read}_{Blank}} \times 100 \%$$

The % Vehicle is fitted to the concentration of the test compound, and then GraphPad Prism 5.0 is used to model the data as a sigmoidal dose-response curve with a variable slope. The IC₅₀ of this compound is calculated from the curve using the following formula

$$Y = Bottom + ( Top - Bottom ) / ( 1 + 10 \hat{} ( ( Log IC 50 - X ) * HillSlope ) )$$

### Example 5: Experimental Testing of the Microsphere Model for Human Primary Hepatocytes

Cells: Donor information: Male, Caucasian, 36 years old (Lot No. NFX, BioIVT)

### Instruments:

| Instruments | Brand | Cat. No./Model |
|---|---|---|
| 96-well ultra-low | Corning | 4515 |
| adsorption culture plate | | |
| Centrifuge | Eppendorf | 5804R |
| Cell incubator | Thermo Scientific | 371 |
| Cell counter | Invitrogen | Countess II |
| Oscillator | IKA | MS 3 |
| Microplate reader | Tecan | Infinite M200 |

### Cell Inoculation and Culture:

1) One tube of cryopreserved hepatocytes was taken, ensuring the hepatocytes were cryogenically frozen until recovery. The hepatocytes were quickly placed in a 37° C. water bath and gently shaken until all ice crystals were completely dissolved. After spraying with 70% ethanol, the tube was transferred to a biosafety cabinet.
2) Transfer the contents of the hepatocyte tube into a 50 mL centrifuge tube containing 50 mL of recovery medium and centrifuge at 100 g for 3 min. After centrifugation, the recovery medium was aspirated, and sufficient incubation medium was added to obtain a cell suspension with a cell density of approximately 1.0×10⁶ cells/mL.
3) Count hepatocytes and determine viable cell density with Cellometer Vision. Make sure that the viability of hepatocytes is greater than 80%. Adjust the cell density to 4000 cells/well with inoculation medium, and inoculate 100 μL per well.
4) Incubated in a cell incubator and incubated at 37° C. for 7-9 days until microspheres were formed.

### Compound Preparation and Administration:

1) Dilute the compound with the cell culture medium according to the three-fold dilution method, and the dilution gradient is shown in the table below. The maximum concentration of the control compound is 200 M, the maximum concentration of the test compound (according to customer requirement) is 5 M and the final concentration of DMSO is 0.1%.
2) Perform administration after the microspheres are formed. Aspirate the medium in the cell culture plate wells and add 100 L of the compound working solution before each administration. Perform administration every three days until the fourteenth day after microsphere formation.

### Test Indicator Detection:

1) After 14 days of culture, remove the cell culture plates from the incubator and transfer 80 μL of supernatant to determine the albumin and lactate dehydrogenase content.
2) Thaw CellTiter-Fluor™ Cell Viability Kit at ambient temperature, add 10 μL of GF-AFC substrate to 10 mL of 2× working solution, and dilute into 1× working solution with an equal volume of PBS.
3) Working solution (100 μL) was added to each well of the cell culture plate and incubated at 37° C. for 30 min, then pipetted 80 μL of supernatant into a 96-well black plate and detected the absorbance at an excitation wavelength of 400 nm and an emission wavelength of 505 nm.

### Data Analysis:

1) The cell viability (% Vehicle) is calculated using the following formula:

$$\% Vehicle = \frac{{Read}_{Compound} - {Read}_{Blank}}{{Read}_{vehicle} - {Read}_{Blank}} \times 100 \%$$

2) The IC₅₀ is calculated using GraphPad Prism 8.0.2 with the following software calculation formula:

$$Y = Bottom + ( Top - Bottom ) / ( 1 + 10 \hat{} ( ( Log IC 50 - X ) * HillSlope ) )$$

What is claimed is:

1. (canceled)

2. A compound having a structure of formula (II), or pharmaceutically acceptable salts, stereoisomers, isotope isomers, prodrugs, hydrates, or solvates of the compound: 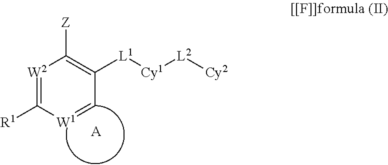
wherein, W¹ represents C;
wherein, W² represents CH or N;
wherein, Z represents 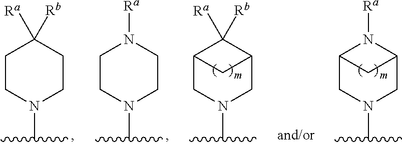 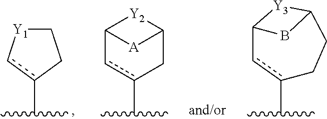 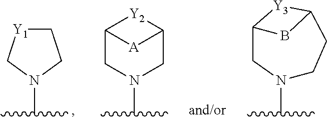
wherein L¹ represents —C(O)NH—, —HNC(O)—, or 5-6 membered heteroaryl 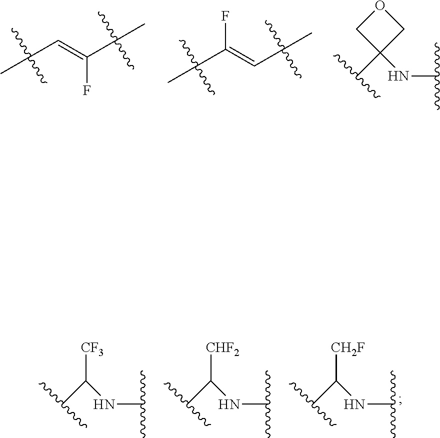
wherein, L² represents absence or —C₁-C₆ alkylene;
wherein, R¹ represents L³-R³;
wherein, L³ represents —NR^{a}SO₂—;
wherein, R³ represents C₁-C₆ alkyl, wherein the C₁-C₆ alkyl is optionally independently substituted with 0-3 substituents selected from halogen and —OR^{a};
wherein, Cy¹ represents 6-12-membered aryl or 5-12-membered heteroaryl; the Cy¹ is optionally substituted with 0-3 substituents selected from the following: halogen, C₁-C₆ alkyl, C₁-C₆ haloalkyl, hydroxy(C₁-C₆ alkyl), OR^{a}, —O—(C₁-C₆ haloalkyl), 5-6-membered heteroaryl, phenyl, —SR^{a}, —SF₅, cyano, nitro, —NR^{a}R^{b}, —NR^{a}C(O)R^{b}, —C(O)NR^{a}R^{b}, —OC(O)R^{a}, —C(O)R^{a}, —P(O)R^{a}R, —C(O)OR^{a}, —S(O)R^{a}, —S(O)₂R^{a}, and —S(O)₂NR^{a}R^{b};
wherein, Cy² represents a 3-12-membered saturated or unsaturated monocyclic or bicyclic ring, the 3-12-membered saturated or unsaturated monocyclic or bicyclic ring optionally contains 0-3 heteroatoms selected from O, N, and S; the Cy² is optionally substituted with 0-3 substituents selected from the following: halogen, C₁-C₆ haloalkyl, C₁-C₆ haloalkyl, hydroxy (C₁-C₆ alkyl), —OR^{a}, —O—(C₁-C₆ haloalkyl), —SR, —SF₅, cyano, nitro, —NR^{a}R^{b}, —NR^{a}C(O)R^{b}, —C(O)NR^{a}R^{b}, —OC(O)R^{a}, —C(O)OR^{a}, —S(O)R^{a}, —S(O)₂R^{a}, and —S(O)₂NR^{a}R;
wherein, ring A represents a saturated or unsaturated 4-12-membered ring, preferably a 5-12-membered ring; the saturated or unsaturated 4-12-membered ring optionally contains 0-3 heteroatoms selected from O, N, and S; the ring A is optionally substituted with 0-3 substituents selected from the following: halogen, C₁-C₆ alkyl, C₁-C₆ haloalkyl, hydroxy C₁-C₆ alkyl, —OR^{a}, —O—C₁-C₆ haloalkyl, —SR^{a}, —SF₅, cyano, nitro, —NR^{a}R^{b}, —NR^{a}C(O)R^{b}, —C(O)NR^{a}R, —OC(O)R^{a}, —C(O)OR^{a}, —S(O)R^{a}, —S(O)₂R^{a}, and —S(O)₂NR^{a}R^{b};
wherein, R^{a}, R^{b} each independently represent hydrogen, halogen, C₁-C₆ alkyl, C₃-C₆ cycloalkyl, C₁-C₆ haloalkyl, or hydroxy (C₁-C₆ alkyl); or R^{a} and R^{b}, together with an atom attaching to the R^{a} and the R^{b}, form a 3-6-membered saturated or unsaturated ring wherein the 3-6-membered saturated or unsaturated ring optionally contains 0-2 heteroatoms selected from O, S, and N; wherein, m represents integers from 0 to 3.

3. (canceled)

4. (canceled)

5. (canceled)

6. (canceled)

7. (canceled)

8. (canceled)

9. (canceled)

10. (canceled)

11. The compound according to claim 2, or the pharmaceutically acceptable salts, the stereoisomers, the isotope isomers, the prodrugs, the hydrates, or the solvates of the compound, wherein, Z represents: 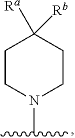
wherein, R^{a} and R^{b} each independently represent hydrogen, C₁-C₆ alkyl, C₃-C₆ cycloalkyl, C₁-C₆ haloalkyl or hydroxy(C₁-C₆ alkyl); or R^{a} and R^{b}, together with the atom attaching to the R^{a} and the R^{b}, form the 3-6-membered saturated or unsaturated ring wherein the 3-6-membered saturated or unsaturated ring optionally contains 0-2 heteroatoms selected from O, S, and N.

12. The compound according to claim 2, or the pharmaceutically acceptable salts, the stereoisomers, the isotope isomers, the prodrugs, the hydrates, or the solvates of the compound, wherein Z represents:

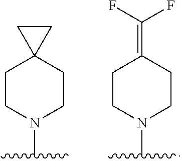 [SMILES: *N1CCC2(CC1)CC2 ; *N1CCC(=C(F)F)CC1]

13. The compound according to claim 2, or the pharmaceutically acceptable salts, the stereoisomers, the isotope isomers, the prodrugs, the hydrates, or the solvates of the compound, wherein L¹ represents: —C(O)NH—

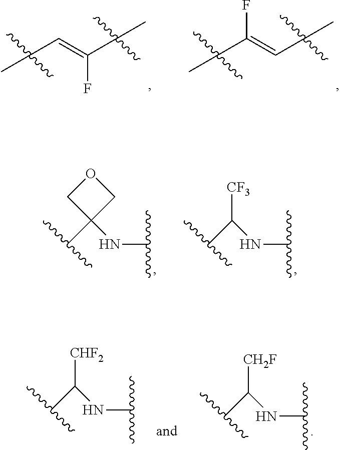

14. The compound according to claim 2, or the pharmaceutically acceptable salts, the stereoisomers, the isotope isomers, the prodrugs, the hydrates, or the solvates of the compound, wherein L¹ represents 5-6-membered heteroaryl.

15. (canceled)

16. The compound according to claim 14, or the pharmaceutically acceptable salts, the stereoisomers, the isotope isomers, the prodrugs, the hydrates, or the solvates of the compound, wherein L¹ represents one of the following groups:

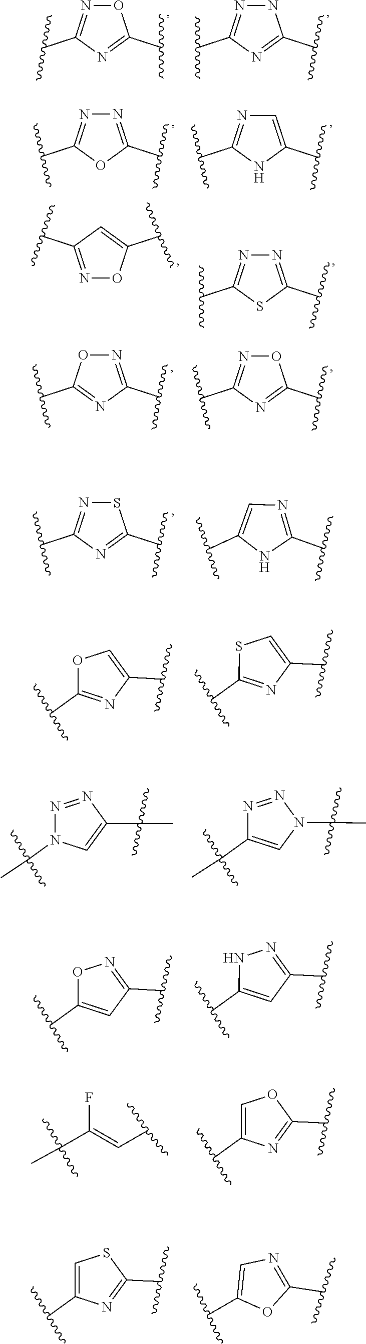

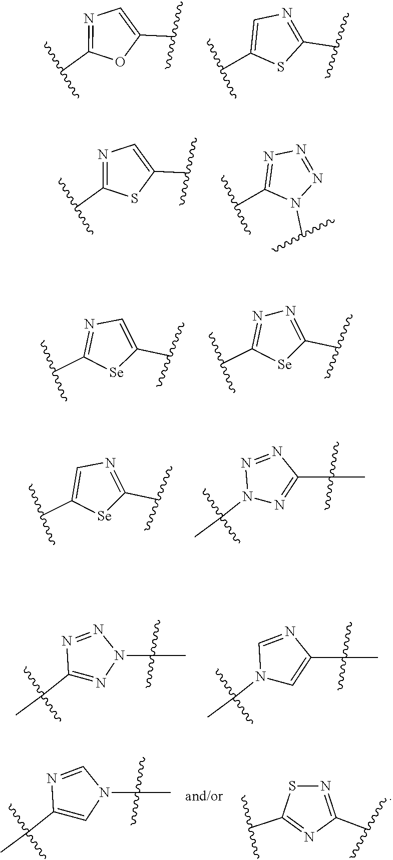

17. The compound according to claim 2, or the pharmaceutically acceptable salts, the stereoisomers, the isotope isomers, the prodrugs, the hydrates, or the solvates of the compound, wherein, Cy¹ represents one of the following groups substituted with 0-3 substituents selected from halogen, C₁-C₆ alkyl, C₁-C₆ haloalkyl, hydroxy(C₁-C₆ alkyl), OR^{a}, —O—C₁-C₆ haloalkyl, 5-6-membered heteroaryl, phenyl, —SR, —SF₅, cyano, nitro, —NR^{a}R^{b}, —NR^{a}C(O)R^{b}, —C(O)NR^{a}R^{b}, —OC(O)R^{a}—C(O)R^{a}, —P(O)R^{a}R^{b}, —C(O)OR^{a}, —S(O)R a —S(O)R^{a} and —S(O)₂NR^{a}R^{b} :

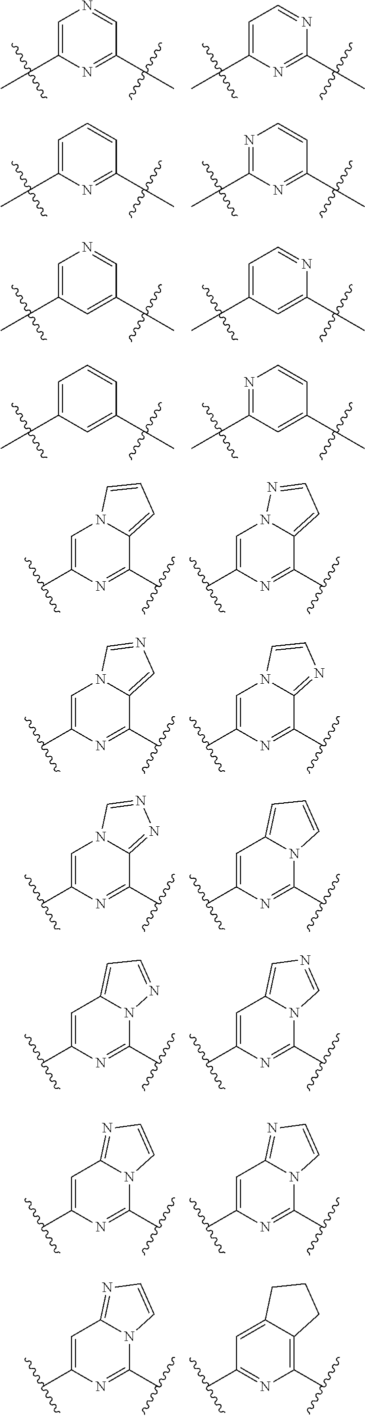

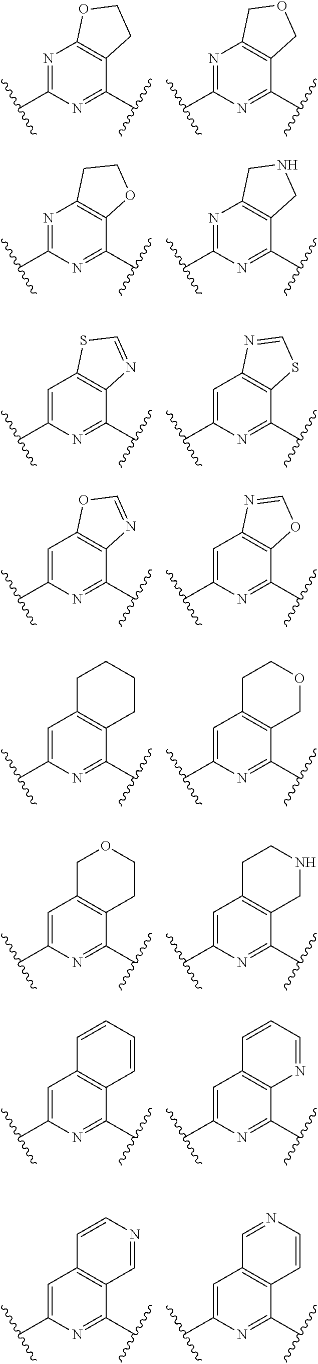

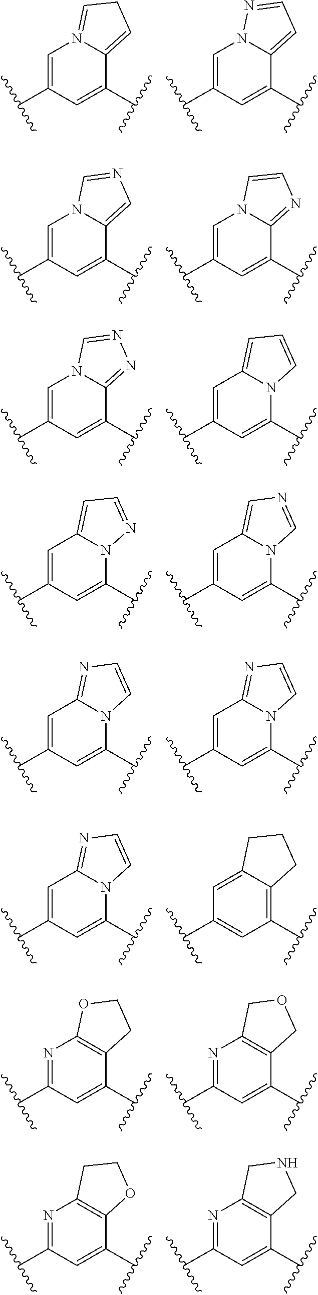

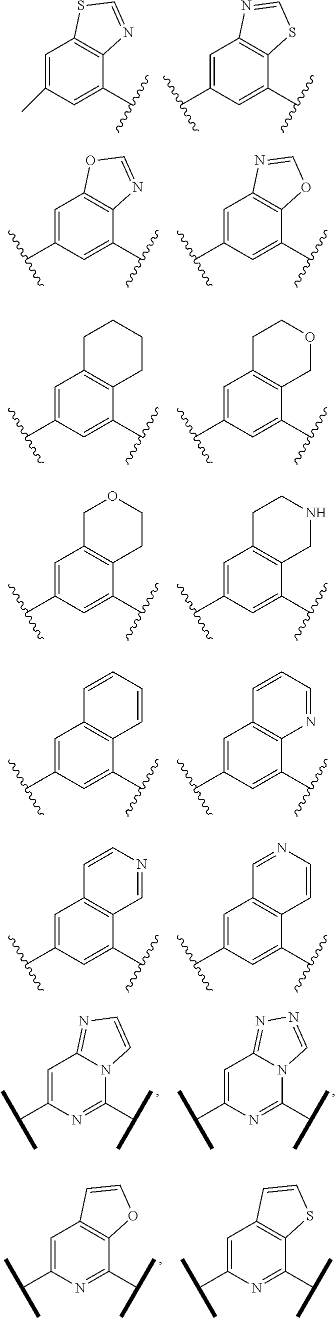

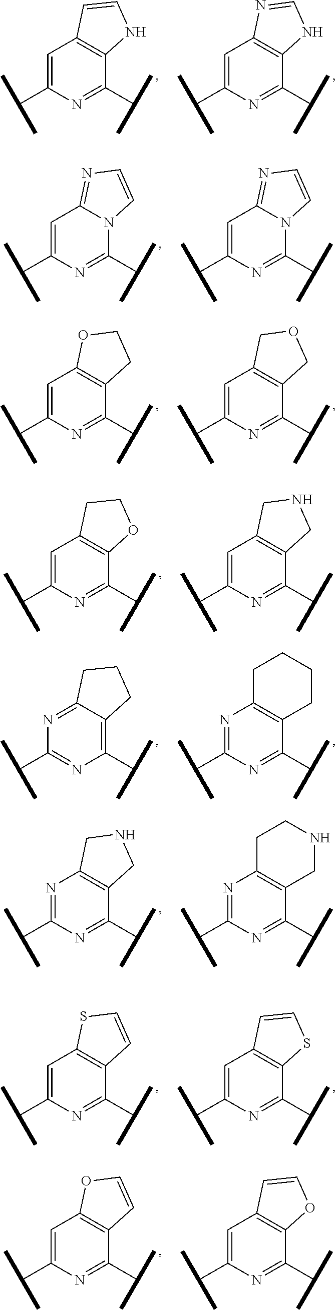

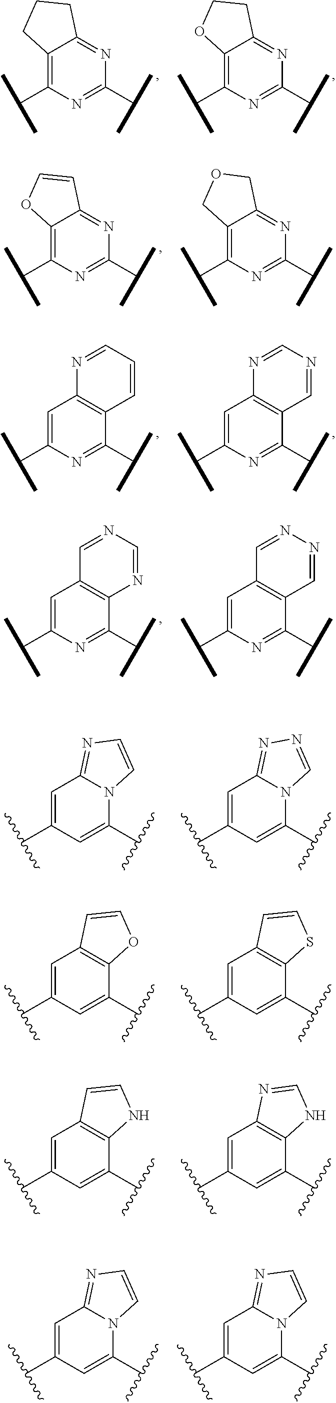

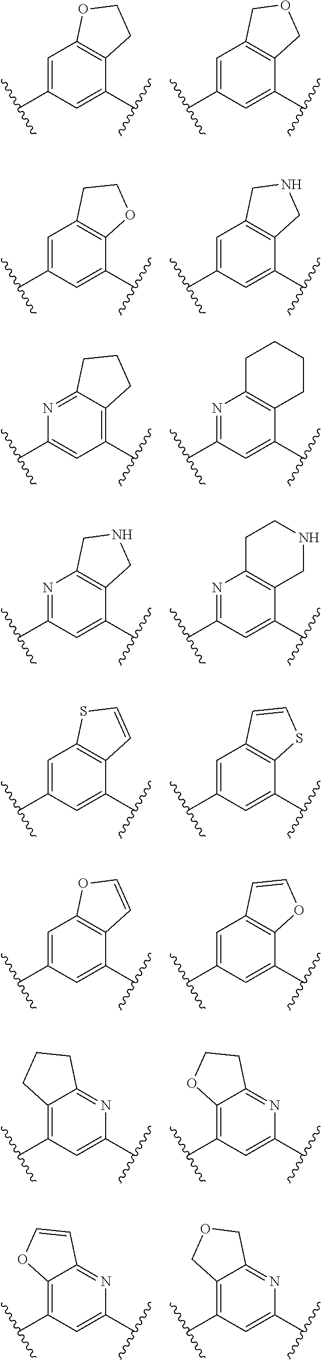

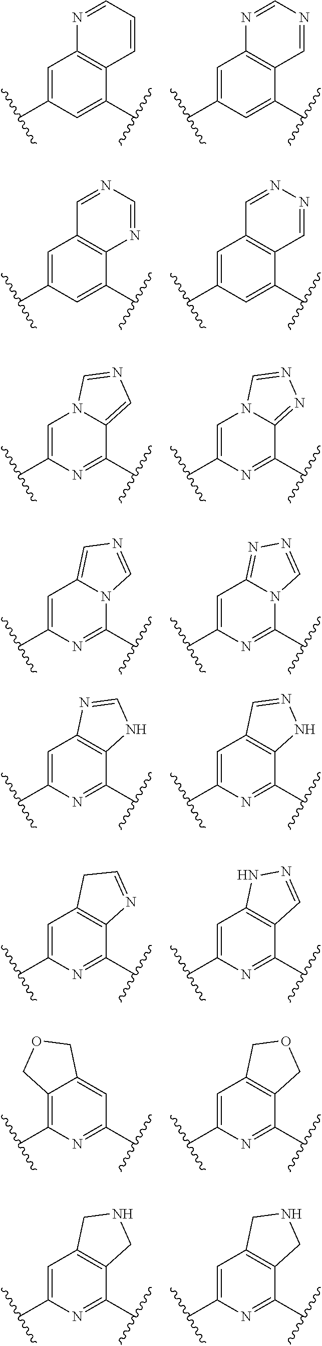

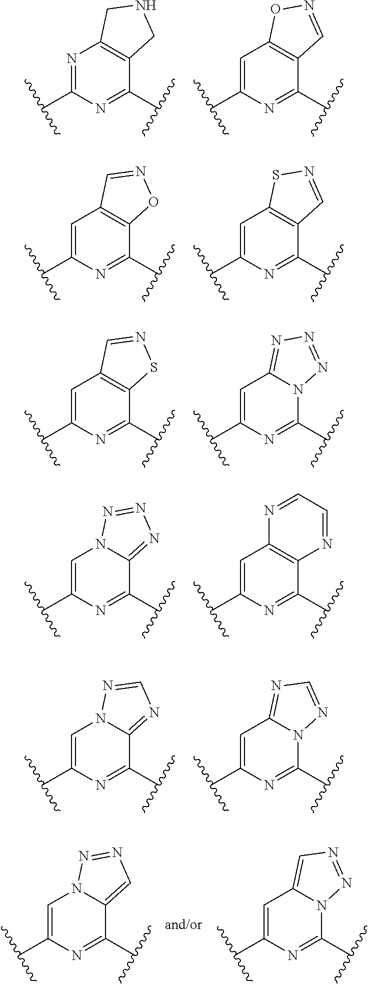

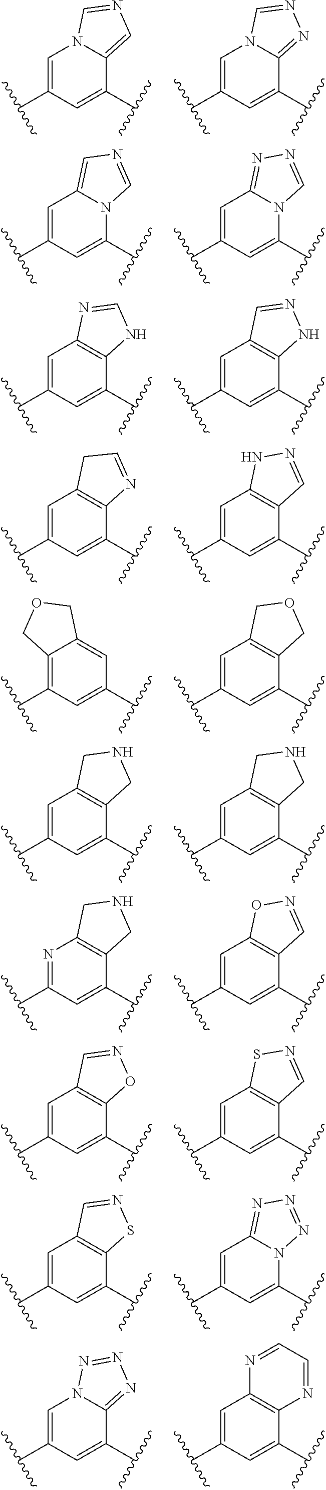

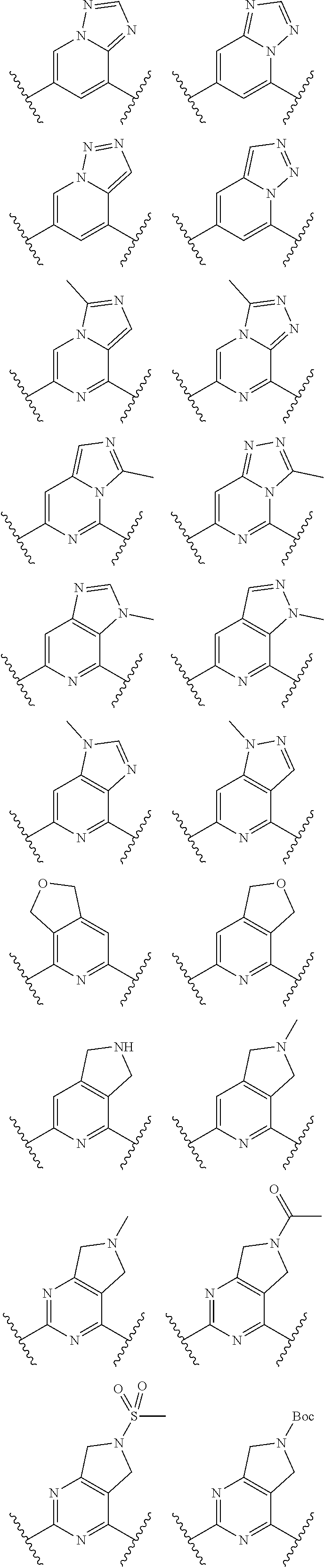

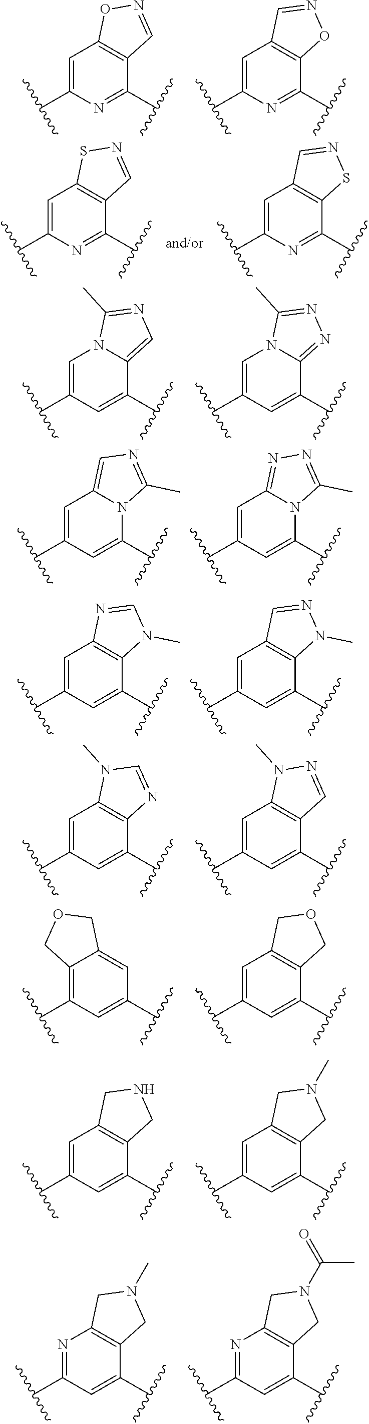

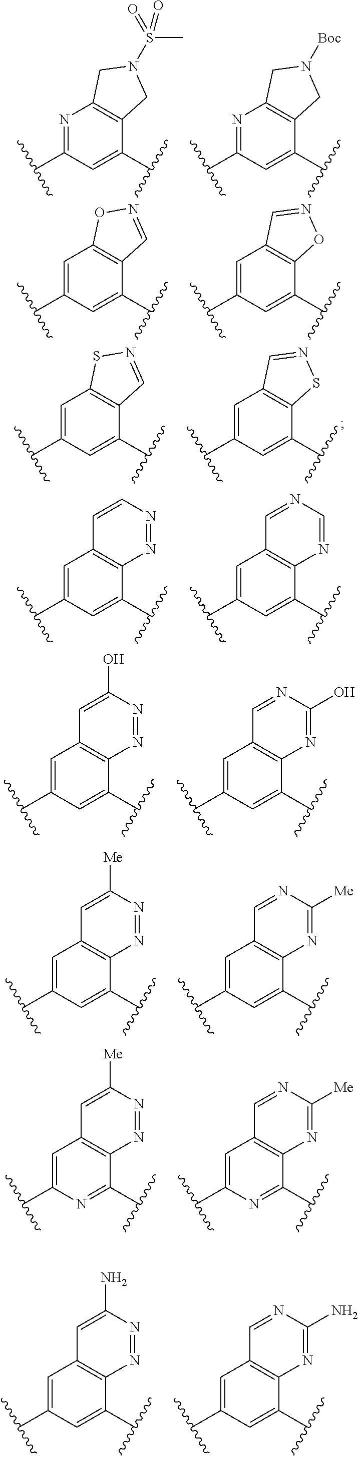

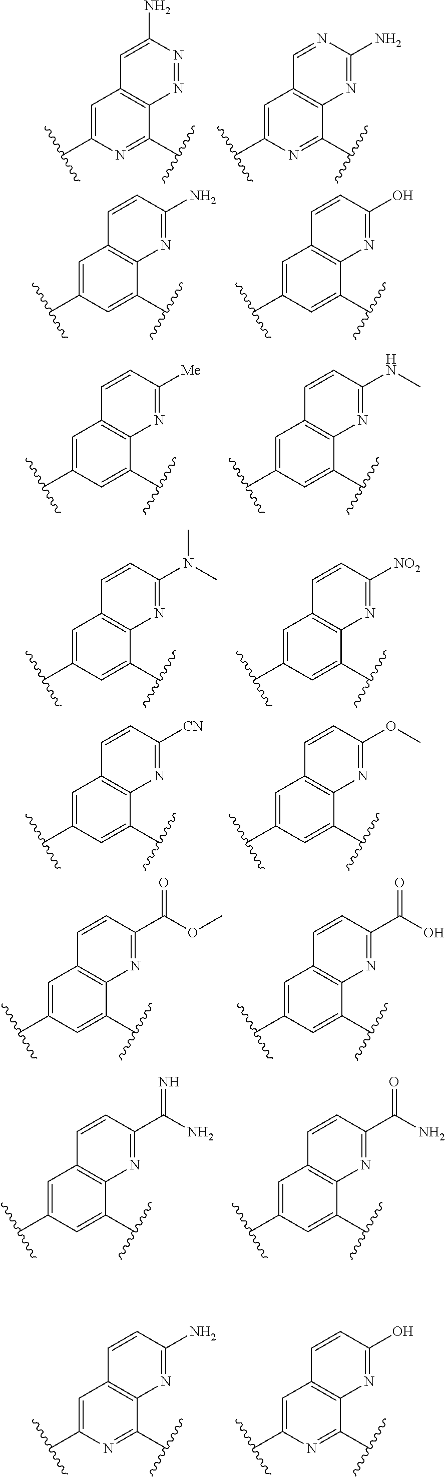

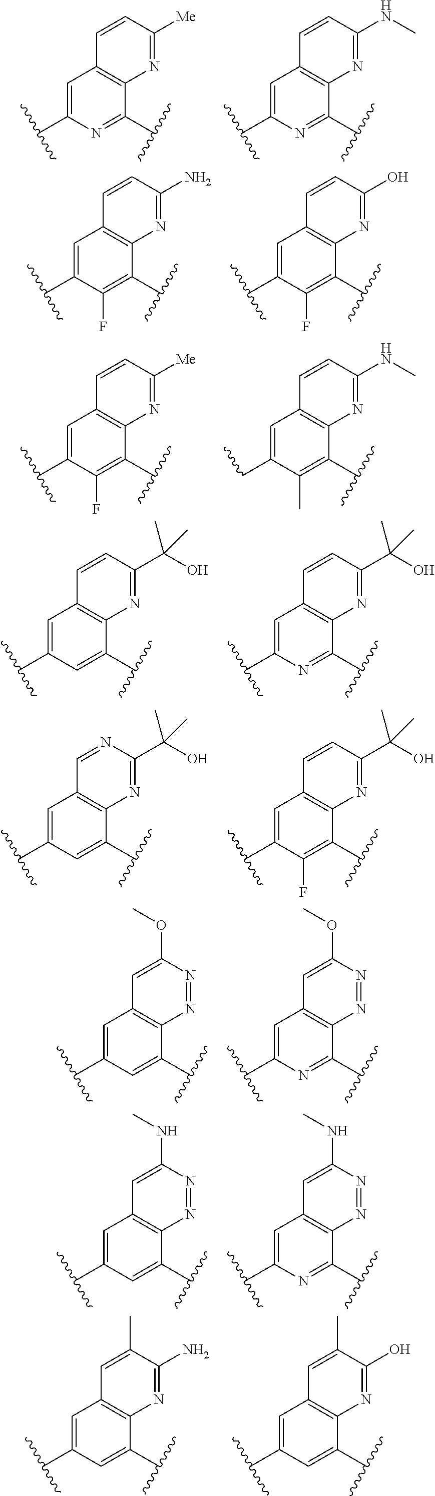

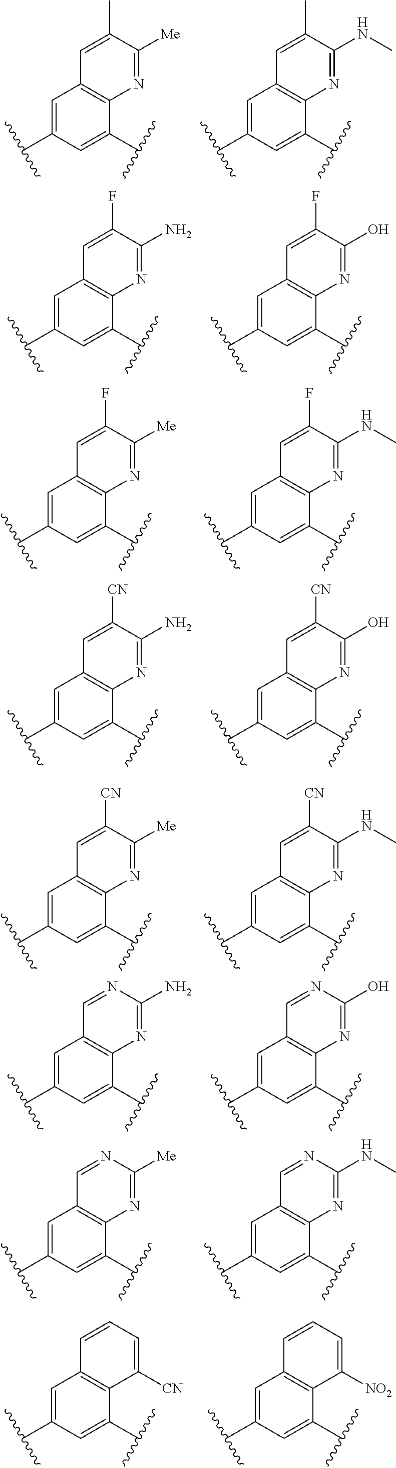

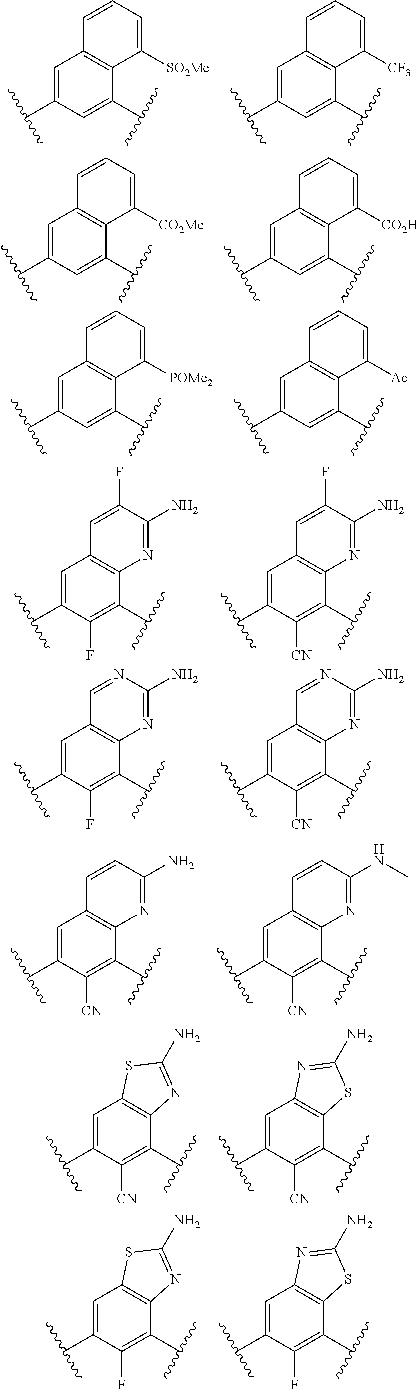

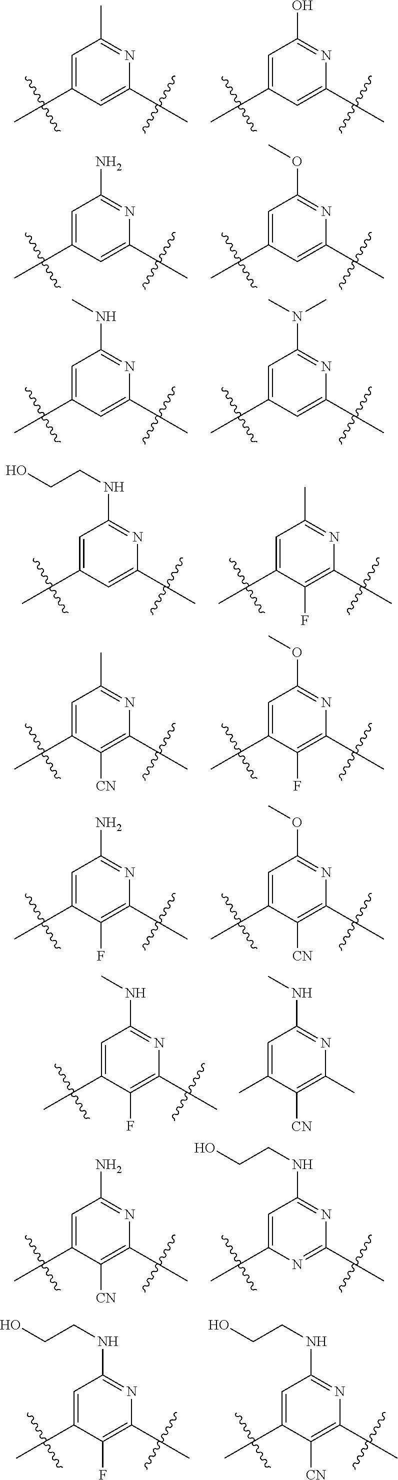

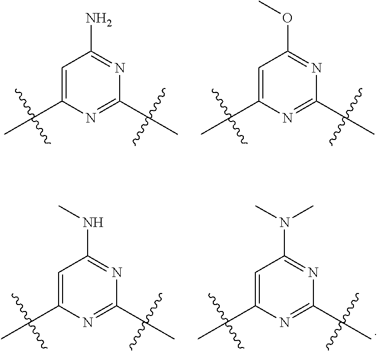

18. The compound according to claim 17, or the pharmaceutically acceptable salts, the stereoisomers, the isotope isomers, the prodrugs, the hydrates, or the solvates of the compound, wherein, Cy¹ represents one of the following groups substituted with 0-3 substituents selected from halogen, C₁-C₆ alkyl, C₁-C₆ haloalkyl, hydroxy C₁-C₆ alkyl, OR^{a}, —O—C₁-C₆ haloalkyl, 5-6 membered heteroaryl, phenyl, —SR, —SF₅, cyano, nitro, —NR^{a}R^{b}, —NR^{a}C(O)R^{b}, —C(O)NR^{a}R^{b}, —OC(O)R^{a}, —C(O)R^{a}, —P(O)R^{a}R^{b}, —C(O)OR^{a}, —S(O)R^{a}, —S(O)₂R^{a}, and —S(O)₂NR^{a}R^{b}:

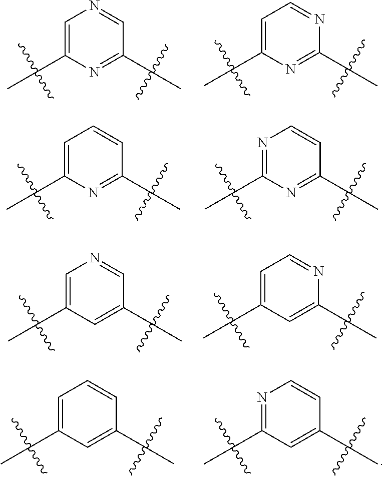

19. The compound according to claim 18, or the pharmaceutically acceptable salts, the stereoisomers, the isotope isomers, the prodrugs, the hydrates, or the solvates of the compound, wherein, Cy¹ represents 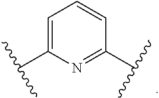 [SMILES: *c1cccc(*)n1]
substituted with 0-3 substituents selected from halogen, C₁-C₆ alkyl, C₁-C₆ haloalkyl, hydroxy C₁-C₆ alkyl, OR^{a}, —O—C₁-C₆ haloalkyl, 5-6-membered heteroaryl, phenyl, —SR, —SF₅, cyano, nitro, —NR^{a}R^{b}, —NR^{a}C(O)R^{b}, —C(O)NR^{a}R^{b}, —OC(O)R^{a}, —C(O)R^{a}, —P(O)R^{a}R, —C(O)OR^{a}, —S(O)R^{a}, —S(O)₂R^{a}, and —S(O)₂NR^{a}R^{b}.

20. The compound according to claim 18, or the pharmaceutically acceptable salts, the stereoisomers, the isotope isomers, the prodrugs, the hydrates, or the solvates of the compound, wherein Cy¹ represents 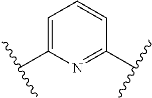 [SMILES: *c1cccc(*)n1]
substituted with phenyl, pyridinyl, thiazolyl, oxazolyl, pyrazolyl, imidazolyl, and N-methylpyrazolyl.

21. The compound according to claim 18, or the pharmaceutically acceptable salts, the stereoisomers, the isotope isomers, the prodrugs, the hydrates, or the solvates of the compound, wherein Cy¹ represents the following groups:

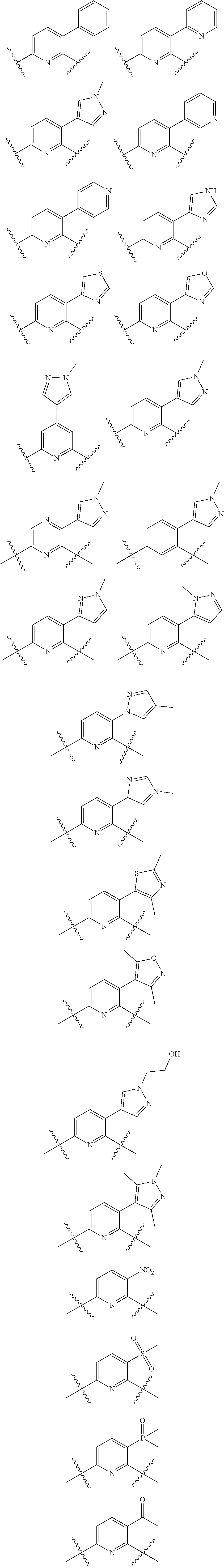

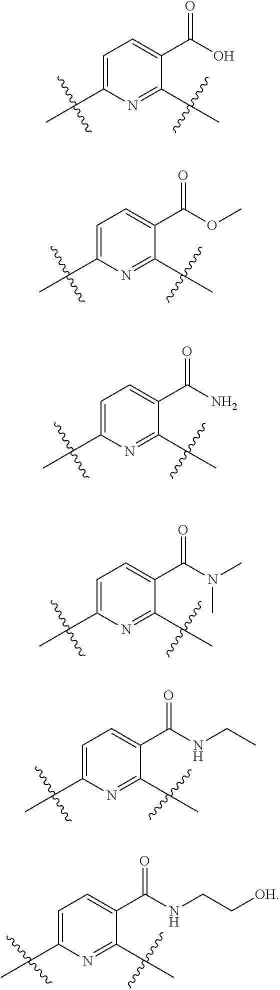 [SMILES: *c1ccc(C(=O)O)c(*)n1 ; *c1ccc(C(=O)OC)c(*)n1 ; *c1ccc(C(N)=O)c(*)n1 ; *c1ccc(C(=O)N(C)C)c(*)n1 ; *c1ccc(C(=O)NCC)c(*)n1 ; *c1ccc(C(=O)NCCO)c(*)n1]

22. The compound according to claim 18, or the pharmaceutically acceptable salts, the stereoisomers, the isotope isomers, the prodrugs, the hydrates, or the solvates of the compound, wherein, Cy¹ represents 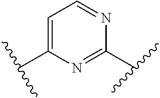 [SMILES: *c1ccnc(*)n1]
substituted with 0-3 substituents selected from halogen, C₁-C₆ alkyl, C₁-C₆ haloalkyl, hydroxy C₁-C₆ alkyl, OR^{a}, —O—C₁-C₆ haloalkyl, 5-6-membered heteroaryl, phenyl, —SR, —SF₅, cyano, nitro, —NR^{a}R^{b}, —NR^{a}C(O)R^{b}, —C(O)NR^{a}R^{b}, —OC(O)R^{a}, —C(O)R^{a}, —P(O)R^{a}R^{b}, —C(O)OR^{a}, —S(O)R^{a}, —S(O)₂R^{a}, and —S(O)₂NR^{a}R^{b}.

23. (canceled)

24. The compound according to claim 2, or the pharmaceutically acceptable salts, the stereoisomers, the isotope isomers, the prodrugs, the hydrates, or the solvates of the compound, wherein L² represents absence.

25. The compound according to claim 2, or the pharmaceutically acceptable salts, the stereoisomers, the isotope isomers, the prodrugs, the hydrates, or the solvates of the compound, wherein Cy² is substituted with 0-3 substituents selected from the following: halogen, cyano, nitro, hydroxy C₁-C₆ alkyl, C₁-C₆ alkyl, C₃-C₅ cycloalkyl, C₁-C₆ haloalkyl, —OR^{a}, —SO₃R^{a}, —S(O)R^{a}, —OC₁-C₆ haloalkyl, —SR, —SF₅ or NR^{a}R-substituted morpholinyl, piperidinyl, azetidine, pyrrolidinyl, cyclopropyl, cyclobutyl, cyclopentyl, cyclohexyl, piperazinyl, or tetrahydrofuryl; wherein, R^{a} and R^{b} each independently represent hydrogen, halogen, C₁-C₆ alkyl, C₃-C₅ cycloalkyl, C₁-C₆ haloalkyl, or hydroxy C₁-C₆ alkyl; or R^{1a} and R^{1b}, together with an atom attaching to the R^{1a} and the R^{1b}, form a 3-6 membered ring, wherein the 3-6 membered ring contains 0-2 heteroatoms selected from O, N, and S.

26. The compound according to claim 14, or the pharmaceutically acceptable salts, the stereoisomers, the isotope isomers, the prodrugs, the hydrates, or the solvates of the compound, wherein Cy² represents

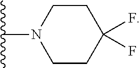 [SMILES: *N1CCC(F)(F)CC1]

27. (canceled)

28. (canceled)

29. (canceled)

30. The compound according to claim 2, or the pharmaceutically acceptable salts, the stereoisomers, the isotope isomers, the prodrugs, the hydrates, or the solvates of the compound, wherein the ring A, together with a fused ring 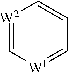
of the ring A, forms the following structures: 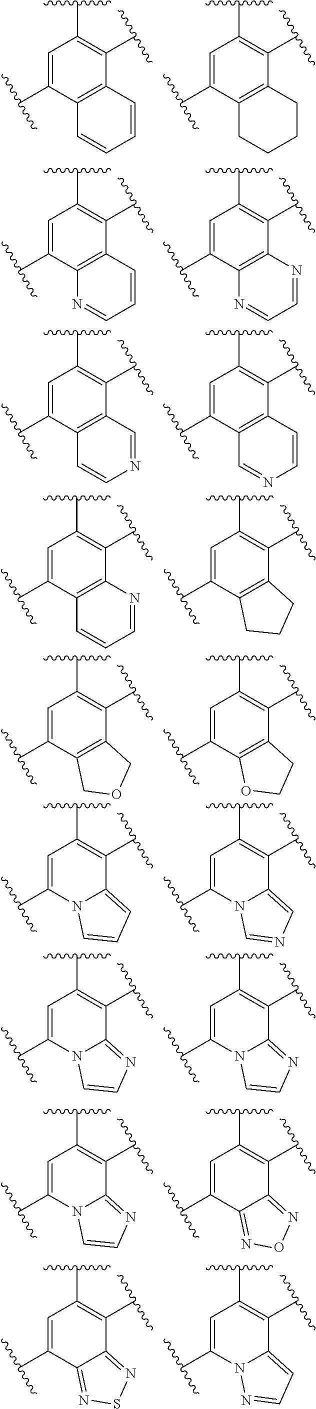 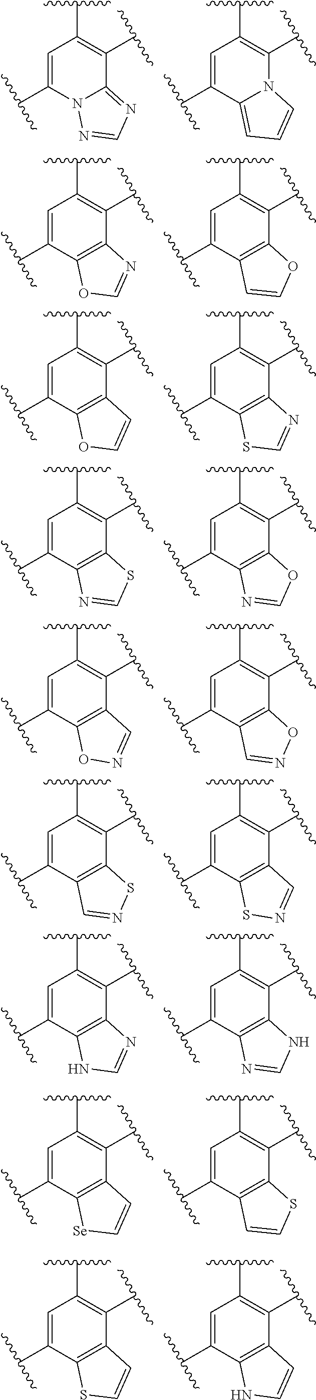 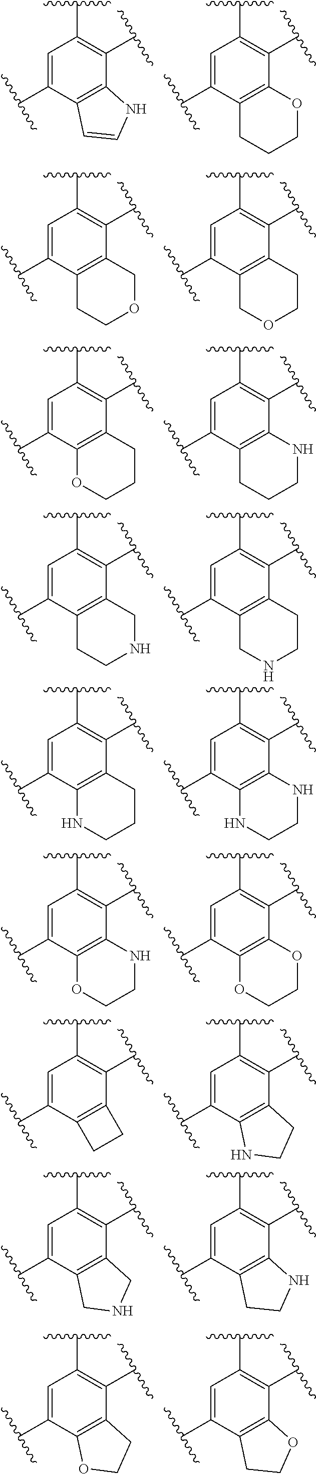 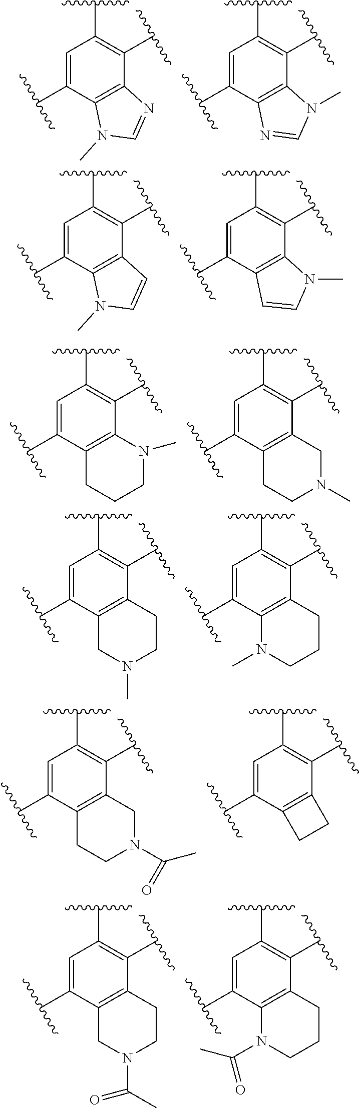
the ring A is optionally substituted with 0-3 substituents selected from the following: halogen, C₁-C₆ alkyl, C₁-C₆ haloalkyl, hydroxy C₁-C₆ alkyl, —OR^{a}, —O—C₁-C₆ haloalkyl, —SR^{a}, —SF₅, cyano, nitro, —NR^{a}R^{b}, —NR^{a}C(O)R^{b}, —C(O)NR^{a}R^{b}, —OC(O)R^{a}, —C(O)OR^{a}, —S(O)R^{a}, —S(O)₂R^{a}, and —S(O)₂NR^{a}R^{b}.

31. The compound according to claim 2, or the pharmaceutically acceptable salts, the stereoisomers, the isotope isomers, the prodrugs, the hydrates, or the solvates of the compound, wherein the ring A, together with a fused ring 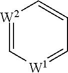
of the ring A, forms the following structures:

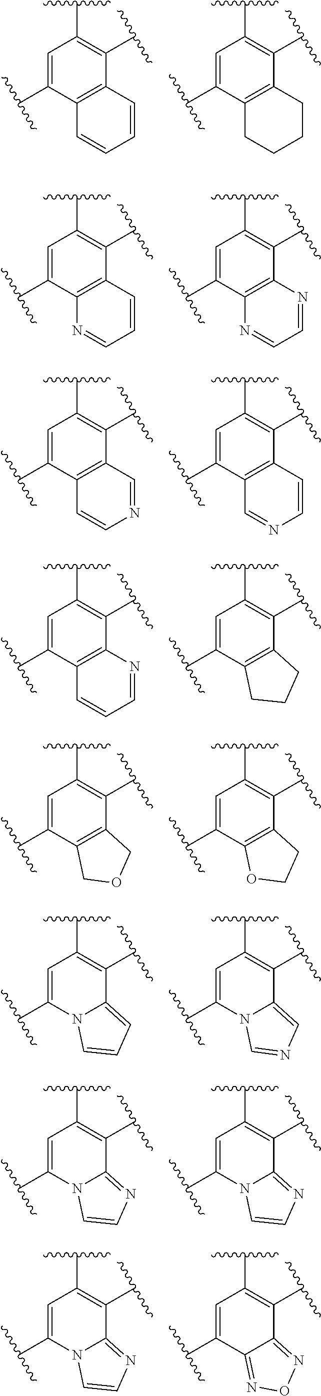

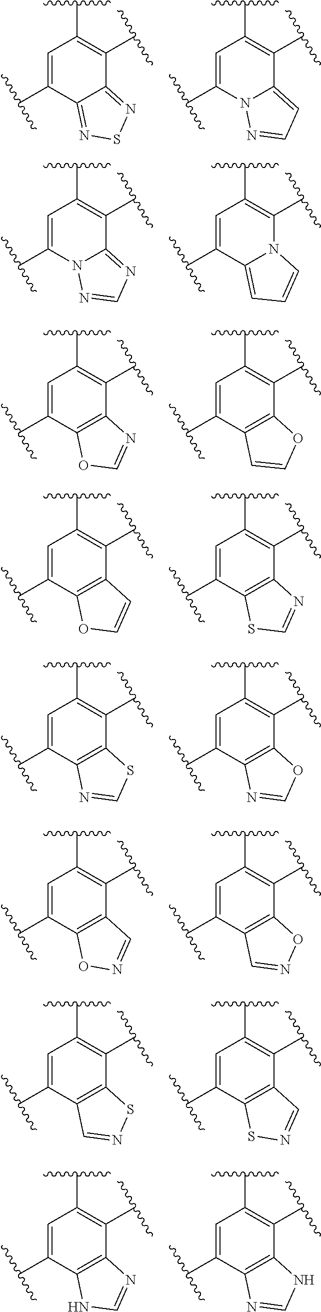

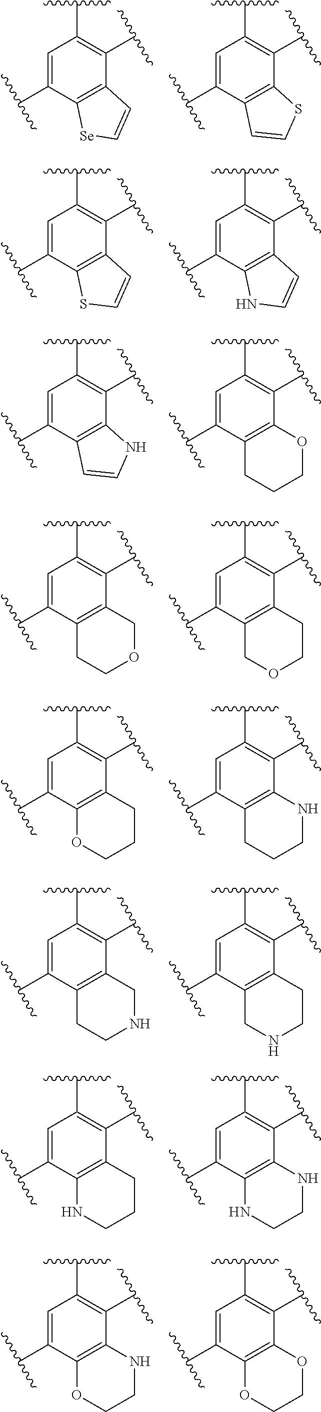

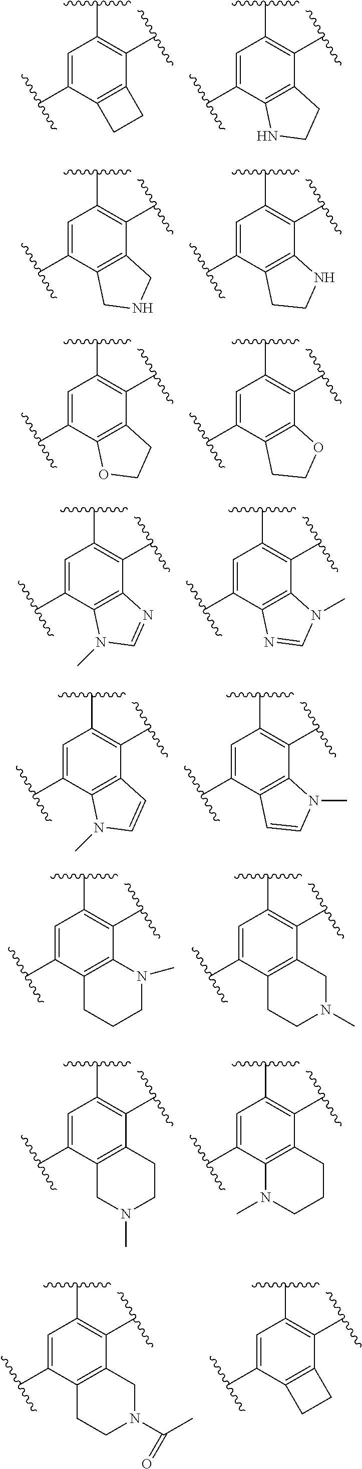

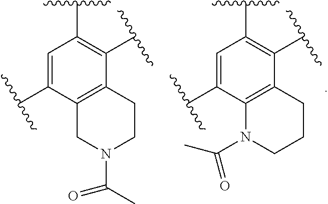 [SMILES: *c1cc(*)c2c(c1*)CCN(C(C)=O)C2 ; *c1cc(*)c2c(c1*)CCCN2C(C)=O]

32. Compounds with the following structures: 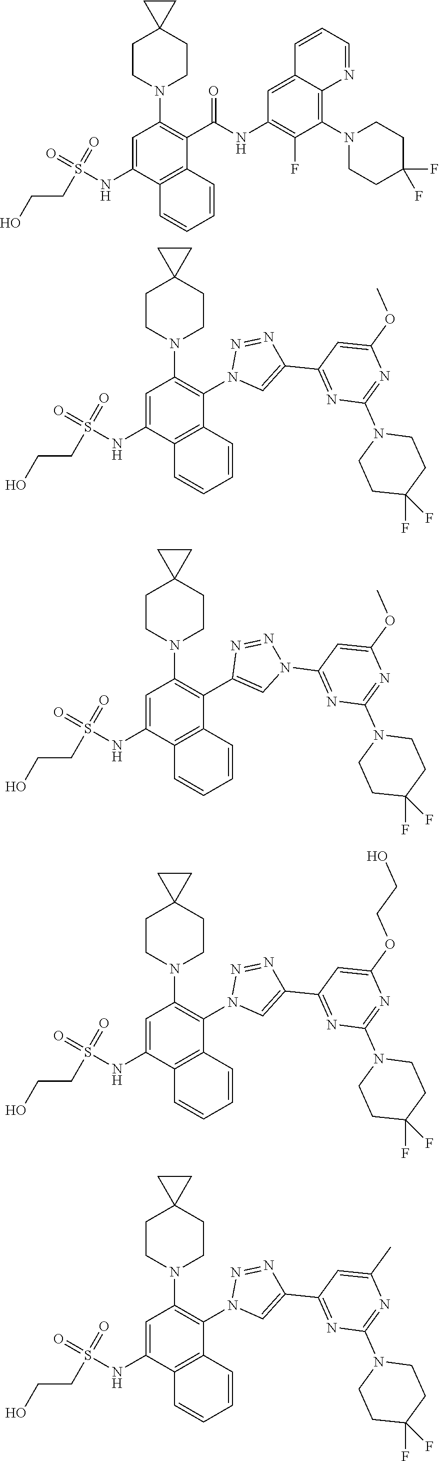 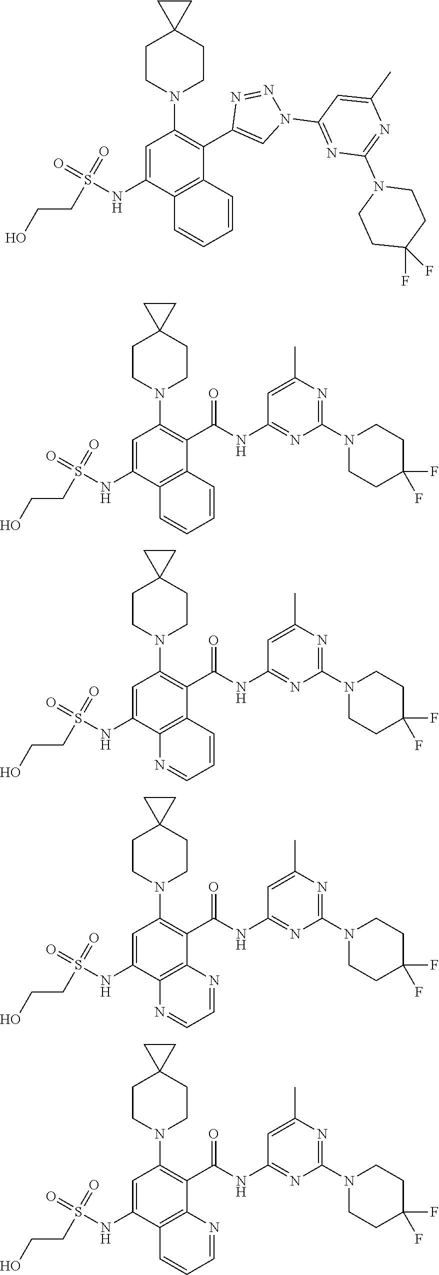 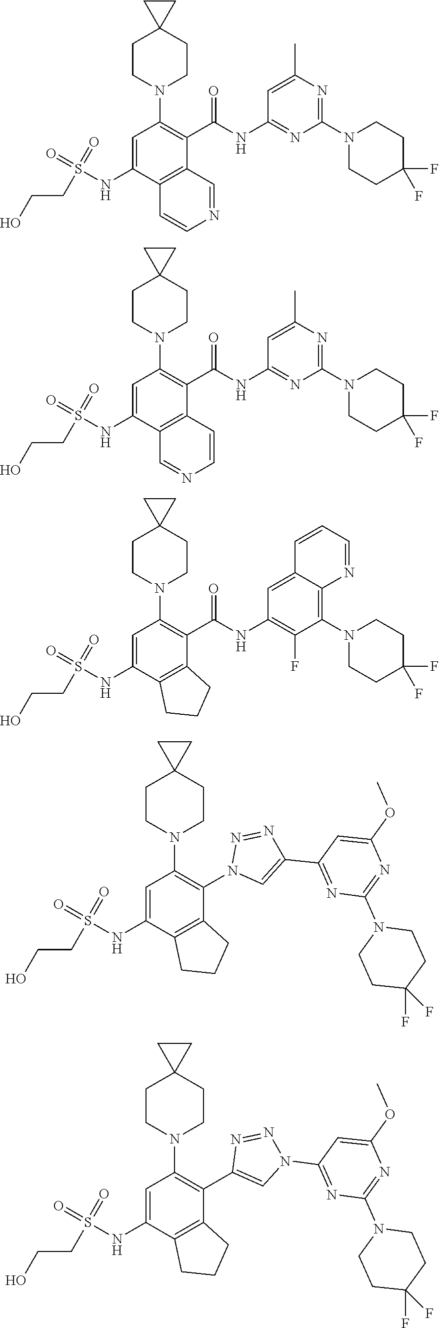 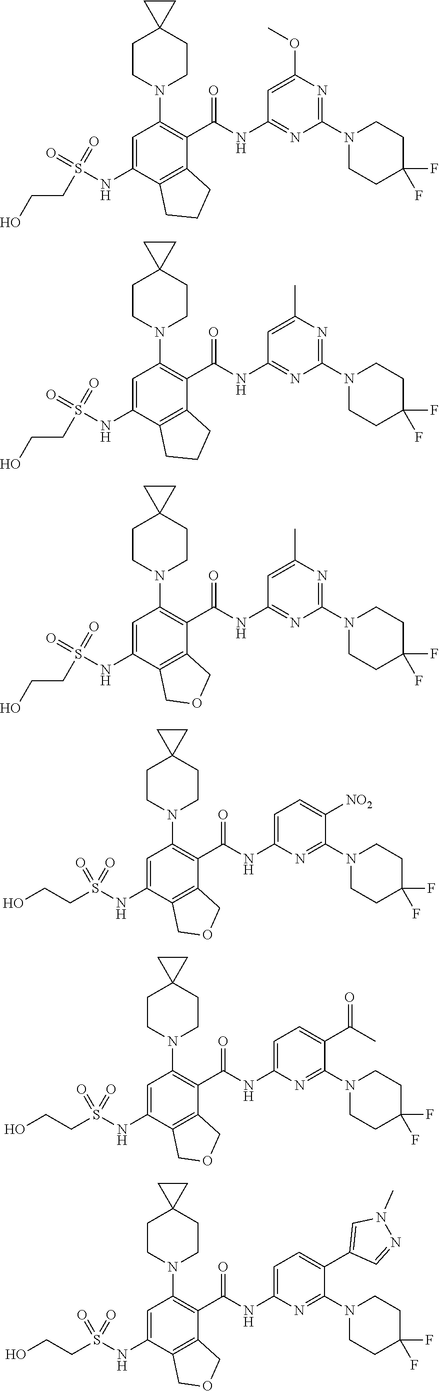 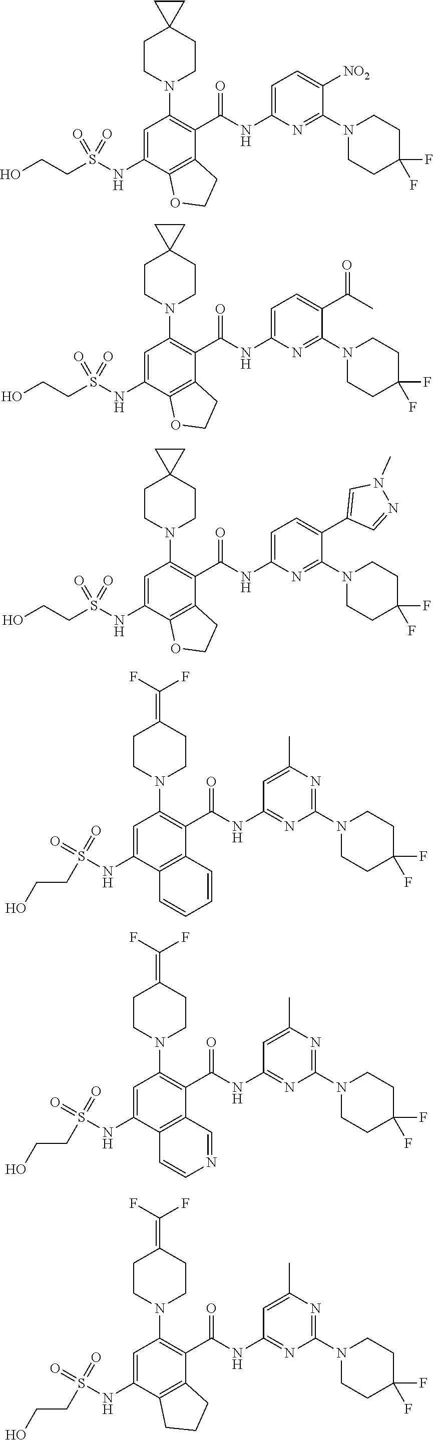 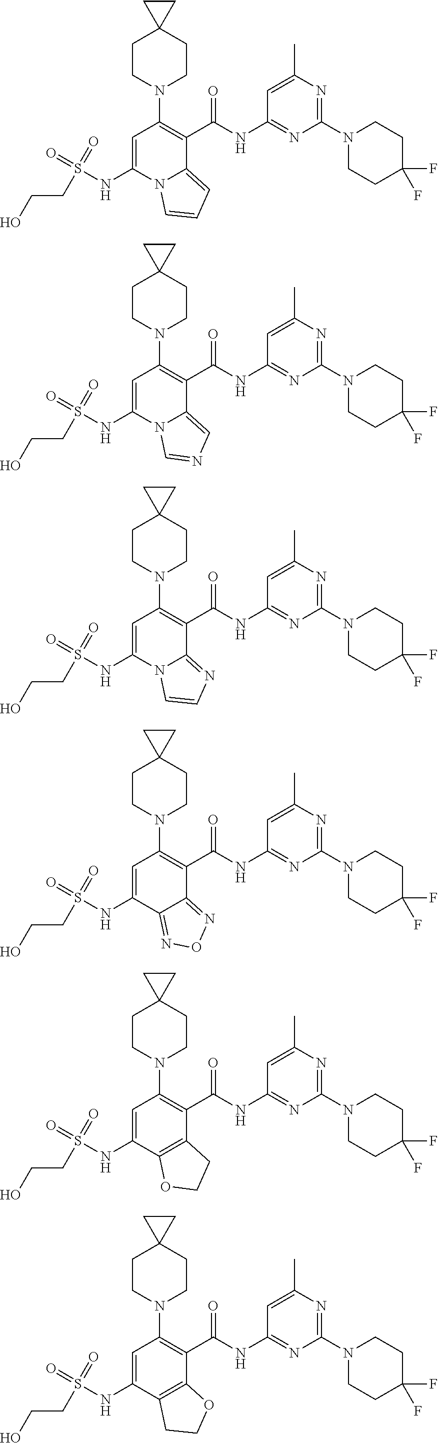 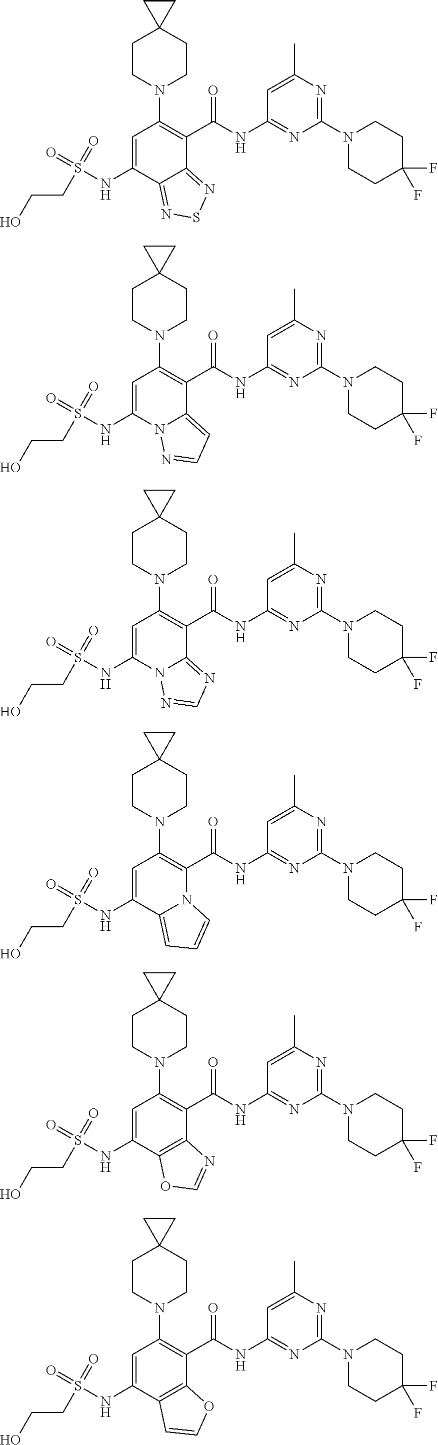 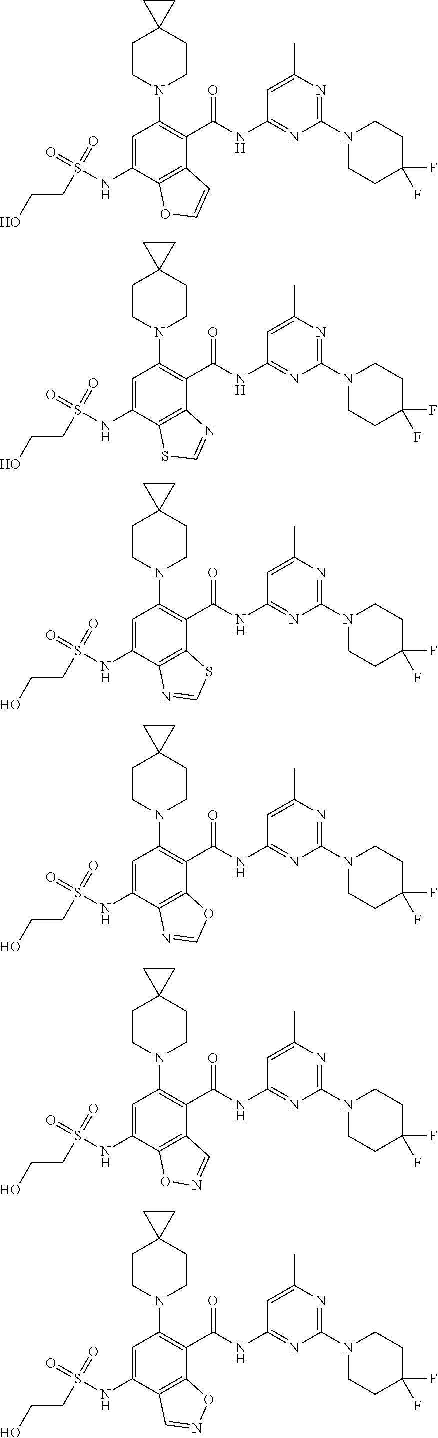 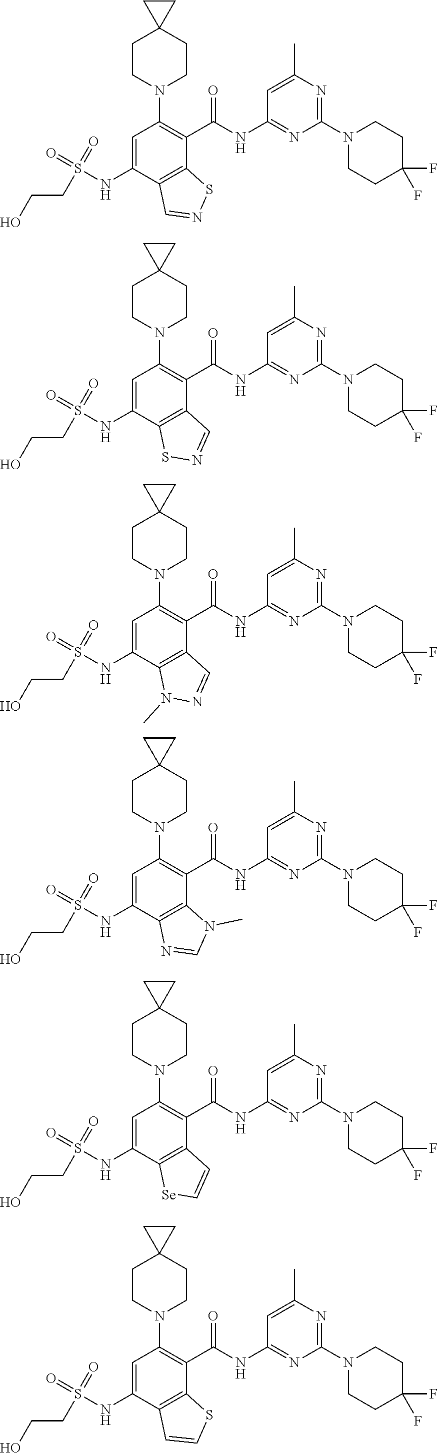 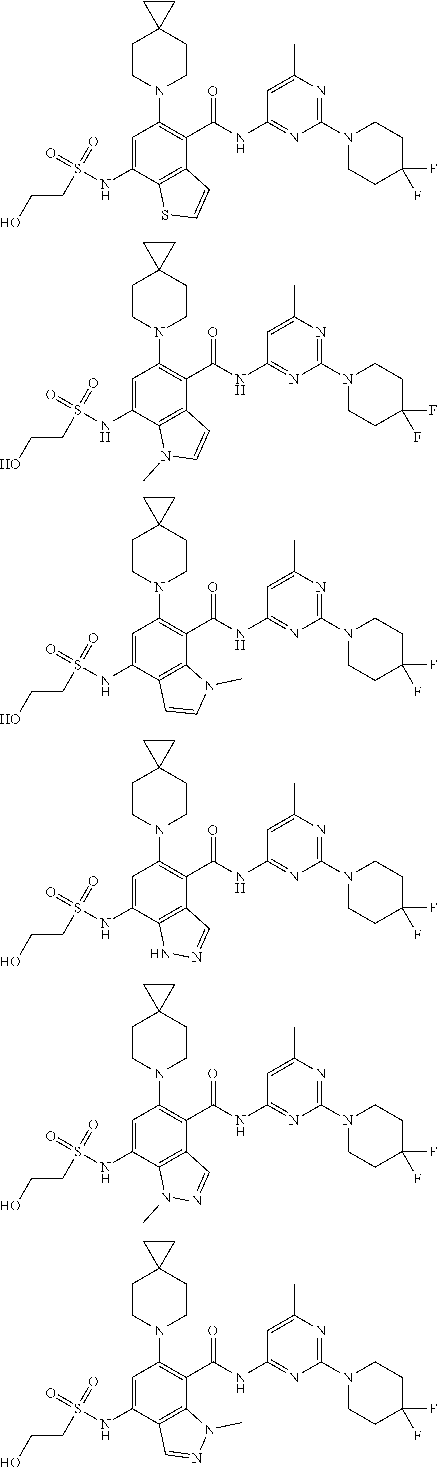 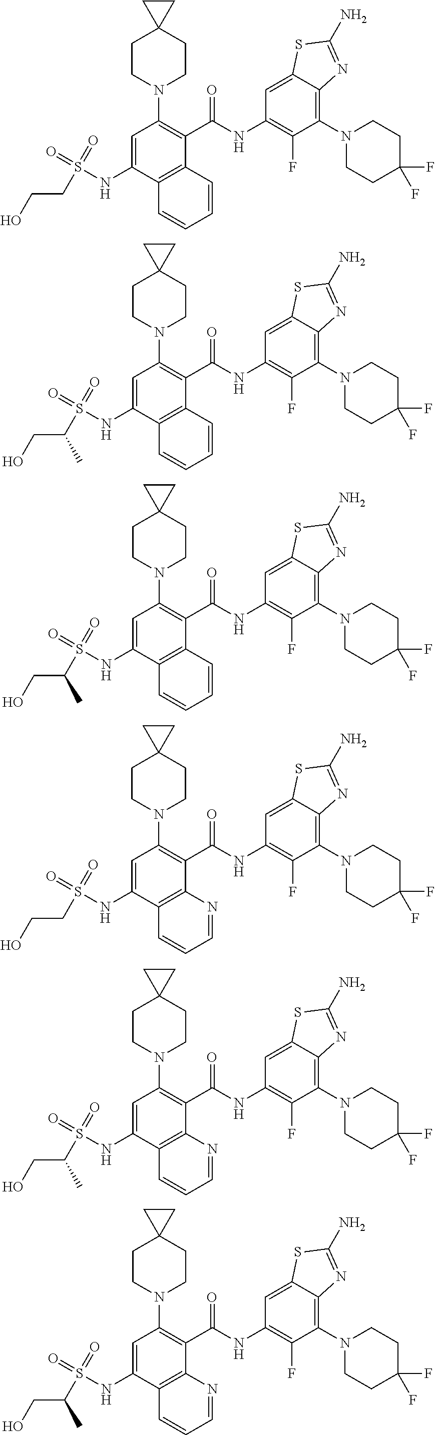 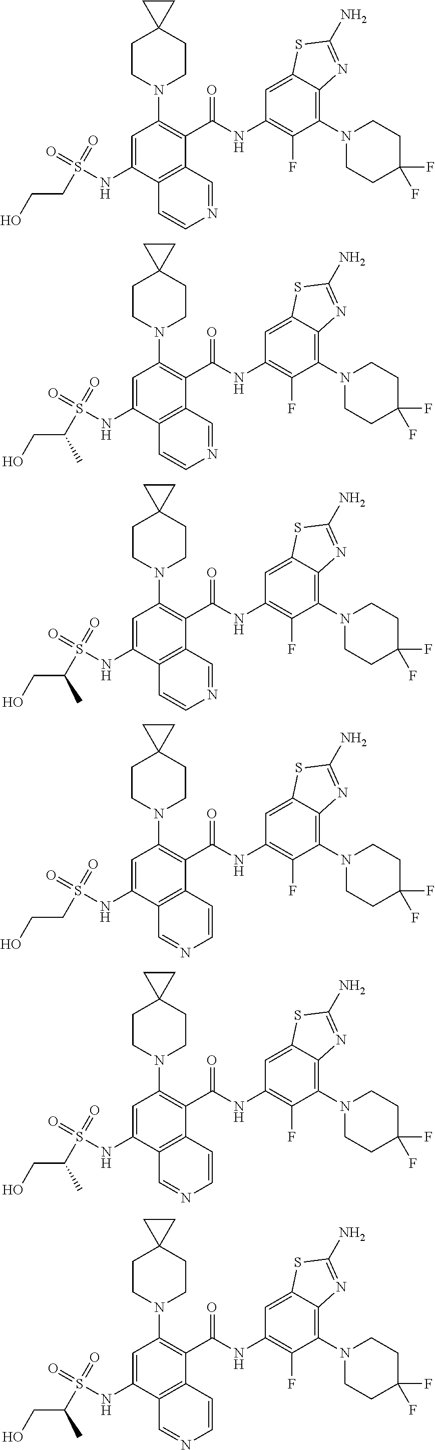 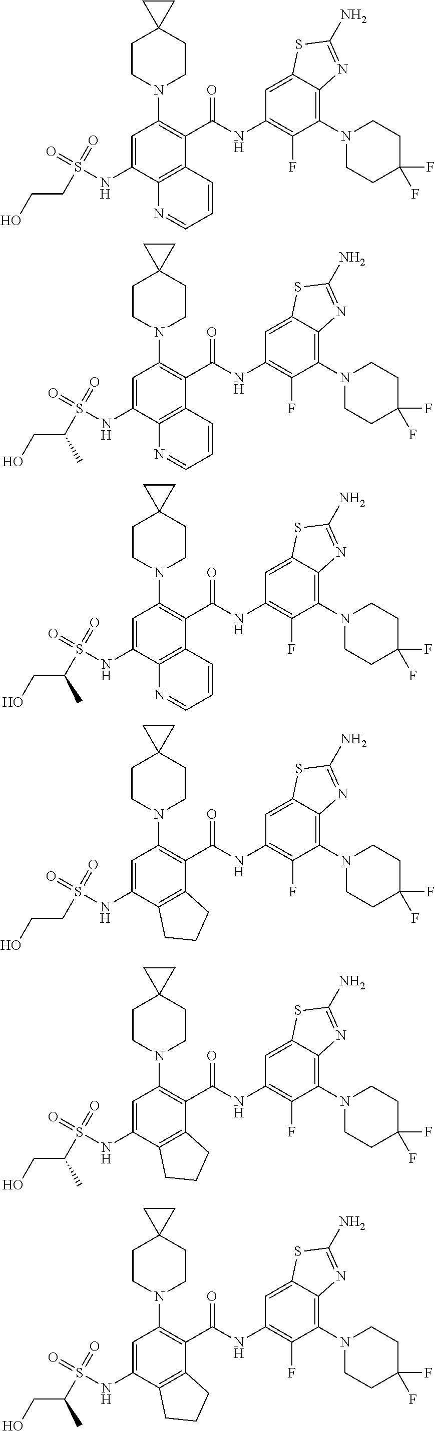 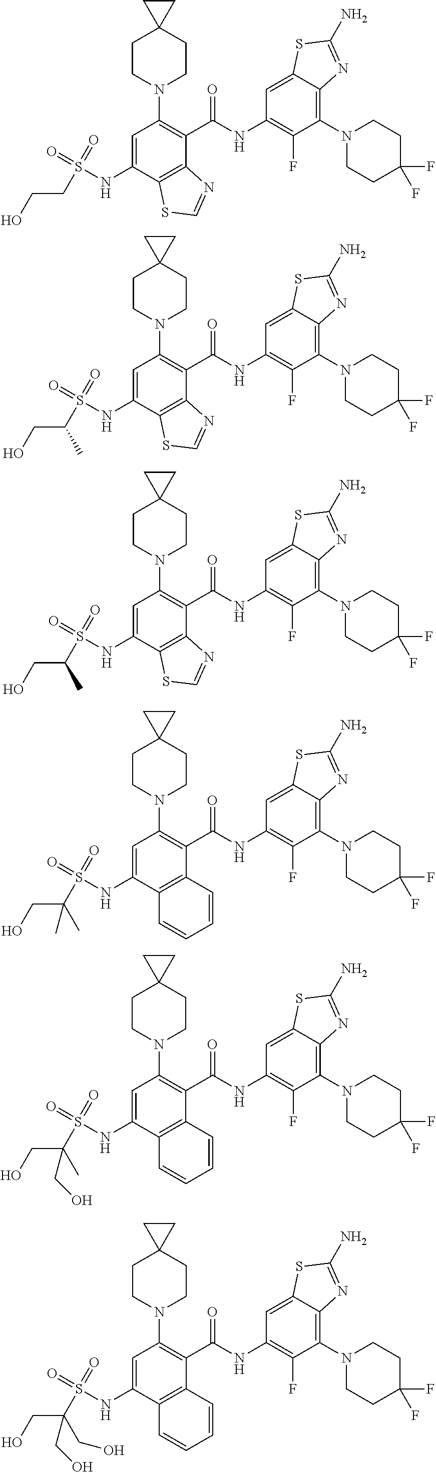 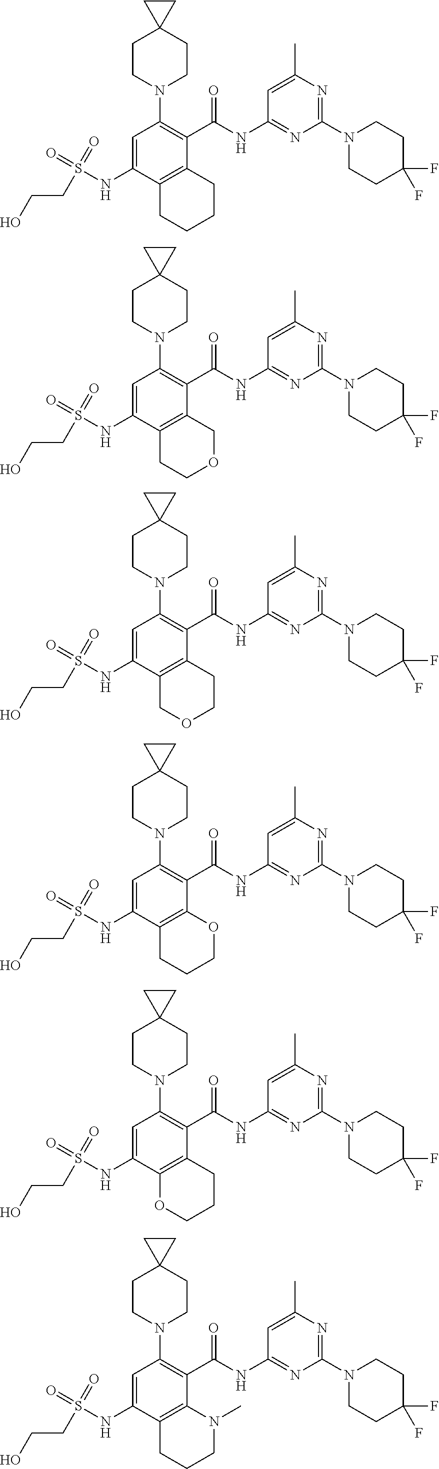 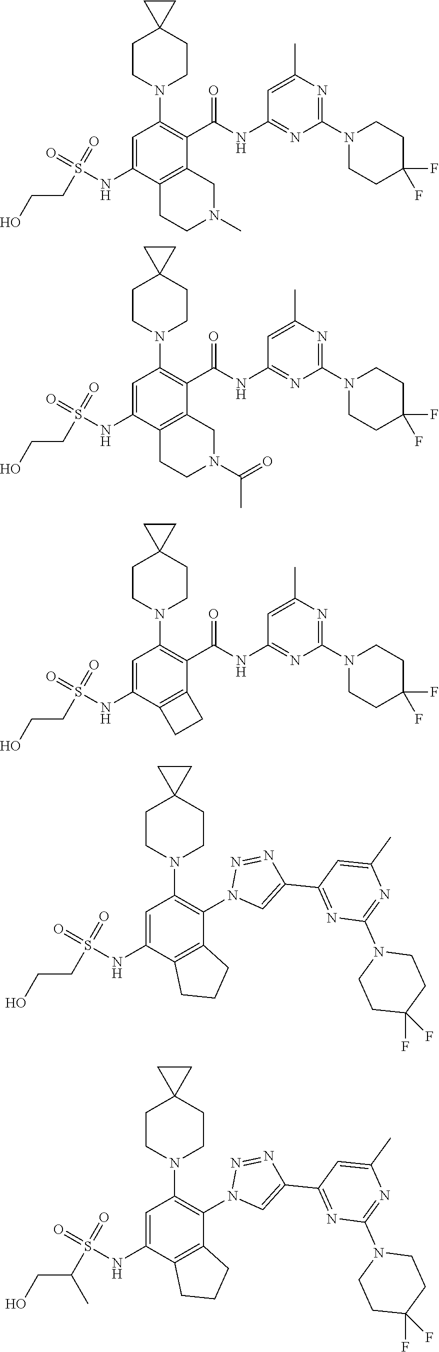 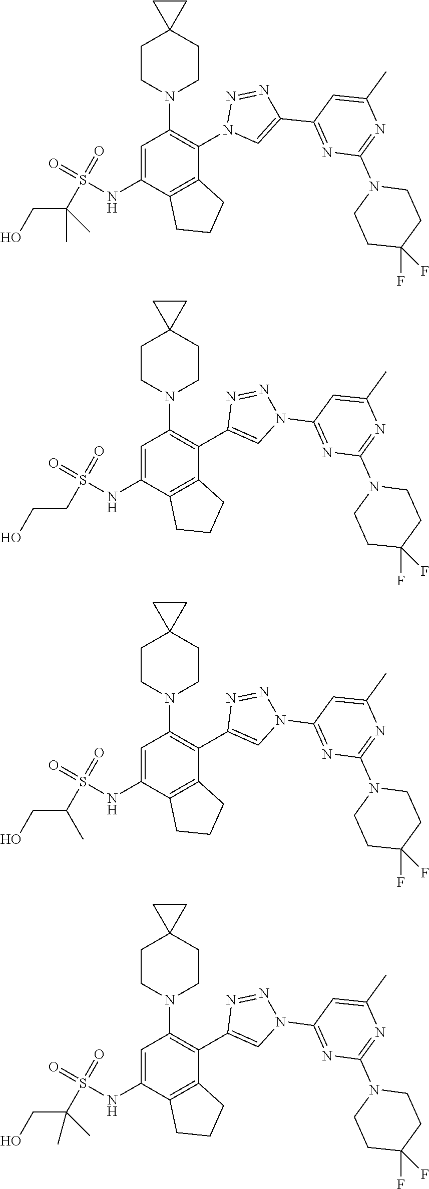 [SMILES: Cc1cc(-c2cn(-c3c(N4CCC5(CC4)CC5)cc(NS(=O)(=O)C(C)(C)CO)c4c3CCC4)nn2)nc(N2CCC(F)(F)CC2)n1 ; Cc1cc(-n2cc(-c3c(N4CCC5(CC4)CC5)cc(NS(=O)(=O)CCO)c4c3CCC4)nn2)nc(N2CCC(F)(F)CC2)n1 ; Cc1cc(-n2cc(-c3c(N4CCC5(CC4)CC5)cc(NS(=O)(=O)C(C)CO)c4c3CCC4)nn2)nc(N2CCC(F)(F)CC2)n1 ; Cc1cc(-n2cc(-c3c(N4CCC5(CC4)CC5)cc(NS(=O)(=O)C(C)(C)CO)c4c3CCC4)nn2)nc(N2CCC(F)(F)CC2)n1] 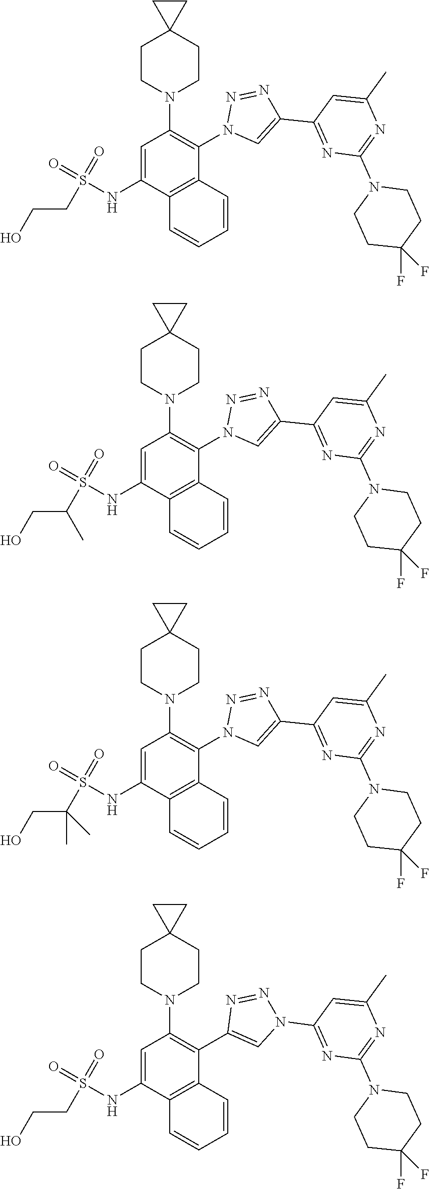 [SMILES: Cc1cc(-c2cn(-c3c(N4CCC5(CC4)CC5)cc(NS(=O)(=O)CCO)c4ccccc34)nn2)nc(N2CCC(F)(F)CC2)n1 ; Cc1cc(-c2cn(-c3c(N4CCC5(CC4)CC5)cc(NS(=O)(=O)C(C)CO)c4ccccc34)nn2)nc(N2CCC(F)(F)CC2)n1 ; Cc1cc(-c2cn(-c3c(N4CCC5(CC4)CC5)cc(NS(=O)(=O)C(C)(C)CO)c4ccccc34)nn2)nc(N2CCC(F)(F)CC2)n1 ; Cc1cc(-n2cc(-c3c(N4CCC5(CC4)CC5)cc(NS(=O)(=O)CCO)c4ccccc34)nn2)nc(N2CCC(F)(F)CC2)n1] 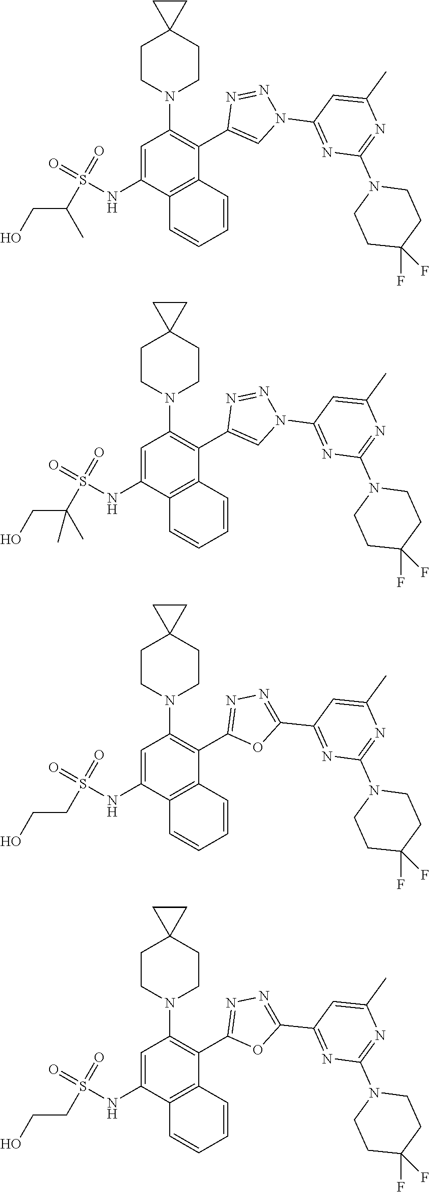 [SMILES: Cc1cc(-n2cc(-c3c(N4CCC5(CC4)CC5)cc(NS(=O)(=O)C(C)CO)c4ccccc34)nn2)nc(N2CCC(F)(F)CC2)n1 ; Cc1cc(-n2cc(-c3c(N4CCC5(CC4)CC5)cc(NS(=O)(=O)C(C)(C)CO)c4ccccc34)nn2)nc(N2CCC(F)(F)CC2)n1 ; Cc1cc(-c2nnc(-c3c(N4CCC5(CC4)CC5)cc(NS(=O)(=O)CCO)c4ccccc34)o2)nc(N2CCC(F)(F)CC2)n1 ; Cc1cc(-c2nnc(-c3c(N4CCC5(CC4)CC5)cc(NS(=O)(=O)CCO)c4ccccc34)o2)nc(N2CCC(F)(F)CC2)n1] 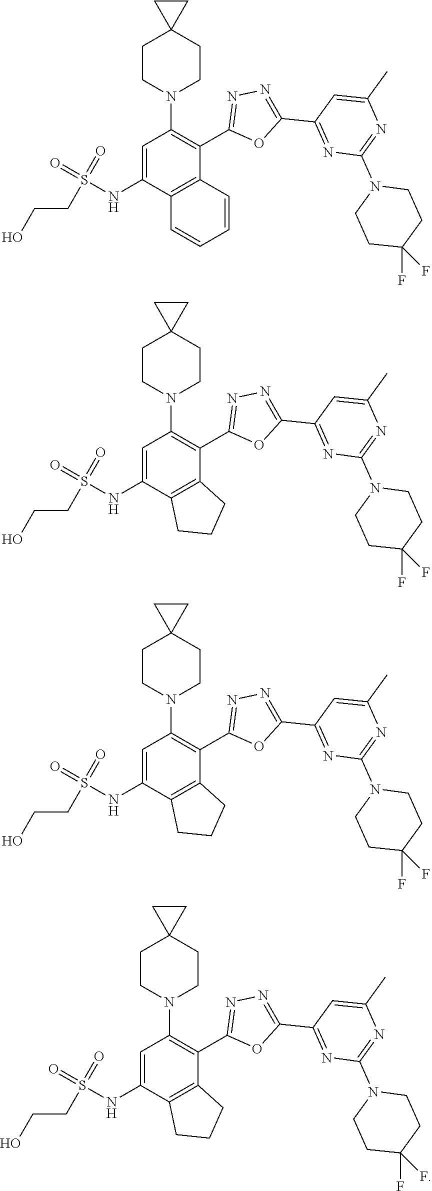 [SMILES: Cc1cc(-c2nnc(-c3c(N4CCC5(CC4)CC5)cc(NS(=O)(=O)CCO)c4ccccc34)o2)nc(N2CCC(F)(F)CC2)n1 ; Cc1cc(-c2nnc(-c3c(N4CCC5(CC4)CC5)cc(NS(=O)(=O)CCO)c4c3CCC4)o2)nc(N2CCC(F)(F)CC2)n1 ; Cc1cc(-c2nnc(-c3c(N4CCC5(CC4)CC5)cc(NS(=O)(=O)CCO)c4c3CCC4)o2)nc(N2CCC(F)(F)CC2)n1 ; Cc1cc(-c2nnc(-c3c(N4CCC5(CC4)CC5)cc(NS(=O)(=O)CCO)c4c3CCC4)o2)nc(N2CCC(F)(F)CC2)n1]
